(19) United States
(12) Patent Application Publication
(10) Pub. No.: US 2025/0215042 A1
(43) Pub. Date: Jul. 3, 2025

(54) MANNOSE-TARGETED COMPOSITIONS

(71) Applicant: GENEVANT SCIENCES GMBH, Basel (CH)

(72) Inventors: Richard J. Holland, Vancouver (CA); James Heyes, Vancouver (CA); Mark Wood, Port Moody (CA); Xin Ye, Richmond (CA); Lorne Ralph Palmer, Vancouver (CA); Eleni Samaridou, Mainz (DE)

(73) Assignee: GENEVANT SCIENCES GMBH, Basel (CH)

(21) Appl. No.: 18/851,013

(22) PCT Filed: Apr. 1, 2023
(86) PCT No.: PCT/IB2023/053285

§ 371 Date: Sep. 25, 2024

Related U.S. Application Data
(60) Provisional application No. 63/326,528, filed on Apr. 1, 2022.

(51) Int. Cl. C07H 21/02; A61K 31/7056; A61K 31/713; C07H 15/26; C12N 5/0784; C12N 5/0786

(52) U.S. Cl. CPC C07H 21/02; A61K 31/7056; A61K 31/713; C07H 15/26; C12N 5/0639; C12N 5/0645

(57) ABSTRACT

The invention provides compounds of formula (I) and salts thereof, wherein R¹, L¹, A, R^{A}, n, L², and R² have any of the values defined in the specification, as well as compositions comprising the compounds and methods for their use.

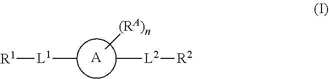

### CROSS-REFERENCE TO RELATED APPLICATION(S)

This patent application claims the benefit of priority of U.S. Provisional Patent Application No. 63/326,528, filed Apr. 1, 2022, which application is herein incorporated by reference.

### BACKGROUND

Macrophages are specialized immune cells involved in the detection, phagocytosis and destruction of pathogens, cancer cells, cellular debris and other foreign substances. In addition, macrophages can also present antigens to T cells and initiate inflammation by releasing cytokines that activate other cells. Macrophages are also infected early on in filovirus (e.g., Ebola or Marburg) infection, leading to increased viral load. They therefore represent a target cell type of interest for a variety of RNAi strategies.

Currently there is a need for compositions and methods that can be used to deliver (e.g., target) therapeutic nucleic acids to macrophages, such as double stranded siRNA, in living subjects.

### BRIEF SUMMARY

The invention provides oligonucleotides, as well as compounds, compositions and methods that can be used to target such oligonucleotides to macrophages.

In one aspect this invention provides a compound of formula I

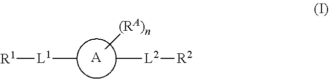

wherein:
R¹ a is targeting ligand that comprises one or more (e.g., 1, 2, 3, 4, 5, or 6) groups of formula:

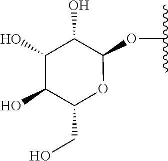 [SMILES: *O[C@H]1O[C@H](CO)[C@@H](O)[C@H](O)[C@@H]1O]

L¹ is absent or a linking group;
L² is absent or a linking group;
R² is an oligonucleotide (e.g., a double stranded siRNA molecule) or a group of formula:

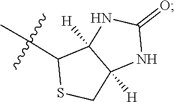 [SMILES: *C1SC[C@@H]2NC(=O)N[C@H]12]

the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydroxy, CN, F, Cl, Br, I, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy; and
n is 0, 1, 2, 3, 4, 5, 6, 7, 8, 9, or 10;
or a salt thereof.

The invention also provides novel oligonucleotides and siRNA conjugates described herein (e.g., those of siRNA conjugates 32d (SEQ ID NOs 9 and 10) and 33c (SEQ ID NOs 15 and 16) from Table 1 below).

The invention also provides a method to deliver an oligonucleotide to an animal (e.g., a mammal, e.g., a human male or human female) comprising administering a compound of formula I or a pharmaceutically acceptable salt thereof to the animal, which may be used in combination with a compound of formula XX or a pharmaceutically acceptable salt thereof.

The invention also provides a method to deliver an oligonucleotide to cells that express mannose receptors CD206 (e.g., macrophages (including Kupffer cell) or dendritic cells) in an animal comprising administering a compound of formula I or a pharmaceutically acceptable salt thereof to the animal, which may be used in combination with a compound of formula XX or a pharmaceutically acceptable salt thereof.

The invention also provides a method to treat a disease selected from the group consisting of acute liver disease, liver inflammation, rheumatoid arthritis, and a flavoviral infection in an animal comprising administering a compound of formula I or a pharmaceutically acceptable salt thereof, to the animal, which may be used in combination with a compound of formula XX or a pharmaceutically acceptable salt thereof.

The invention also provides a method to treat a filovirus infection in an animal comprising administering a therapeutically effective amount of a compound of formula I or a pharmaceutically acceptable salt thereof, to the animal, which may be administered in combination with a compound of formula XX or a pharmaceutically acceptable salt thereof. In certain embodiments, the filovirus is selected from Sudan, Ebola and Marburg virus. In certain embodiments, the virus is Sudan virus. In certain embodiments, the virus is Ebola virus. In certain embodiments, the virus is Marburg virus. The invention also provides a compound of formula I or a pharmaceutically acceptable salt thereof for delivering an oligonucleotide to an animal, which may be used in combination with a compound of formula XX or a pharmaceutically acceptable salt thereof.

The invention also provides a compound of formula I or a pharmaceutically acceptable salt thereof for delivering an oligonucleotide to cells that express mannose receptors CD206 (e.g., macrophages or dendritic cells), which may be used in combination with a compound of formula XX or a pharmaceutically acceptable salt thereof.

The invention also provides a compound of formula I or a pharmaceutically acceptable salt thereof for the prophylactic or therapeutic treatment of a disease selected from the group consisting of acute liver disease, liver inflammation, rheumatoid arthritis, and a flavoviral infection, which may be used in combination with a compound of formula XX or a pharmaceutically acceptable salt thereof.

The invention also provides a compound of formula I or a pharmaceutically acceptable salt thereof for the prophylactic or therapeutic treatment of a filovirus infection, which may be used in combination with a compound of formula XX or a pharmaceutically acceptable salt thereof.

The invention also provides the use of a compound of formula I or a pharmaceutically acceptable salt thereof to prepare a medicament for delivering an oligonucleotide to an animal, which may be used in combination with a compound of formula XX or a pharmaceutically acceptable salt thereof.

The invention also provides the use of a compound of formula I or a pharmaceutically acceptable salt thereof to prepare a medicament for delivering an oligonucleotide to cells that express the mannose receptor CD206, which may be used in combination with a compound of formula XX or a pharmaceutically acceptable salt thereof. CD206 is primarily expressed in macrophages (including Kupffer cell) and dendritic cells but can also be found on a subset of endothelial cells (such as liver sinusoidal endothelial cells) and sperm cells. Accordingly, the compounds and methods described herein can be used to deliver oligonucleotides preferentially to those cell types. Such delivery can be useful, e.g., for treating conditions or diseases such as infectious diseases (sepsis, bacterial, virus, fungi and parasite infection etc); inflammatory diseases (rheumatoid arthritis, atherosclerosis, hepatitis, asthma, osteoarthritis, endometriosis, etc.); tumors and cancers (benign neoplasms, malignant neoplasms, metastasis, etc.); metabolic diseases (obesity, NAFLD, NASH, diabetes, etc.); fibrosis (liver fibrosis, lung fibrosis, renal fibrosis, etc.); and age-related diseases (senescence, neurodegeneration, stroke, Alzheimer's disease, etc.).

The invention also provides the use of a compound of formula I or a pharmaceutically acceptable salt thereof to prepare a medicament for treating a disease selected from the group consisting of acute liver disease, liver inflammation, rheumatoid arthritis, and a flavoviral infection, which may be used in combination with a compound of formula XX or a pharmaceutically acceptable salt thereof.

The invention also provides the use of a compound of formula I or a pharmaceutically acceptable salt thereof to prepare a medicament for treating a filovurus infection in an animal, which may be used in combination with a compound of formula XX or a pharmaceutically acceptable salt thereof.

Combinations described herein comprising a compound of formula I or a pharmaceutically acceptable salt thereof in combination with a compound of formula XX or a pharmaceutically acceptable salt thereof allow for titration of the ratio of the compound of formula I or the pharmaceutically acceptable salt thereof with the compound of formula XX or the pharmaceutically acceptable salt thereof; the ratio of the compound of formula I or the pharmaceutically acceptable salt thereof and the compound of formula XX or the pharmaceutically acceptable salt thereof can be adjusted accordingly for the cell or tissue targeted. The route of administration of the compound of formula I or the pharmaceutically acceptable salt thereof and the compound of formula XX or the pharmaceutically acceptable salt thereof may be the same or different.

In certain embodiments, the combination is in the form of a kit that comprises,
a) a compound of formula I or a pharmaceutically acceptable salt thereof;
b) a compound of formula XX or a pharmaceutically acceptable salt thereof;
c) optionally, packaging material containing the compound of formula I or a pharmaceutically acceptable salt thereof and the compound of formula XX or a pharmaceutically acceptable salt thereof; and
d) optionally instructions for use, e.g., for administering or using the compound of formula I in combination with the compound of formula XX.

Certain embodiments also provide methods, uses and compositions comprising the combination of a compound of formula I with a compound of the following formula XX:

T⁵-L-[PEGMAₘ-M²ₙ]ᵥ-[DMAEMA_{q}-PAAᵣ-BMAₛ]_{w} (XX)

wherein:
PEGMA is polyethyleneglycol methacrylate residue with 2-20 ethylene glycol units;
M² is a methacrylate residue selected from the group consisting of
   a (C₄-C₁₈)alkyl-methacrylate residue;
   a (C₄-C₁₈)branched alkyl-methacrylate residue;
   a cholesteryl methacrylate residue;
   a (C₄-C₁₈)alkyl-methacrylate residue substituted with one or more fluorine atoms; and
   a (C₄-C₁₈)branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa;
w is 1 to 25 kDa;
T⁵ is a targeting moiety; and
L is absent or is a linking moiety.

In certain embodiments, the compound of formula I and compound of formula XX are administered separately

In certain embodiments, the compound of formula XX is administered after administration of the compound of formula I.

In certain embodiments, the compound of formula I and compound of formula XX are administered together within a single composition.

In certain embodiments, the compound of formula XX is Mannose ERP 40

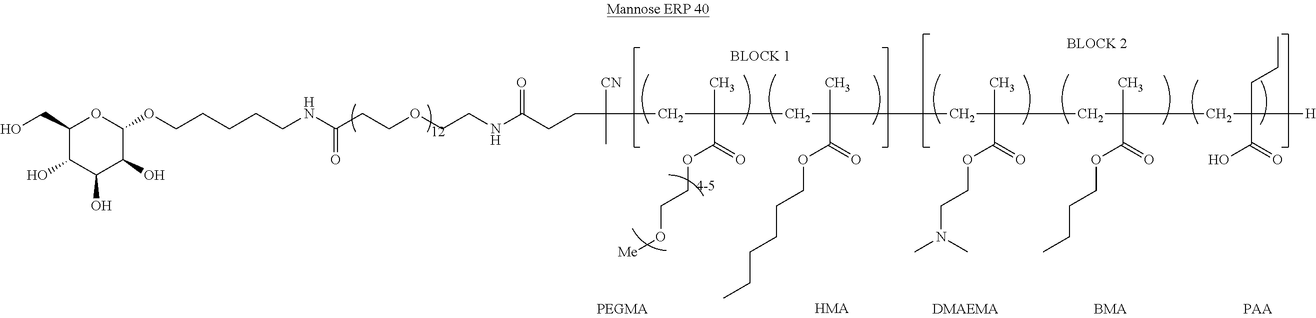

The invention also provides synthetic intermediates and methods disclosed herein that are useful to prepare compounds of formula I.

Other objects, features, and advantages of the present invention will be apparent to one of skill in the art from the following detailed description and figures.

### BRIEF DESCRIPTION OF THE FIGURES

FIG. 1: Illustrates Expression of mannose receptor CD206 in M1 and M2 macrophages derived from human CD14+ monocytes (Example 11).

FIG. 2: Illustrates binding of mono-, di- and tetra-valent mannose ligand complexes to M1 and M2 macrophages (Example 12).

FIG. 3. Illustrates binding of tetra-, hexa- and Octa-valent mannose ligand complexes to M1 macrophages (Example 12).

FIG. 4. Illustrates uptake of fluorescent labelled mannose-siRNA conjugate by M1 macrophage in vitro (Example 13).

FIG. 5. Illustrates that D-Mannose competes with mannose-siRNA conjugate in M1 macrophage binding (Example 14).

FIG. 6. Illustrates uptake of fluorescent labelled mannose-siRNA conjugate by macrophages and dendritic cells in vitro (Example 15).

FIG. 7. Illustrates hexavalent mannose conjugated siRNA enabled gene silencing in M1 macrophage in vitro when treated together with mannose-ERP (Example 16).

FIG. 8. Illustrates delivery of hexa-mannose-siCD45 to thioglycolate elicited periMacs in vivo (Example 17).

FIG. 9. Illustrates target gene silencing with hexa-mannose-siCD45 and mannose-ERP in CD206+ periMacs in thioglycolate mouse model (Example 17).

FIG. 10. Illustrates combination treatment of hexa-mannose-siRNA and GalNAc-siRNA demonstrated anti-viral potency in a guinea pig viral challenge model (Example 18).

FIG. 11. Illustrates hexavalent mannose conjugated siRNA and mannose-ERP enabled gene silencing in mouse liver and spleen macrophages in vivo (Example 19).

FIG. 12. Illustrates dose response of mannose-ERP in liver macrophages in vivo (Example 20).

FIG. 13. Illustrates dose Response of hexa-mannose-siRNA in liver and spleen macrophages in vivo (Example 21).

FIG. 14. Illustrates duration of Effect of hexa-mannose-siRNA with mannose-ERP in liver and spleen macrophages in vivo (Example 22).

FIG. 15. Illustrates multi-dosing of mannose conjugate with ERP improves target silencing in vivo (Example 23).

In the application, including Figures, Examples and Schemes, it is to be understood that an oligonucleotide, in one embodiment, can be a double stranded siRNA molecule.

### DETAILED DESCRIPTION

GalNAc-conjugated short interfering RNA (siRNA) are a modality for mediating RNA interference (RNAi) in hepatocytes. They comprise two important components; a GalNAc targeting ligand, which binds to the asialoglycoprotein receptor (ASGPr) found on the surface of hepatocytes to mediate uptake, and the siRNA oligonucleotide, that once delivered to the cytoplasm of hepatocytes can mediate destruction of specific mRNA sequences (determined by the sequence of the siRNA) to reduce expression of the associated protein product.

While siRNA delivery to hepatocytes has been demonstrated with this class of therapeutics, successful application to other cell types has been more problematic. Suitable receptors must be identified, which are expressed relatively selectively on the target cell surface and in large numbers. Then, appropriate ligands must be identified that bind with high specificity and affinity to the target receptor. A significant third hurdle is endosomal escape; once bound to a receptor, the ligand conjugate is now taken up by the cell and entrapped in an endosome, from which it must escape to reach the cytoplasm. Despite lacking an active endosomal escape mechanism, GalNAc conjugates appear to mediate activity regardless, possibly because the receptor is so abundantly expressed and quickly recycled (˜15 minutes) following uptake. The sheer number of conjugate molecules taken up by hepatocytes may obviate the need for an active endosomal escape. This has not been true for other ligand-based siRNA conjugate systems.

As described herein, a suitable target receptor for this approach on macrophages, the mannose receptor (CD206), has been identified. Novel ligands have been designed, synthesized and tested showing binding and uptake. Under most circumstances, co-administration of an endosomal release polymer (ERP) is required for gene silencing, both in vitro and in vivo.

As used herein, the following terms have the meanings ascribed to them unless specified otherwise.

The term “conjugate” as used herein includes compounds of formula (I) that comprise an oligonucleotide (e.g., an siRNA molecule) linked to a targeting ligand. Thus, the terms compound and conjugate may be used herein interchangeably.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-4 (e.g., 1, 2, 3, or 4) modified and/or unmodified deoxythymidine (t or dT) nucleotides, 1-4 (e.g., 1, 2, 3, or 4) modified (e.g., 2′OMe) and/or unmodified uridine (U) ribonucleotides, and/or 1-4 (e.g., 1, 2, 3, or 4) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA of the invention to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP) or 0-benzotriazol-1-yl-N,N,N′,N′-tetramethyluronium hexafluorophosphate (HBTU).

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “oligonucleotide” includes nucleotides containing up to about 60 nucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), small activating RNA (saRNA), asymmetrical interfering RNA (aiRNA), microRNA (miRNA), tRNA, rRNA, tRNA, viral RNA (vRNA). Accordingly, the term “oligonucleotide” refers to a polymer or oligomer of nucleotide or nucleoside monomers consisting of naturally-occurring bases, sugars and intersugar (backbone) linkages. The term “oligonucleotide” also includes polymers or oligomers comprising non-naturally occurring monomers, or portions thereof. Such modified or substituted oligonucleotides may have properties, for example, of enhanced cellular uptake, reduced immunogenicity and/or increased stability in the presence of nucleases.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

“Filovirus,” as used herein, refers to members of the filovridae family, and includes but is not limited to the genera Ebolavirus, and Marburgvirus, Cuevavirus, Dianlovirus, Striavirus and Thamnovirus. Ebolavirus includes without limitation Bombali ebolavirus, Bundibugyo ebolavirus, Reston ebolavirus, Sudan ebolavirus, Tai Forest ebolavirus, Zaire ebolavirus.

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ringsystem having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring system, such as, for example, bicyclo[2.2.1]heptane, pinane, bicyclo[2.2.2]octane, adamantane, norborene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having the overall having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

The term “animal” includes mammalian species, such as a human, mouse, rat, dog, cat, hamster, guinea pig, rabbit, livestock, and the like.

The term “alkylamino” includes a group of formula —N(H)R, wherein R is an alkyl as defined herein.

The term “dialkylamino” includes a group of formula —NR₂, wherein each R is independently an alkyl as defined herein.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. Unless otherwise specifically noted, when a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Unless stated otherwise herein, the term “about”, when used in connection with a value or range of values, means plus or minus 5% of the stated value or range of values.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, CA). However, a larger or smaller scale of synthesis is also within the scope of this invention. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Embodiments of the Invention

One aspect of the invention is a compound of formula I, as set forth about in the Summary of the Invention, or a salt thereof.

In one embodiment of the compound of formula I, R¹ a is targeting ligand;
L¹ is absent or a linking group;
L² is absent or a linking group;
R² is a double stranded siRNA;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, and C₁₋₈ alkyl that is optionally substituted with one or more groups independently selected from halo, hydroxy, and C₁₋₃ alkoxy; and
n is 0, 1, 2, 3, 4, 5, 6, 7, 8, 9, or 10.

In one embodiment R¹ is:

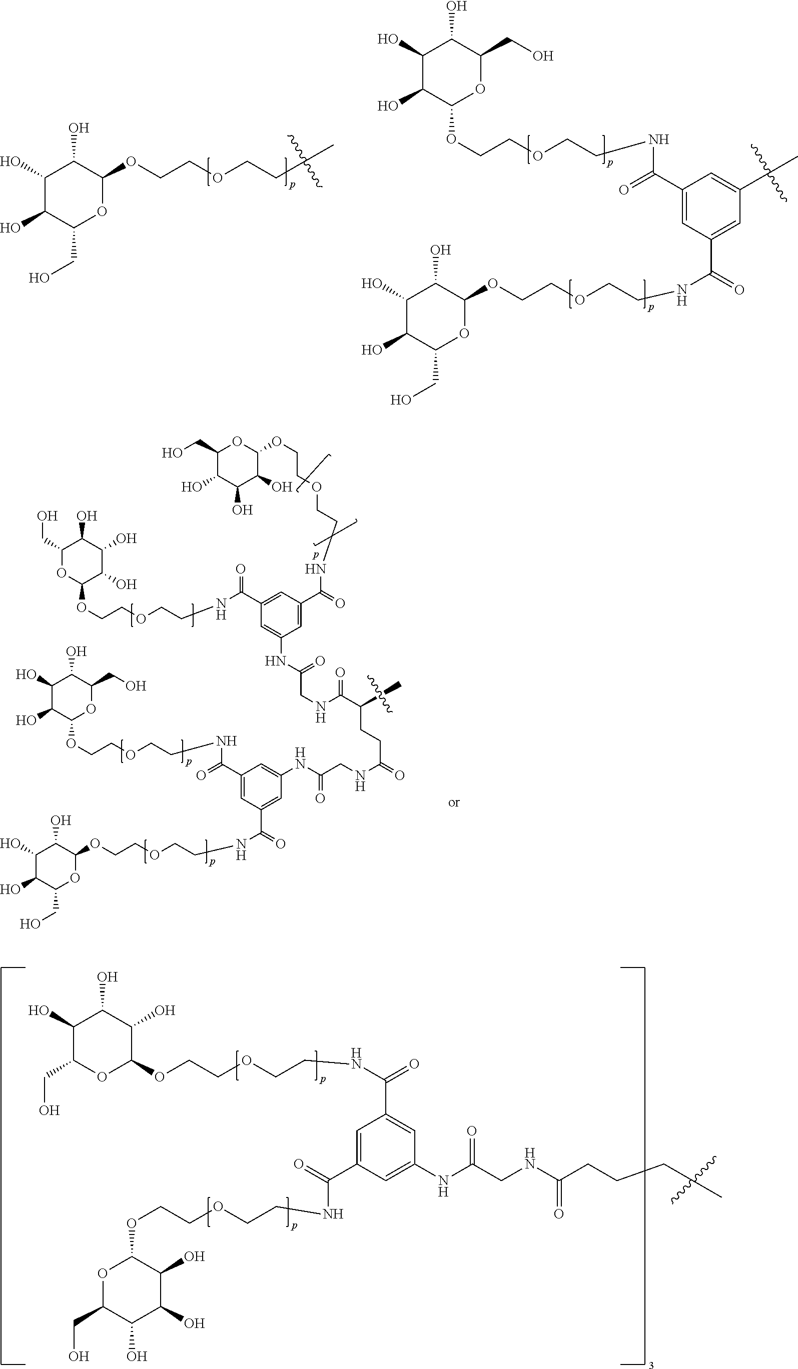

or a salt thereof, wherein each p is independently selected from the group consisting of 0, 1, 2, 3, 4, or 5.

In one embodiment R¹ is:

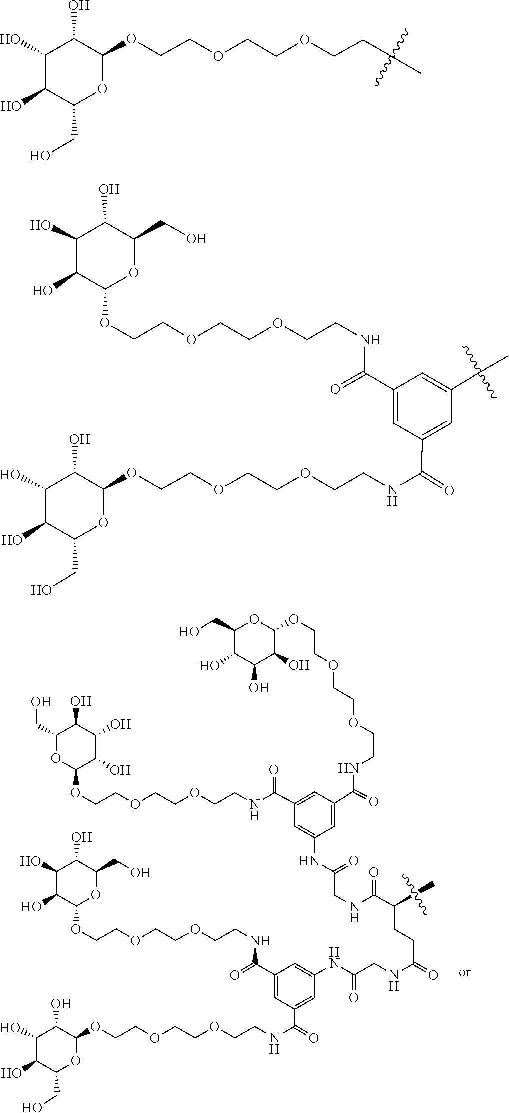

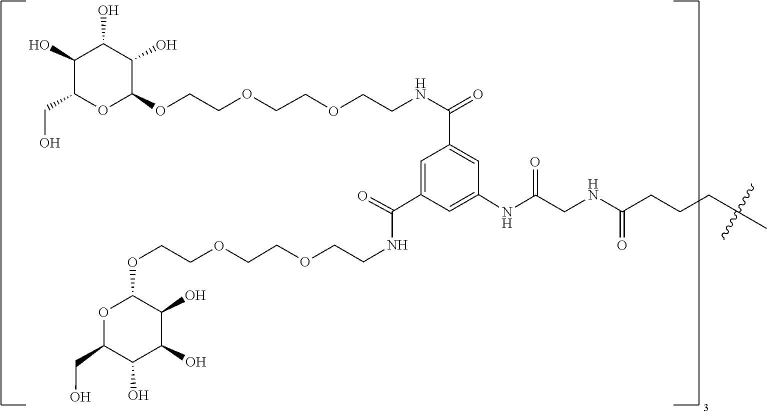

or a salt thereof.

In one embodiment linking groups L¹ is a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In one embodiment L² is connected to R² through —O—.

In one embodiment L¹ is selected from the group consisting of:

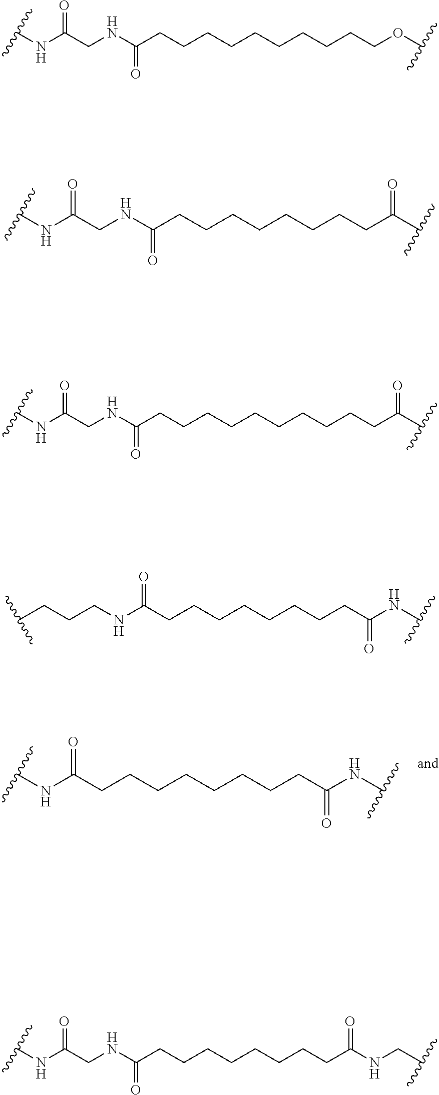 [SMILES: *NC(=O)CNC(=O)CCCCCCCCCCO* ; *NC(=O)CNC(=O)CCCCCCCCC(*)=O ; *NC(=O)CNC(=O)CCCCCCCCCCC(*)=O ; *CCCNC(=O)CCCCCCCCC(=O)N* ; *NC(=O)CCCCCCCCC(=O)N* ; *CNC(=O)CCCCCCCCC(=O)NCC(=O)N*]

and salts thereof.

In one embodiment L¹ is selected from the group consisting of:

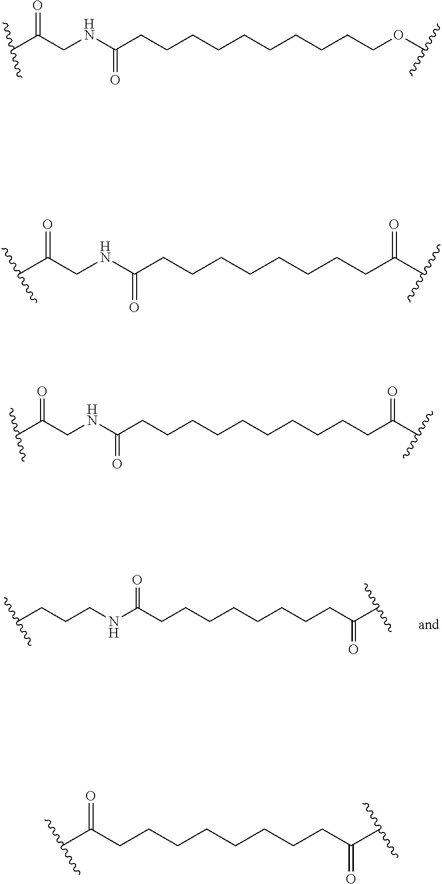 [SMILES: *OCCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O]

and salts thereof.

In one embodiment L¹ is selected from the group consisting of:

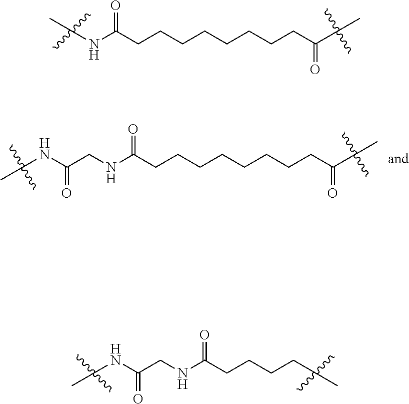 [SMILES: *NC(=O)CCCCCCCCC(*)=O ; *NC(=O)CNC(=O)CCCCCCCCC(*)=O ; *CCCCC(=O)NCC(=O)N*]

In one embodiment L² is —CH₂—O— or —CH₂—CH₂—O—.

In one embodiment a compound of formula I has the following formula (Ic):

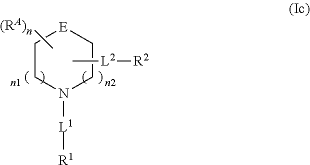

wherein E is —O— or —CH₂—;
n is selected from the group consisting of 0, 1, 2, 3, and 4; and
n1 and n2 are each independently selected from the group consisting of 0, 1, 2, and 3;
or a salt thereof.

In certain embodiments a compound of formula (Ic) is selected from the group consisting of:

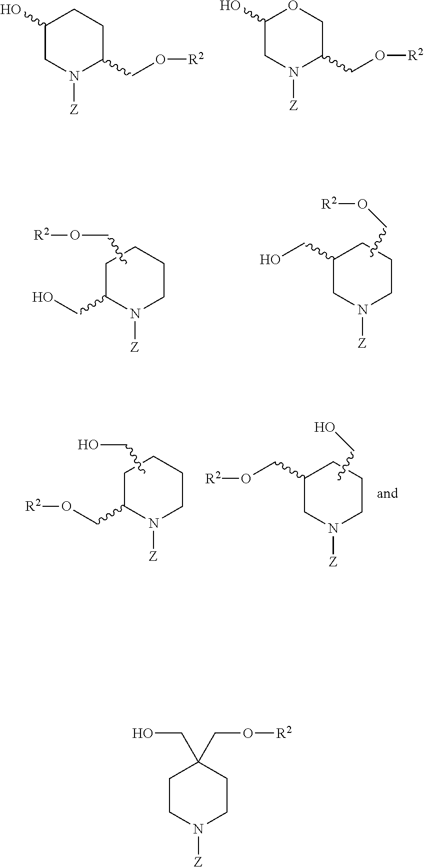

wherein Z is -L¹-R¹;
and salts thereof.

In one embodiment the -A-L²-R² moiety is:

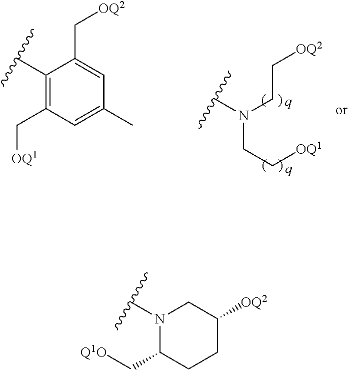

wherein:
Q¹ is hydrogen and Q² is R²; or Q¹ is R² and Q² is hydrogen; and
each q is independently 0, 1, 2, 3, 4 or 5;
or a salt thereof.

In one embodiment a compound of formula (I) is selected from the group consisting of:

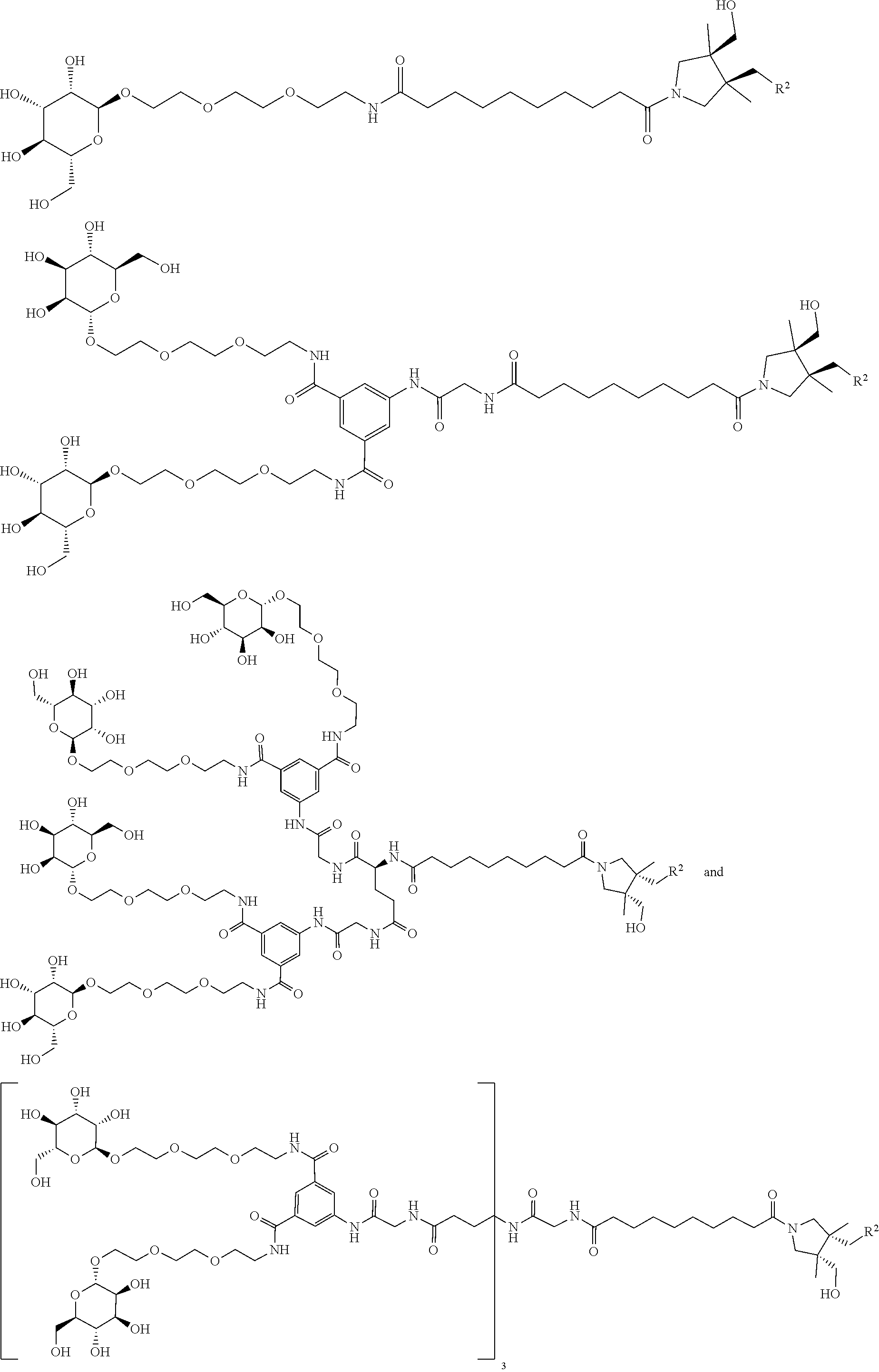

and salts thereof.

In one embodiment R¹ is selected from the group consisting of:

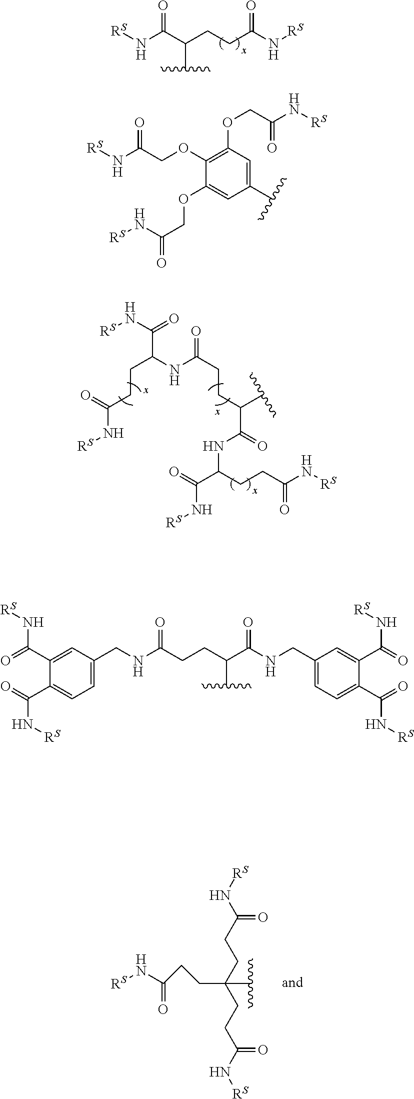

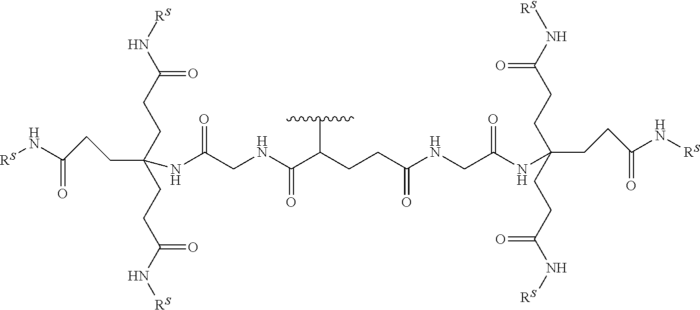

wherein R^{S} is

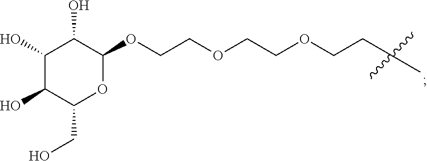 [SMILES: *CCOCCOCCO[C@H]1O[C@H](CO)[C@@H](O)[C@H](O)[C@@H]1O]

n is 2, 3, or 4;
x is 1 or 2.

In one embodiment L¹ is selected from the group consisting of:

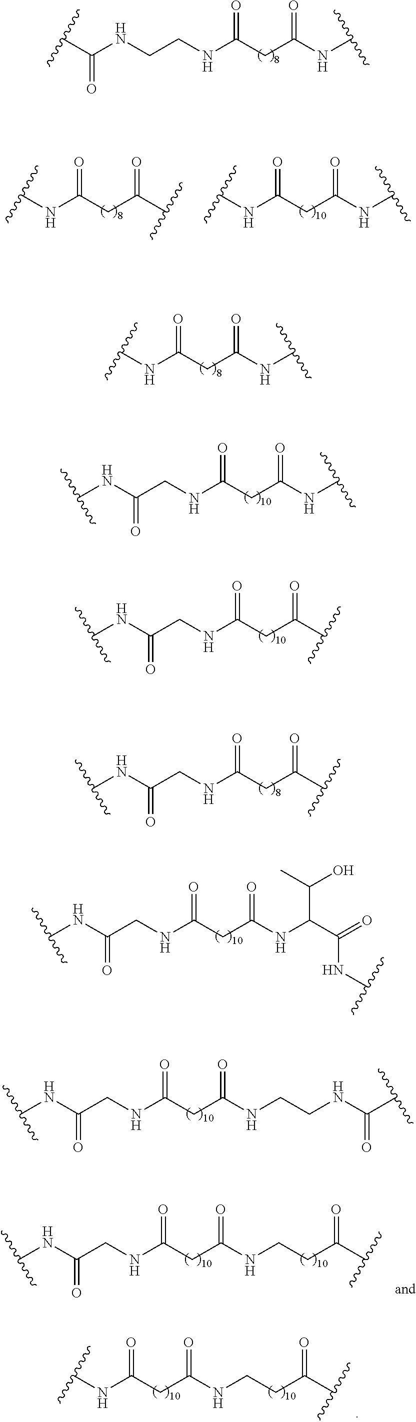

In one embodiment L¹ is selected from the group consisting of:

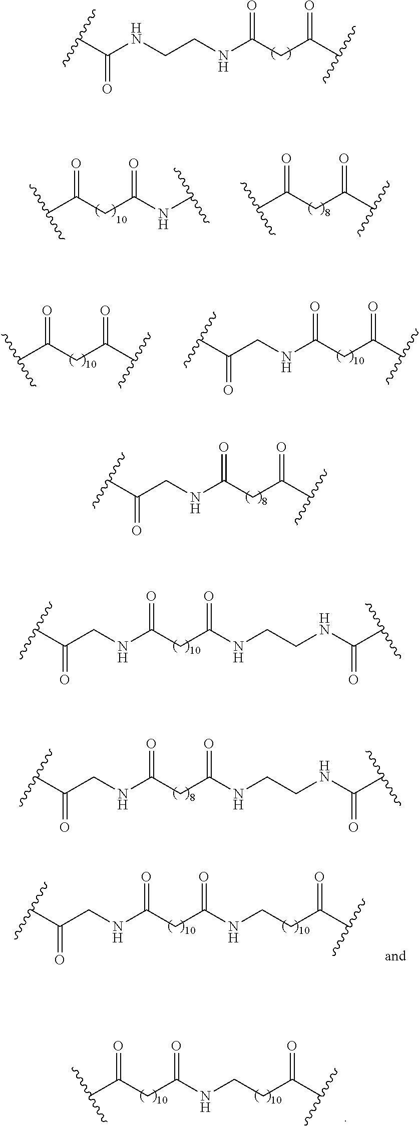

In one embodiment A is absent, phenyl, pyrrolidinyl, or cyclopentyl.

In one embodiment L² is C₁₋₄ alkylene-O— that is optionally substituted with hydroxy.

In one embodiment L² is —CH₂O—, —CH₂CH₂O—, or —CH(OH)CH₂O—.

In one embodiment each R^{A} is independently hydroxy or C₁₋₈ alkyl that is optionally substituted with hydroxyl.

In one embodiment each R^{A} is independently selected from the group consisting of hydroxy, methyl and —CH₂OH.

In one embodiment a compound of formula I has the following formula (Ig):

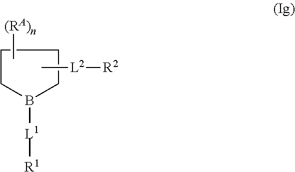

wherein B is —N— or —CH—;
L¹ is absent or is a linking group;
L² is C₁₋₄ alkylene-O— that is optionally substituted with hydroxyl or halo;
n is 0, 1, or 2;
or a salt thereof.

In one embodiment a compound of formula I has the following formula (Ig):

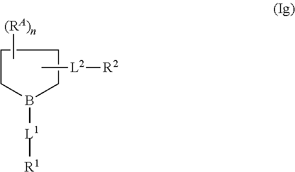

wherein B is —N— or —CH—;
L¹ is absent or is a linking group;
L² is C₁₋₄ alkylene-O— that is optionally substituted with hydroxyl or halo;
n is 0, 1, 2, 3, 4, 5, 6, or 7;
or a salt thereof.

In one embodiment a compound of formula I has the following formula (Ig):

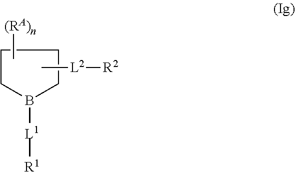

wherein B is —N— or —CH—;
L¹ is absent or is a linking group;
L² is C₁₋₄ alkylene-O— that is optionally substituted with hydroxyl or halo;
n is 0, 1, 2, 3, or 4;
or a salt thereof.

In one embodiment the compound of formula I is:

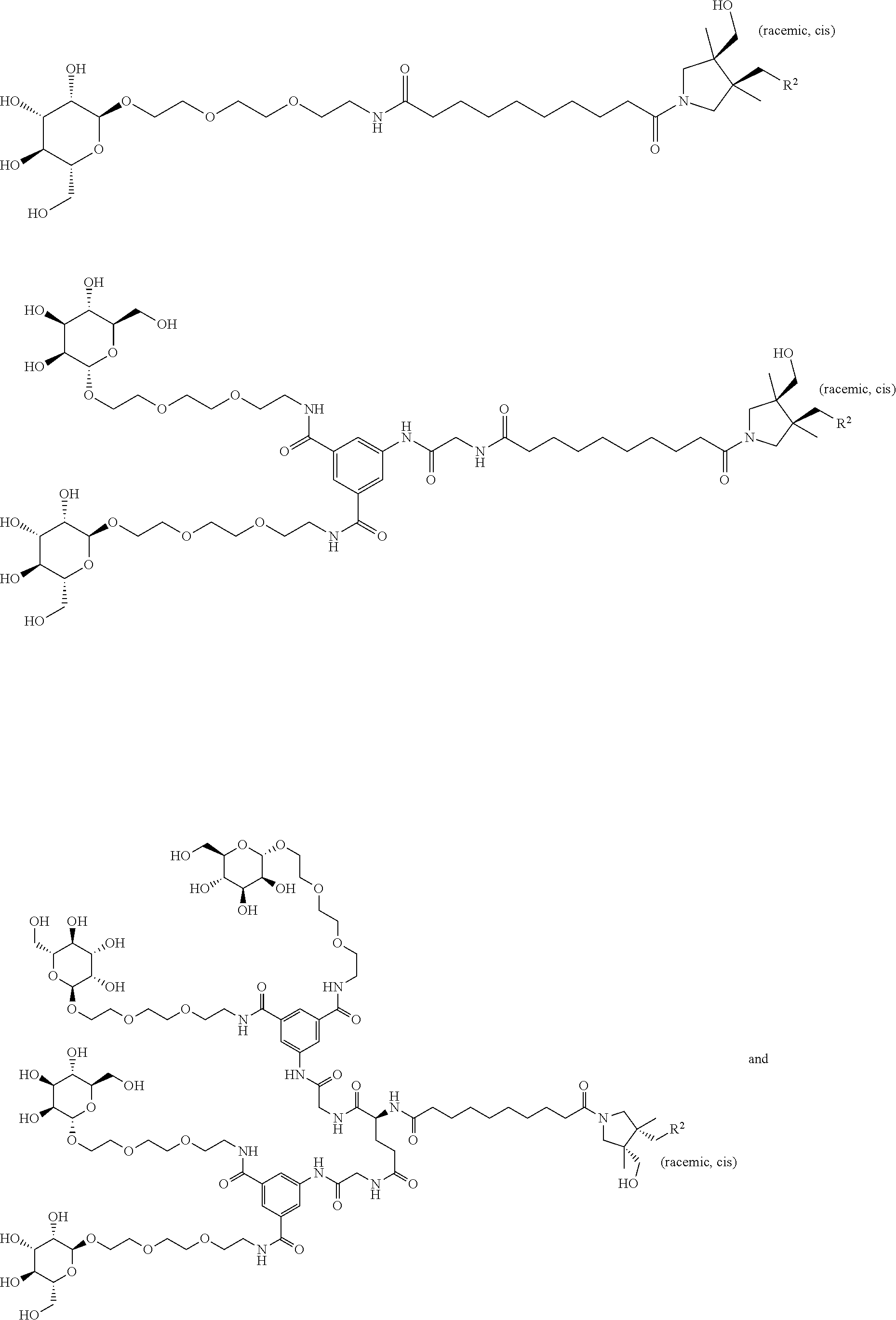

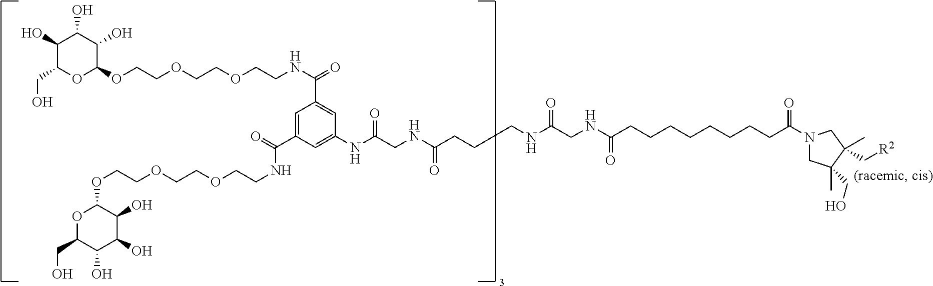

and salts thereof.

In one embodiment, R² is a biotin residue of the following formula:

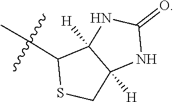 [SMILES: *C1SC[C@@H]2NC(=O)N[C@H]12]

Streptavidin is a 52 kDa protein (tetramer) originally isolated from the bacterium Streptomyces avidinii. Streptavidin is also available from recombinant sources. Streptavidin homo-tetramers have an extraordinarily high affinity for biotin, with a dissociation constant (K_{d}) on the order of 10⁻¹⁴ mol/L (Green NM, 1975, Advances in Protein Chemistry. 29: 85-133). Accordingly, compounds of formula (I) wherein R² is a biotin residue can associate with streptavidin (which may be further functionalised with a fluorescent dye) to provide reagents that are useful for carrying out biological assays including competitive inhibition assays.

One aspect of this invention is pharmaceutical composition comprising a compound of formula I or a pharmaceutically acceptable salt thereof, and a pharmaceutically acceptable carrier, which may be in combination with a compound of formula XX or a pharmaceutically acceptable salt thereof.

Another aspect of this invention is a method to deliver a double stranded siRNA to the liver of an animal comprising administering a compound of formula I or a pharmaceutically acceptable salt thereof, to the animal, which may be in combination with a compound of formula XX or a pharmaceutically acceptable salt thereof.

Certain embodiments of the invention provide a compound of formula (I) or a pharmaceutically acceptable salt thereof for use in medical therapy, which may be in combination with a compound of formula XX or a pharmaceutically acceptable salt thereof.

In certain embodiments, the animal is a mammal, such as a human (e.g., an HBV infected patient).

In one embodiment, the nucleic acid molecule (e.g., siRNA) is attached to the reminder of the compound through the oxygen of a phosphate at the 3′-end of the sense strand.

In one embodiment the compound or salt is administered subcutaneously.

In one embodiment, the compounds and methods described herein can be used to deliver oligonucleotides preferentially to those cell types. Such delivery can be useful, e.g., for treating conditions or diseases such as infectious diseases (sepsis, bacterial, virus, fungi and parasite infection etc); inflammatory diseases (rheumatoid arthritis, atherosclerosis, hepatitis, asthma, osteoarthritis, endometriosis, etc.); tumors and cancers (benign neoplasms, malignant neoplasms, metastasis, etc.); metabolic diseases (obesity, NAFLD, NASH, diabetes, etc.); fibrosis (liver fibrosis, lung fibrosis, renal fibrosis, etc.); and age-related diseases (senescence, neurodegeneration, stroke, Alzheimer's disease, etc.).

Another aspect of this invention is a method to deliver an oligonucleotide to a cell, comprising contacting the cell with a compound of formula I or a pharmaceutically acceptable salt thereof, wherein the cell is in vitro or in vivo.

In one embodiment, the method or use comprises delivery to macrophages in the liver

Another aspect of this invention is a combination that comprises:
a) a compound of formula I or a pharmaceutically acceptable salt thereof; and
b) a compound of formula XX or a pharmaceutically acceptable salt thereof.

Another aspect of this invention is a kit that comprises,
a) a compound of formula I or a pharmaceutically acceptable salt thereof;
b) a compound of formula XX or a pharmaceutically acceptable salt thereof;
c) optionally, packaging material containing the compound of formula I or the pharmaceutically acceptable salt thereof and the compound of formula XX or the pharmaceutically acceptable salt thereof, and
d) optionally instructions for use, e.g., for administering or using the compound of formula I or the pharmaceutically acceptable salt thereof in combination with the compound of formula XX or the pharmaceutically acceptable salt thereof.

When a compound comprises a group of the following formula:

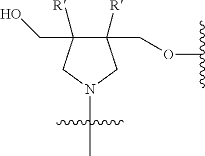

there are four stereoisomers possible on the ring, two cis and two trans. Unless otherwise noted, the compounds of the invention include all four stereoisomers about such a ring. In one embodiment, the two R′ groups are in a cis conformation. In one embodiment, the two R′ groups are in a trans conformation.

### EXAMPLES

The present invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results.

### Example 1. Preparation of Compound 9

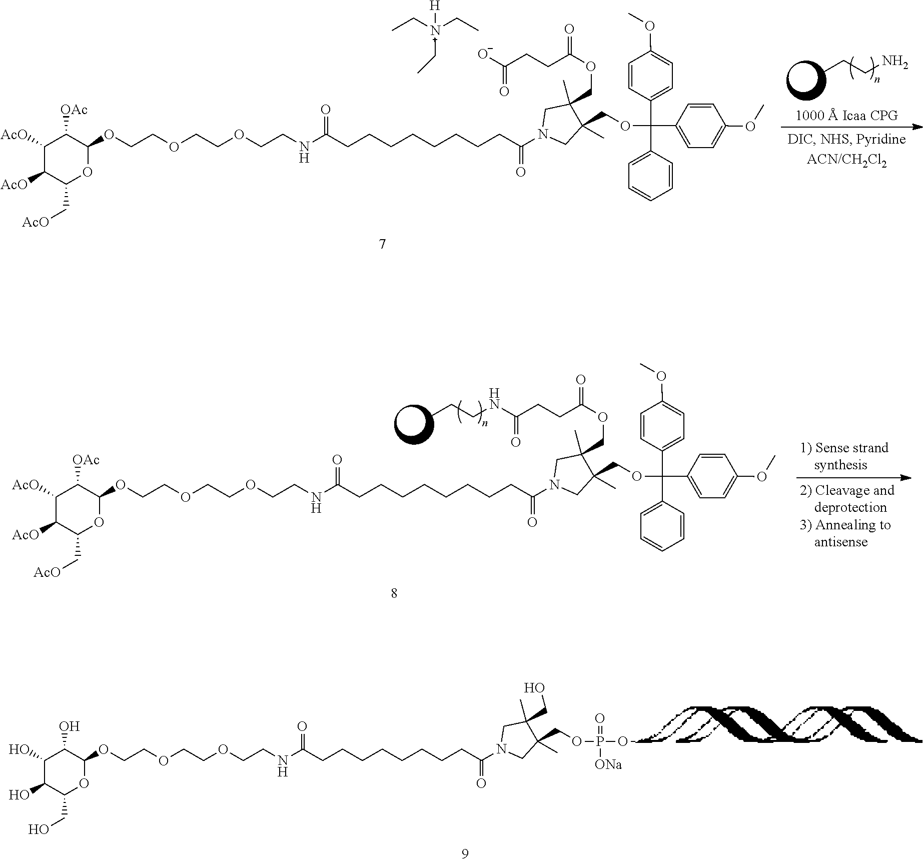

To a solution of Compound 7 (2.0 g, 1.7 mmol), N-hydroxysuccinimide (38.1 mg, 0.3 mmol) and pyridine (0.4 mL, 5.0 mmol) in 1:1 MeCN/DCM (50 mL) was added 1000 Å 1caa CPG (long chain aminoalkyl, controlled pore glass, 15 g). The suspension was gently agitated at room temperature for 16 hours. Upon completion, the CPG was filtered, rinsed with DCM, air dried, then suspended in a solution of 10% acetic anhydride, 5% N-methylimidazole and 5% pyridine in THE (75 mL). After 2 hours the capping solution was filtered and the CPG rinsed with THF (50 mL), MeCN (50 mL) and DCM (50 mL) then dried under high vacuum for 16 hours. The succinate 7 loading efficiency was 38 μmol/g (determined by standard UV/Vis DMT assay @504 nm). The resulting mono-valent mannose CPG solid support was employed in automated oligonucleotide synthesis using standard procedures. Nucleotide cleavage and deprotection, with concurrent mannose acetate deprotection, afforded the 3′ conjugated, mono-mannose sense strand. After purification by dual HPLC, the corresponding antisense strand was annealed to form a siRNA duplex 9.

The intermediate Compound 7 was prepared from Compound 1 as described below.

a. Synthesis of Compound 2

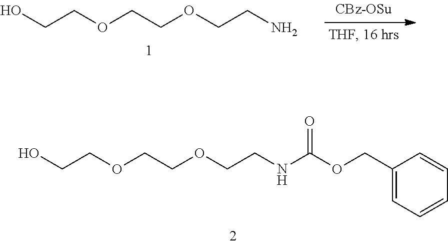 [SMILES: NCCOCCOCCO ; O=C(NCCOCCOCCO)OCc1ccccc1]

A solution of 2-[2-(2-aminoethoxy)ethoxy]ethanol 1 (20.0 g, 134.1 mmol) and benzyl 2,5-dioxopyrrolidin-1-yl carbonate (33.4 g, 134.1 mmol) in THF (400 mL) was stirred for 16 hours at room temperature. Upon completion, the solution was concentrated to dryness, redissolved in EtOAc (400 mL), washed with 0.25M NaOH (250 mL) and brine (1×200 mL), dried (MgSO₄), filtered, and concentrated in-vacuo. Purification by automated column chromatography (0-7% MeOH in CH₂Cl₂) gave benzyl N-{2-[2-(2-hydroxyethoxy)-ethoxy]ethyl}carbamate 2 (28.5 g, 80%).

b. Synthesis of Compound 3

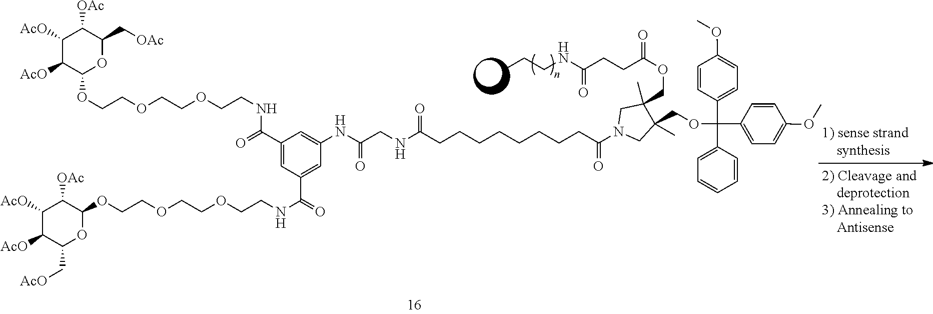

To a cooled (0° C.) solution of 1,2,3,4,6-Penta-O-acetyl-alpha-D-mannopyranose (64.3 g, 164.9 mmol) and benzyl N-{2-[2-(2-hydroxyethoxy)ethoxy]ethyl}carbamate 2 (46.7 g, 164.8 mmol) in anhydrous DCM (400 mL) was added dropwise boron trifluoride diethyl etherate (163 mL, 1.32 mol) over 1 hour. The reaction was warmed to room temperature and stirred for 72 hours. Upon completion, the reaction was carefully poured into ice water (1 L). The organic layer was separated, washed with saturated NaHCO₃ (200 mL) and brine (200 mL), dried (MgSO₄), filtered, and concentrated in vacuo. Purification by automated column chromatography (5% MeOH/DCM) gave compound 3 (46.6 g, 46%).

c. Synthesis of Compound 4

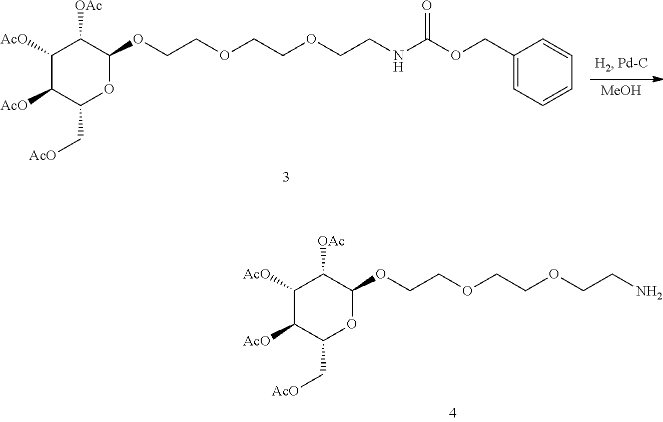 [SMILES: "MeOH" ; CC(=O)OC[C@H]1O[C@H](OCCOCCOCCNC(=O)OCc2ccccc2)[C@@H](OC(C)=O)[C@@H](OC(C)=O)[C@@H]1OC(C)=O ; CC(=O)OC[C@H]1O[C@H](OCCOCCOCCN)[C@@H](OC(C)=O)[C@@H](OC(C)=O)[C@@H]1OC(C)=O]

To a solution of [(2R,3R,4S,5S)-3,4,5-tris(acetyloxy)-6-{2-[2-(2-{[(benzyloxy)-carbonyl]amino}ethoxy)ethoxy]ethoxy}oxan-2-yl]methyl acetate 3 (50 g, 81.5 mmol) in MeOH (200 mL, 0.407 M, 4 Vols) was added TFA acid (6.24 mL, 81.5 mmol) and 10% palladium on carbon (2.5 g, 5% wt/wt). The solution was gently sparged with hydrogen for 1 hour then vigorously stirred for 16 hours under a hydrogen atmosphere. Upon completion, the solution was sparged with nitrogen then filtered through celite and concentrated in-vacuo to give compound 4 (44.6 g, 92.2%).

d. Synthesis of Compound 5

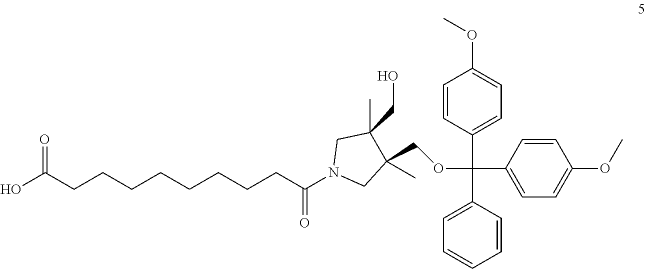 [SMILES: COc1ccc(C(OC[C@@]2(C)CN(C(=O)CCCCCCCCC(=O)O)C[C@@]2(C)CO)(c2ccccc2)c2ccc(OC)cc2)cc1]

Compound 5 was prepared as described in International Patent Application Publication Number WO 2017/117326 A1.

e. Synthesis of Compound 6

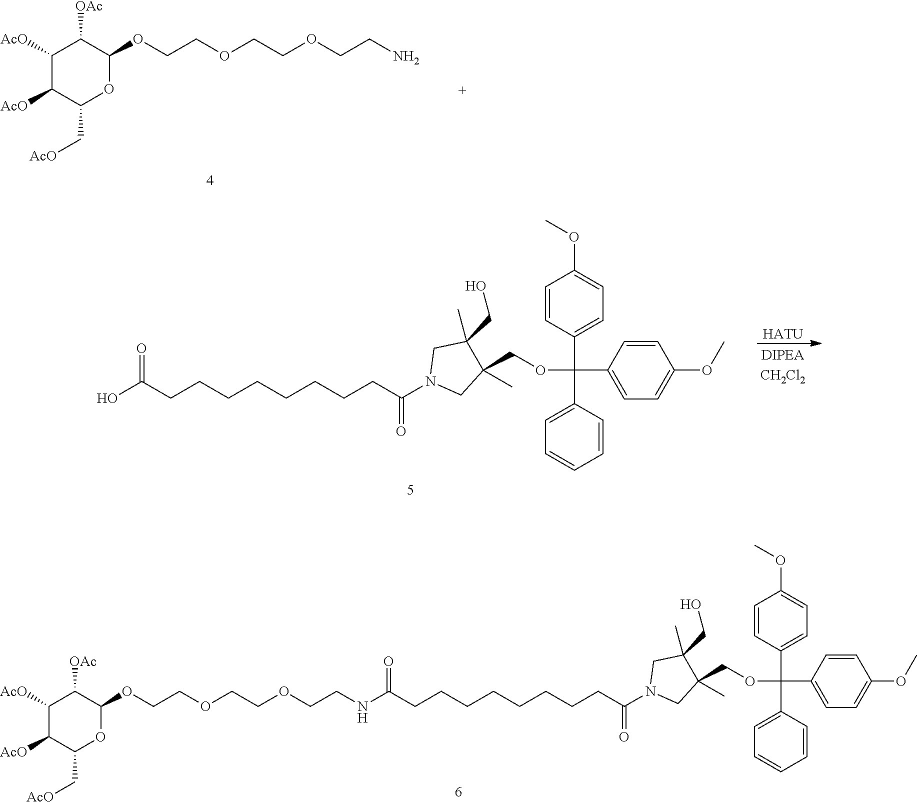

To a solution of compound 4 (1 g, 1.69 mmol), HATU (0.77 g, 2.0 mmol) and DIPEA (0.9 mL, 5.1 mmol) in DCM (15 mL) was added compound 5 (1.0 g, 1.5 mmol). The solution was stirred at room temperature for 16 hours. The solution was diluted with DCM (75 mL), washed with saturated NaHCO₃ (75 mL), dried (MgSO₄), filtered, and concentrated in-vacuo. Purification by automated column chromatography (0-15% MeOH/DCM) gave compound 6 (1.7 g, quant.).

f. Synthesis of Compound 7

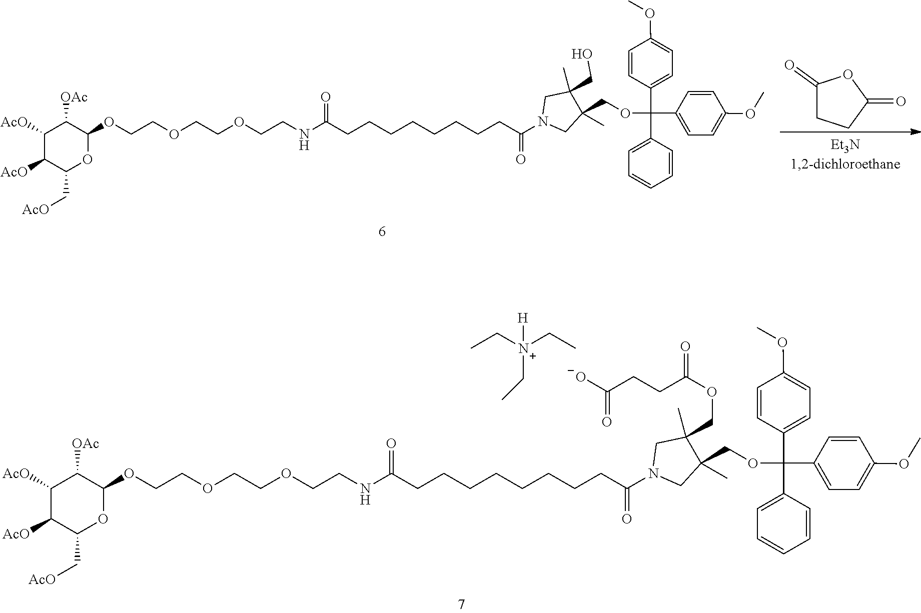

To a solution of compound 6 (1.7 g, 1.5 mmol) and TEA (2.2 mL, 15.4 mmol) in anhydrous DCE (50 mL) was added succinic anhydride (768 mg, 7.7 mmol). The solution was stirred at 75° C. for 2.5 hours then quenched with MeOH (1 mL) and stirred for an additional 1 hour. The reaction was diluted with DCM (50 mL), washed with saturated NaHCO₃ (2×50 mL), dried (MgSO₄), filtered, and concentrated in-vacuo to give compound 7 (2.1 g, quant.). The product was used without further purification. ¹H NMR (400 MHz, DMSO) δ 7.78 (q, J=5.3 Hz, 1H), 7.40-7.27 (m, 4H), 7.27-7.18 (m, 5H), 6.91-6.84 (m, 4H), 5.11 (dd, J=4.6, 1.5 Hz, 3H), 4.92 (dd, J=3.8, 1.5 Hz, 1H), 4.19-4.10 (m, 1H), 4.09-3.95 (m, 2H), 3.73 (s, 6H), 3.70-3.02 (m, 17H), 3.00-2.80 (m, 5H), 2.63 (s, 1H), 2.42-2.34 (m, 3H), 2.16-2.08 (m, 4H), 2.07-1.97 (m, 7H), 1.97-1.91 (m, 3H), 1.45 (q, J=7.0 Hz, 4H), 1.31-1.12 (m, 8H), 1.11-0.91 (m, 9H).

### Example 2. Preparation of Compound 17

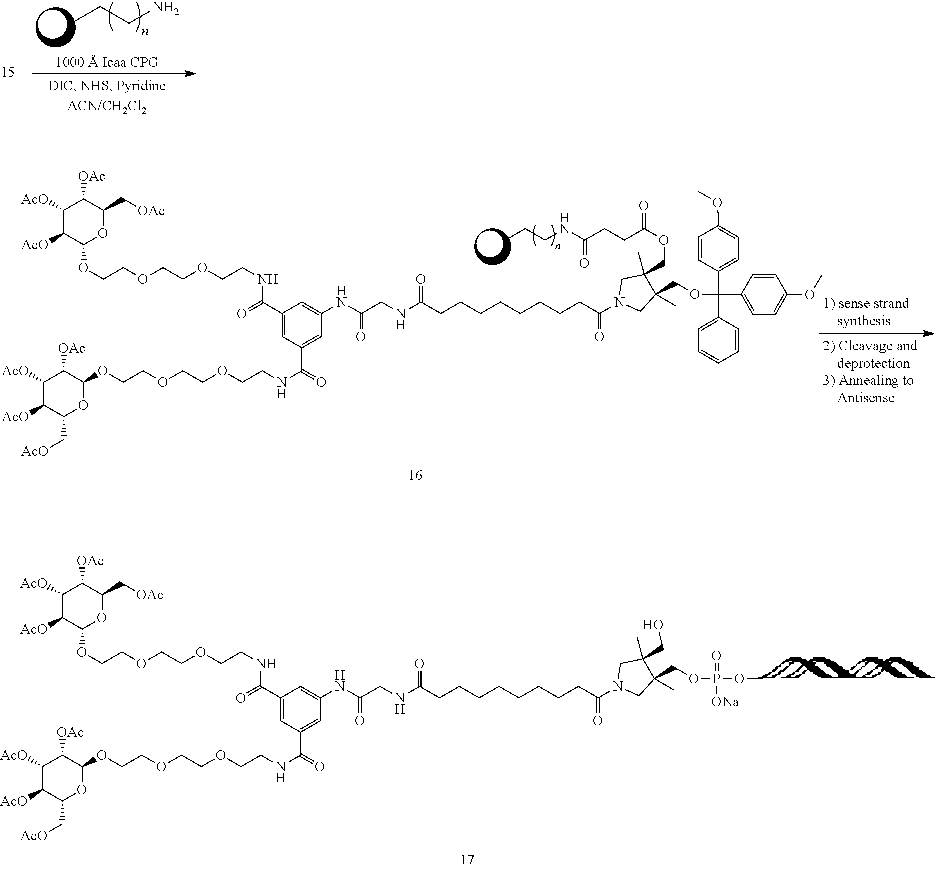

To a solution of compound 15 (1.0 g, 0.53 mmol), N-hydroxysuccinimide (12 mg, 0.11 mmol) and pyridine (0.13 mL, 1.6 mmol) in 1:1 MeCN/DCM (30 mL) was added 1000 Å 1caa CPG (long chain alkylamine, controlled pore glass, 7 g). The suspension was gently agitated at room temperature for 16 hours. Upon completion, the CPG was filtered, rinsed with DCM, air dried then suspended in a solution of 10% acetic anhydride, 5% N-methylimidazole and 5% pyridine in THE (30 mL). After 2 hours, the capping solution was filtered and the CPG rinsed with THF (50 mL), MeCN (50 mL) and DCM (50 mL) then dried under high vacuum for 16 hours to give 7.2 g loaded CPG. The succinate 15 loading efficiency was 36 μmol/g (determined by standard UV/Vis DMT assay @504 nm). The resulting bivalent mannose CPG solid support was employed in automated oligonucleotide synthesis using standard procedures. Nucleotide cleavage and deprotection, with concurrent mannose acetate deprotection, afforded the 3′ conjugated, bivalent mannose sense strand. After purification by dual HPLC, the corresponding antisense strand was annealed to form a siRNA duplex 17.

The intermediate Compound 15 was prepared from Compound 4 as described below.

a. Synthesis of Compound 10

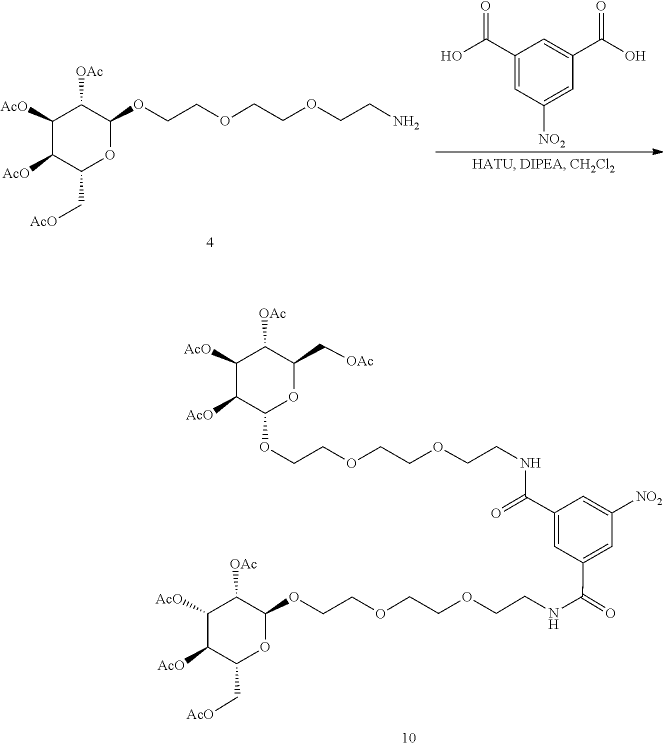 [SMILES: CC(=O)OC[C@H]1O[C@H](OCCOCCOCCN)[C@@H](OC(C)=O)[C@H](OC(C)=O)[C@@H]1OC(C)=O ; O=C(O)c1cc(C(=O)O)cc([N+](=O)[O-])c1 ; CC(=O)OC[C@H]1O[C@H](OCCOCCOCCNC(=O)c2cc(C(=O)NCCOCCOCCO[C@H]3O[C@H](COC(C)=O)[C@@H](OC(C)=O)[C@H](OC(C)=O)[C@@H]3OC(C)=O)cc([N+](=O)[O-])c2)[C@@H](OC(C)=O)[C@@H](OC(C)=O)[C@@H]1OC(C)=O]

A solution of 5-nitrobenzene-1,3-dicarboxylic acid (6.35 g, 30.0 mmol), compound 4 (44.6 g, 75.2 mmol), DIPEA (26.3 mL, 150.3 mmol) and HATU (25.2 g, 66.1 mmol) in DCM (500 mL) was stirred at room temperature for 16 hours. Upon completion, the reaction mixture was diluted with DCM (100 mL), washed with saturated NaHCO₃ (150 mL), dried (MgSO₄), filtered and concentrated in-vacuo. Purification by automated column chromatography (0-15% MeOH/DCM) gave compound 10 (46.5 g, quant.)

b. Synthesis of Compound 11

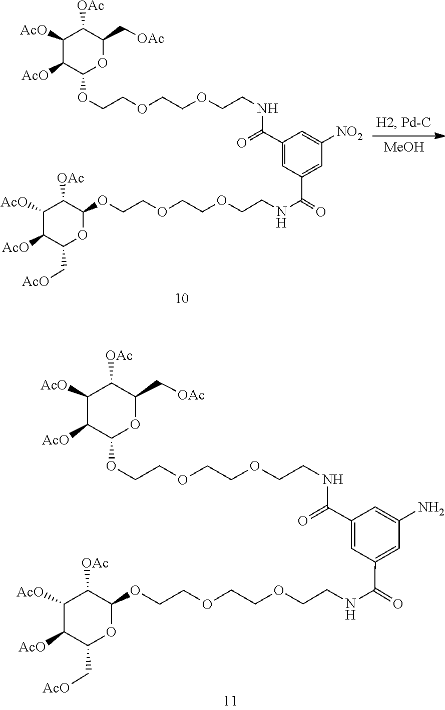 [SMILES: "MeOH" ; CC(=O)OC[C@H]1O[C@H](OCCOCCOCCNC(=O)c2cc(C(=O)NCCOCCOCCO[C@H]3O[C@H](COC(C)=O)[C@@H](OC(C)=O)[C@H](OC(C)=O)[C@@H]3OC(C)=O)cc([N+](=O)[O-])c2)[C@@H](OC(C)=O)[C@@H](OC(C)=O)[C@@H]1OC(C)=O ; CC(=O)OC[C@H]1O[C@H](OCCOCCOCCNC(=O)c2cc(N)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O]

To a solution of compound 10 (40.0 g, 35.3 mmol) in MeOH (250 mL) was added 10% palladium on carbon (2.0 g, 5% wt/wt). The solution was gently sparged with hydrogen for 1 hour then vigorously stirred at room temperature for 16 hours under a hydrogen atmosphere. Upon completion, the solution was sparged with nitrogen, filtered through celite and concentrated in-vacuo. The crude product was purified by automated column chromatography (0-10% MeOH/DCM) to give compound 11 (32 g, 82%).

c. Synthesis of Compound 12

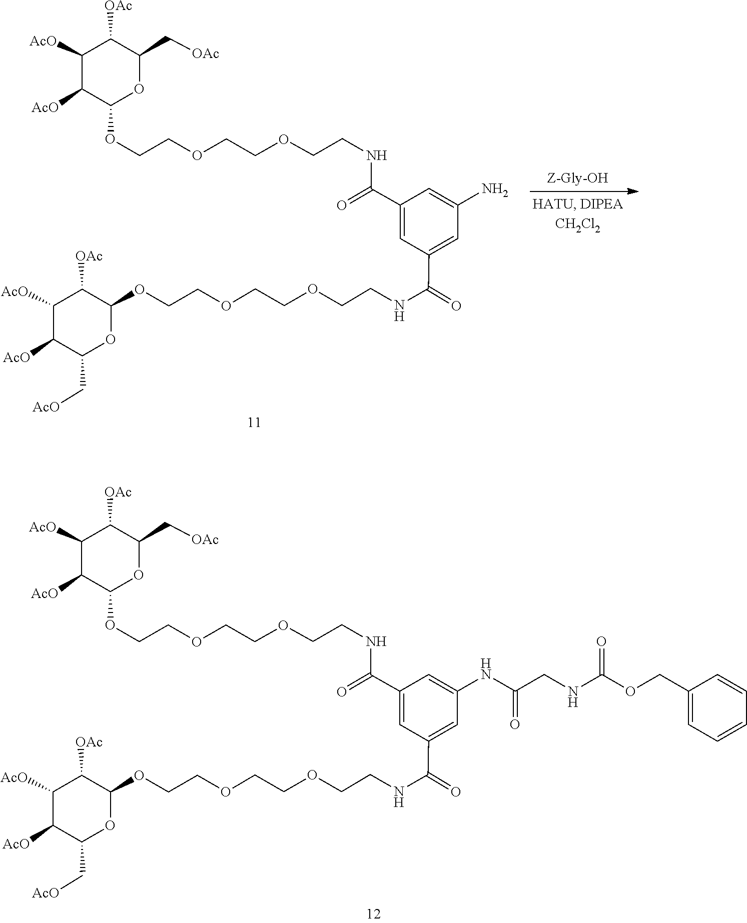 [SMILES: CC(=O)OC[C@H]1O[C@H](OCCOCCOCCNC(=O)c2cc(N)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O ; CC(=O)OC[C@H]1O[C@H](OCCOCCOCCNC(=O)c2cc(NC(=O)CNC(=O)OCc3ccccc3)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O]

A solution of compound 11 (32.0 g, 29.0 mmol), Z-Gly-OH (7.28 g, 34.8 mmol), HATU (14.3 g, 37.7 mmol) and DIPEA (20.3 mL, 116.0 mmol) in DCM (300 mL) was stirred for at room temperature for 2 hours. The reaction mixture was washed with saturated NaHCO₃ solution (200 mL), dried (MgSO₄), filtered and concentrated in-vacuo. The residue was purified by automated column chromatography (0-5% MeOH/DCM) to give compound 12 (31.0 g, 83%).

d. Synthesis of Compound 13

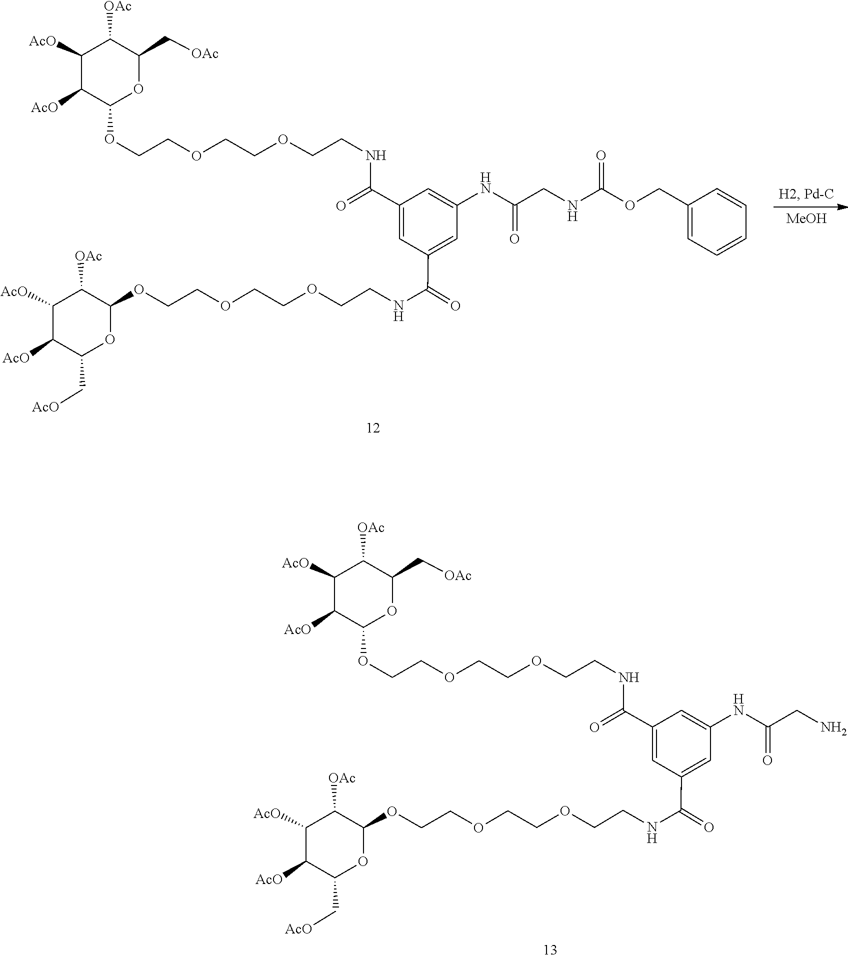 [SMILES: "MeOH" ; CC(=O)OC[C@H]1O[C@H](OCCOCCOCCNC(=O)c2cc(NC(=O)CNC(=O)OCc3ccccc3)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O ; CC(=O)OC[C@H]1O[C@H](OCCOCCOCCNC(=O)c2cc(NC(=O)CN)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O]

To a solution of compound 12 (31.0 g, 23.9 mmol) and TFA (1.83 mL, 23.9 mmol) in MeOH (250 mL) was added 10% palladium on Carbon (2.0 g). The solution was gently sparged with hydrogen for 1 hour then vigorously stirred at room temperature under a hydrogen atmosphere for 16 hours. The solution was sparged with nitrogen, filtered through celite and concentrated in-vacuo. The crude product was purified by automated column chromatography (5-15% MeOH/DCM) to give compound 13 (16.5 g, 54%).

e. Synthesis of Compound 14

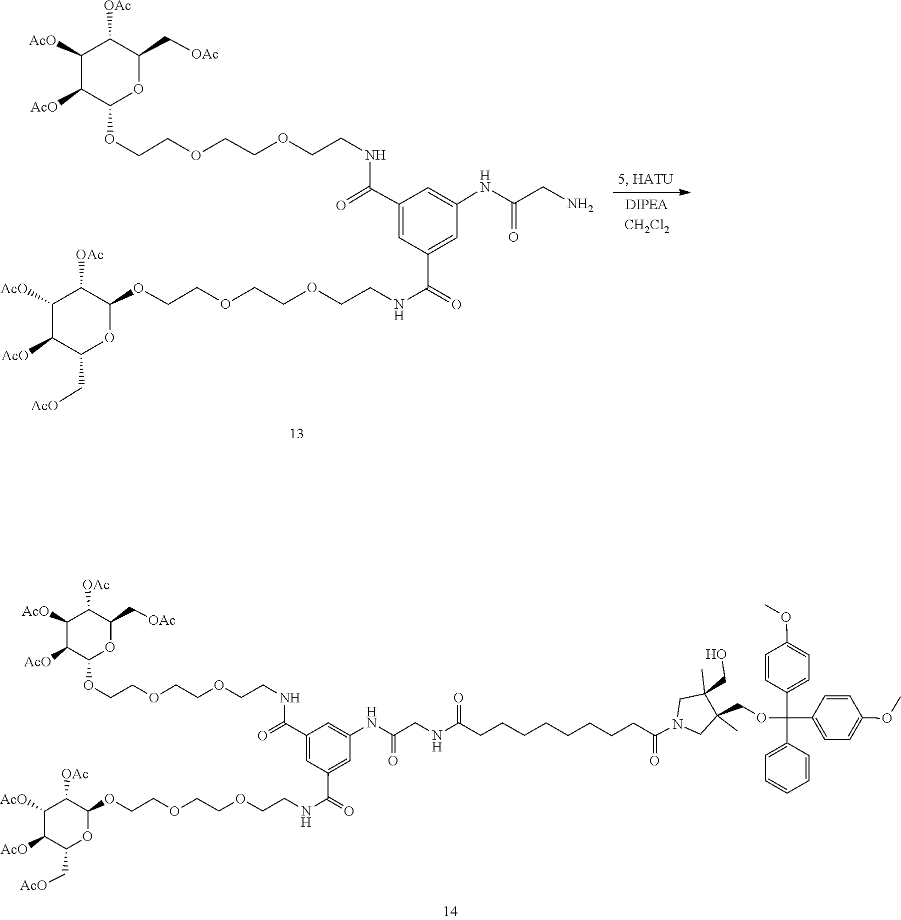 [SMILES: CC(=O)OC[C@H]1O[C@H](OCCOCCOCCNC(=O)c2cc(NC(=O)CN)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O ; COc1ccc(C(OC[C@@]2(C)CN(C(=O)CCCCCCCCC(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)C[C@@]2(C)CO)(c2ccccc2)c2ccc(OC)cc2)cc1]

To a solution of compound 13 (1.0 g, 0.8 mmol), HATU (358 mg, 0.9 mmol) and DIPEA (0.4 mL, 2.4 mmol) in DCM (25 mL) was added 5 (511 mg, 0.8 mmol). The solution was stirred at room temperature for 16 hours then diluted with DCM (75 mL), washed saturated NaHCO₃ (75 mL), dried (MgSO₄), filtered and concentrated in-vacuo. Purification by automated column chromatography (0-15% MeOH/DCM) gave compound 14 (1.3 g, 93%). ¹H NMR (400 MHz, DMSO) δ 10.19 (s, 1H), 8.52 (t, J=5.6 Hz, 2H), 8.13 (dd, J=6.0, 2.6 Hz, 2H), 7.94 (s, 1H), 7.39-7.18 (m, 9H), 6.92-6.84 (m, 4H), 5.16-5.05 (m, 5H), 4.94-4.89 (m, 2H), 4.53 (dt, J=16.3, 5.0 Hz, 1H), 4.19-4.10 (m, 2H), 4.09-3.95 (m, 4H), 3.87 (d, J=5.8 Hz, 2H), 3.73 (s, 6H), 3.67-3.50 (m, 19H), 3.50-3.33 (m, 5H), 3.32 (s, 6H), 3.28-2.84 (m, 6H), 2.20-2.05 (m, 8H), 2.02 (d, J=1.1 Hz, 12H), 1.93 (s, 6H), 1.56-1.39 (m, 1H), 1.30-1.19 (m, 13H), 1.08 (d, J=14.1 Hz, 3H), 0.93 (s, 3H).

f. Synthesis of Compound 15

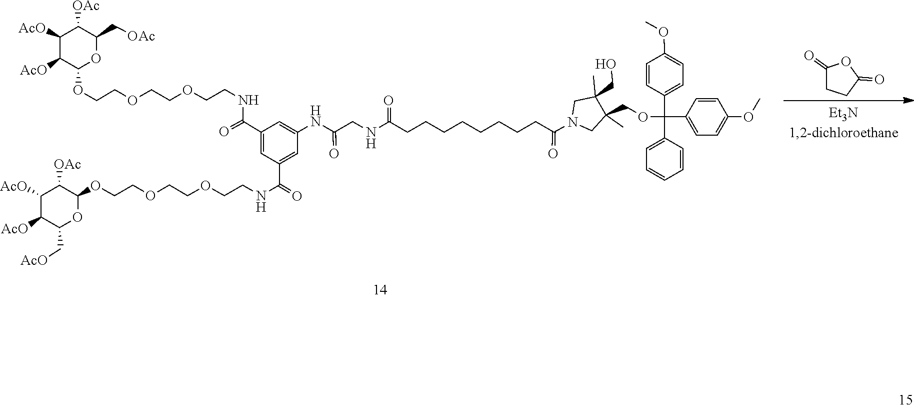 [SMILES: COc1ccc(C(OC[C@@]2(C)CN(C(=O)CCCCCCCCC(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)C[C@@]2(C)CO)(c2ccccc2)c2ccc(OC)cc2)cc1 ; O=C1CCC(=O)O1]

To a solution of compound 14 (1.3 g, 0.73 mmol) and TEA (1.02 mL, 7.3 mmol) in anhydrous DCE (25 mL) was added succinic anhydride (363 mg, 3.6 mmol). The solution was stirred at 75° C. for 3 hours then quenched with MeOH (1 mL) and stirred for 15 mins. The reaction mixture was diluted with DCM (50 mL), washed with saturated NaHCO₃ sodium bicarbonate (2×50 mL), dried (MgSO₄), filtered, and concentrated in-vacuo to give compound 15 (1.0 g, 73%). The product was used without purification.

### Example 3. Preparation of Compound 23

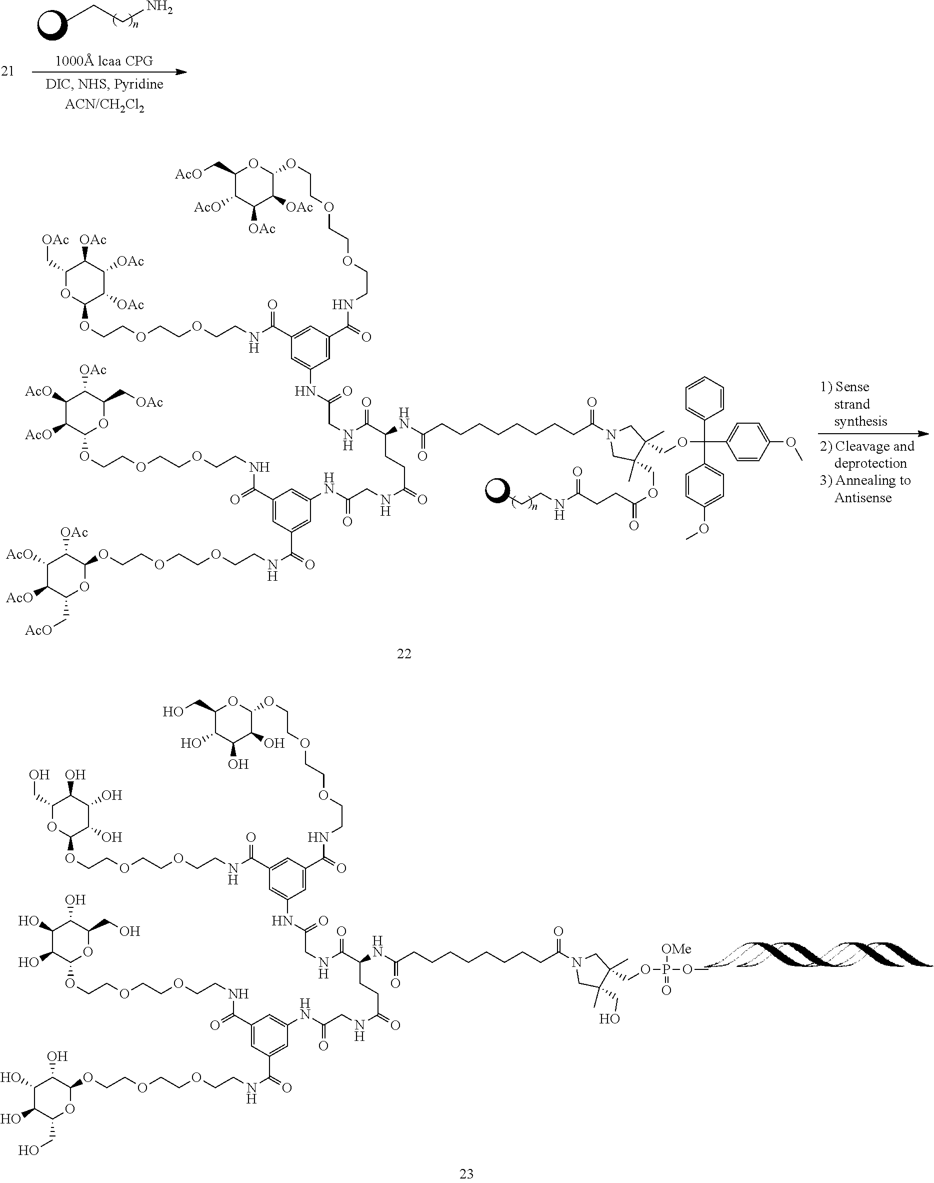

To a solution of compound 21 (740 mg, 0.24 mmol), N-hydroxysuccinimide (5.5 mg, 0.05 mmol) and pyridine (58 μL, 0.71 mmol) in 1:1 MeCN/DCM (20 mL) was added 1000 Å 1caa CPG (long chain alkylamine controlled pore glass, 4.5 g). The suspension was gently agitated at room temperature for 16 hours. Upon completion, the CPG was filtered, rinsed with DCM, air dried and suspended in a solution of 10% acetic anhydride, 5% N-methylimidazole and 5% pyridine in THE (30 mL). After 2 hours, the suspension was filtered and the remaining CPG rinsed with THF (50 mL), MeCN (50 mL), DCM (50 mL) and dried under high vacuum to afford 4.8 g. The succinate 21 loading efficiency was 23 μmol/g (determined by standard UV/Vis DMT assay @504 nm). The resulting tetra-valent mannose CPG solid support was employed in automated oligonucleotide synthesis using standard procedures. Nucleotide cleavage and deprotection, with concurrent mannose acetate deprotection, afforded the 3′ conjugated tetra-valent mannose sense strand. After purification by dual HPLC, the corresponding antisense strand was annealed to form a siRNA duplex 23.

The intermediate Compound 21 was prepared from Compound 13 as described below.

a. Synthesis of Compound 18

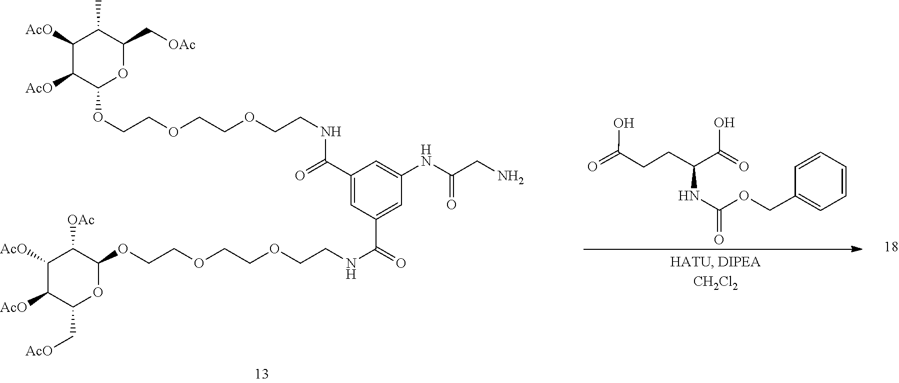 [SMILES: CC(=O)OC[C@H]1O[C@H](OCCOCCOCCNC(=O)c2cc(NC(=O)CN)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1C ; O=C(O)CC[C@H](NC(=O)OCc1ccccc1)C(=O)O]

A solution of compound 13 (1.1 g, 0.9 mmol), (2S)-2-{[(benzyloxy)carbonyl]-amino}pentanedioic acid (111 mg, 0.4 mmol), HATU (360 mg, 1.0 mmol) and DIPEA (0.35 mL, 2.0 mmol) in DCM (25 mL) was stirred at room temperature for 16 hours. Upon completion, the reaction was diluted with DCM (50 mL), washed with saturated NaHCO₃ (50 mL), dried (MgSO₄), filtered, and concentrated in vacuo. Purification by automated column chromatography (0-15% MeOH/DCM) gave compound 18 (920 mg, 91%).

b. Synthesis of Compound 19

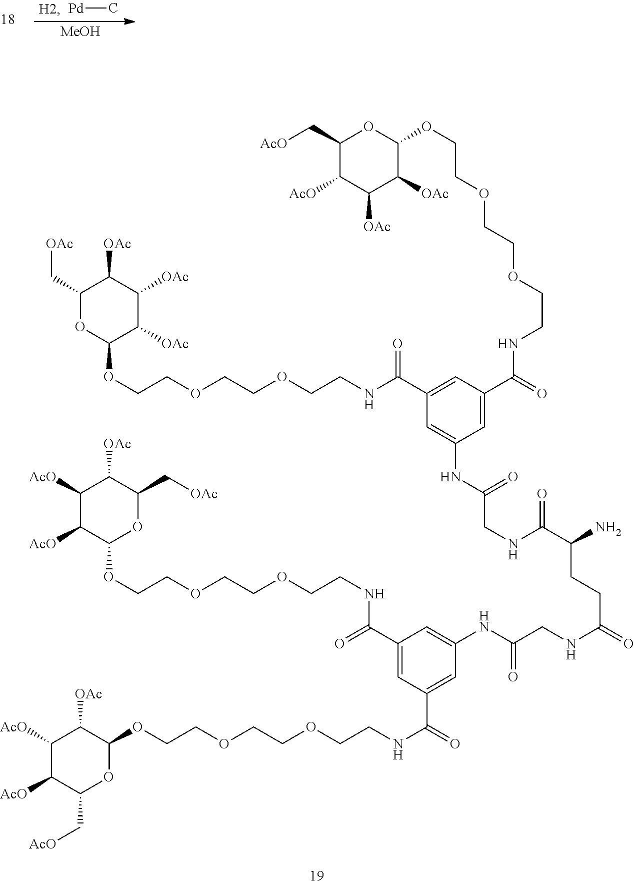

A solution of compound 18 (920 mg, 0.36 mmol), 10% palladium on carbon wet (100 mg) and TFA (28 μL, 0.36 mmol) in MeOH (75 mL) was stirred vigorously at room temperature for 16 hours under an atmosphere of hydrogen gas. The solution was filtered through celite and concentrated to give compound 19 as a colorless solid (720 mg, 83%).

c. Synthesis of Compound 20

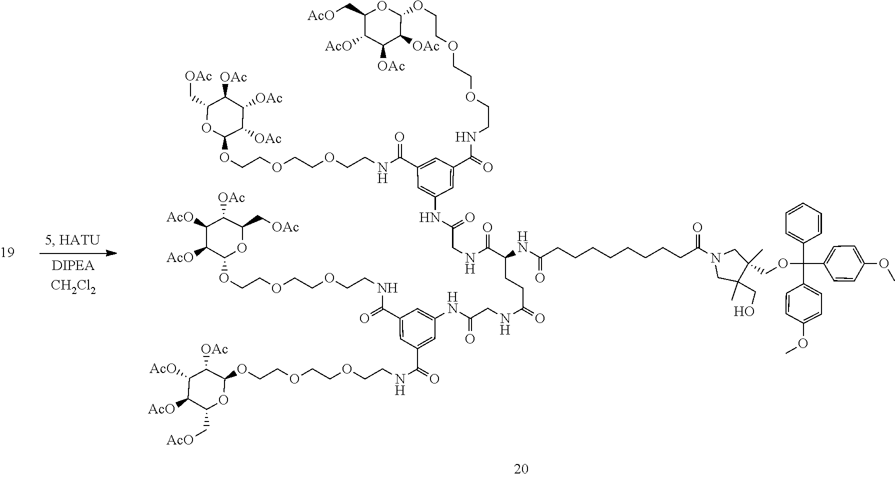 [SMILES: COc1ccc(C(OC[C@@]2(C)CN(C(=O)CCCCCCCCC(=O)N[C@@H](CCC(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)C(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)CC2(C)CO)(c2ccccc2)c2ccc(OC)cc2)cc1]

A solution of compound 19 (700 mg, 0.29 mmol), 5 (187 mg, 0.29 mmol), HATU (164 mg, 0.43 mmol) and DIPEA (151 μL, 0.86 mmol) in DCM (50 mL) was stirred at room temperature for 16 hours. Upon completion, the reaction was diluted with DCM (50 mL), washed (saturated NaHCO₃ (50 mL)), dried (MgSO₄), filtered, and concentrated in-vacuo. Purification by automated column chromatography (0-15% MeOH/DCM) gave compound 20 (770 mg, 87%).

d. Synthesis of Compound 21

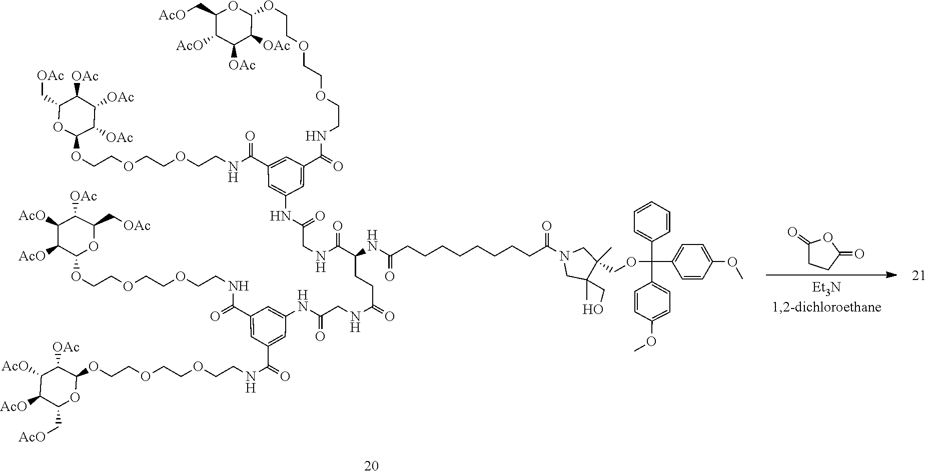 [SMILES: COc1ccc(C(OC[C@@]2(C)CN(C(=O)CCCCCCCCC(=O)N[C@@H](CCC(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)C(=O)NCC(=O)Nc3cc(C(=O)NCCOCCOCCO[C@H]4O[C@H](COC(C)=O)[C@@H](OC(C)=O)[C@H](OC(C)=O)[C@@H]4OC(C)=O)cc(C(=O)NCCOCCOCCO[C@H]4O[C@H](COC(C)=O)[C@@H](OC(C)=O)[C@H](OC(C)=O)[C@@H]4OC(C)=O)c3)CC2(C)CO)(c2ccccc2)c2ccc(OC)cc2)cc1 ; O=C1CCC(=O)O1]

To a solution of compound 20 (770 mg, 0.25 mmol) and TEA (354 μL, 2.5 mmol) in anhydrous DCE (20 mL) was added succinic anhydride (227 mg, 2.3 mmol). The solution was stirred at 75° C. for 3 hours then quenched with MeOH (1 mL) with stirring for 15 mins. The reaction mixture was diluted DCM (50 mL), washed with saturated NaHCO₃ (2×50 mL), dried (MgSO₄), filtered, and concentrated in-vacuo to give compound 21 (740 mg, 93%). The product was without further purification. ¹H NMR (400 MHz, MeOD) δ 8.22 (s, 2H), 8.17 (s, 2H), 7.95 (s, 2H), 7.44-7.37 (m, 2H), 7.33-7.15 (m, 8H), 6.85 (dt, J=9.1, 2.4 Hz, 4H), 5.29-5.18 (m, 12H), 4.86 (s, 4H), 4.38 (t, J=7.0 Hz, 1H), 4.22 (dd, J=12.3, 5.0 Hz, 4H), 4.13-4.00 (m, 12H), 3.86-3.74 (m, 14H), 3.73-3.61 (m, 39H), 3.61-3.55 (m, 9H), 3.43-3.19 (m, 4H), 3.16 (q, J=7.3 Hz, 2H), 3.12-2.97 (m, 1H), 2.53-2.41 (m, 6H), 2.33-2.18 (m, 4H), 2.12 (s, 12H), 2.04 (s, 12H), 2.03 (s, 12H), 1.94 (s, 12H), 1.65-1.50 (m, 4H), 1.39-1.33 (m, 2H), 1.32-1.21 (m, 8H), 1.21-1.12 (m, 3H), 1.12-1.03 (m, 3H).

### Example 4. Preparation of Compound 32

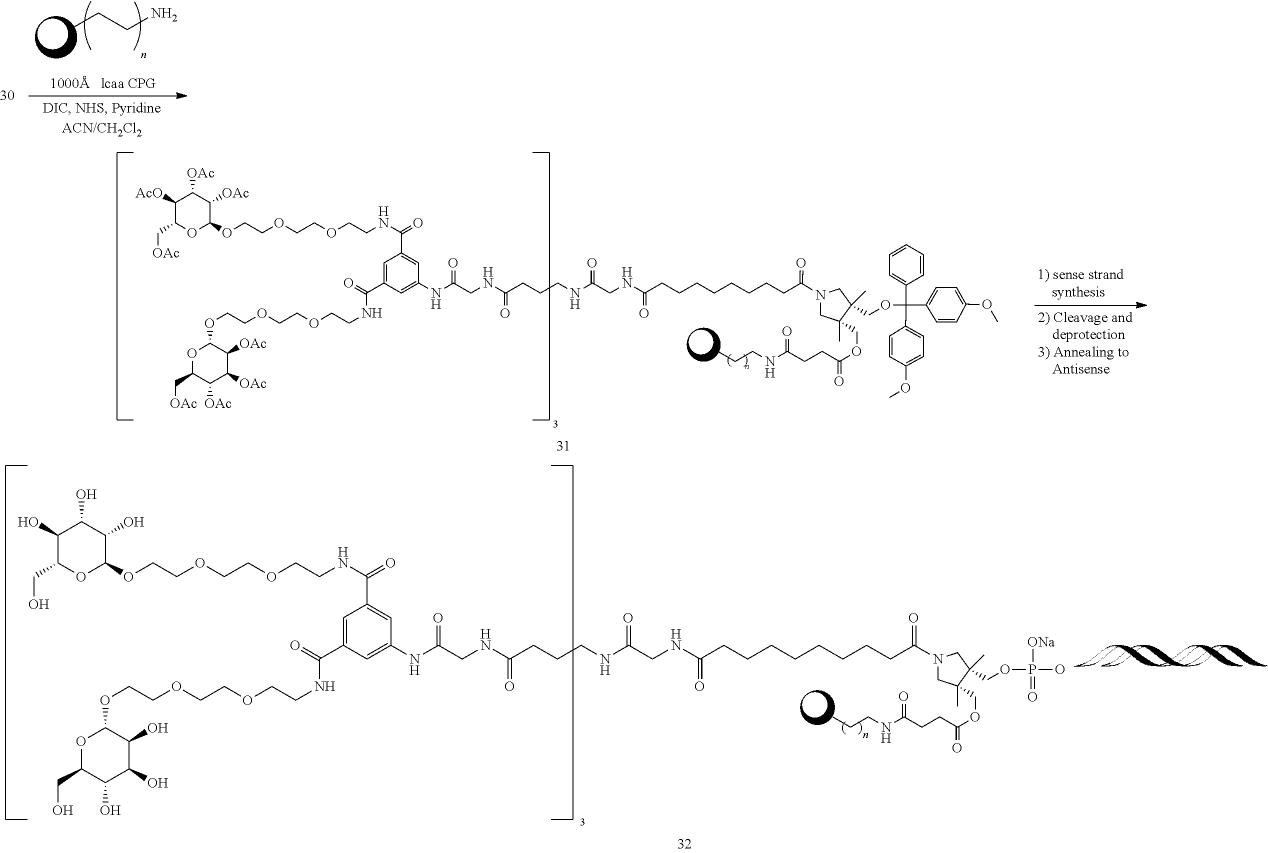

To a solution of compound 30 (11.5 g, 0.24 mmol), N-hydroxysuccinimide (59 mg, 0.52 mmol) and pyridine (625 μL, 0.7 mmol) in 1:1 MeCN/DCM (400 mL) was added 1000 Å 1caa CPG (long chain alkylamine, controlled pore glass, 92 g). The suspension was agitated at room temperature for 16 hours. Upon completion, the CPG was filtered, rinsed with DCM, air dried and suspended in a solution of 10% acetic anhydride, 5% N-methylimidazole and 5% pyridine in THE (400 mL) at RT. After 2 hours, the suspension filtered and the remaining CPG was rinsed with THE (250 mL), MeCN (250 mL), DCM (250 mL) and dried under high vacuum to give 103 g. The succinate 21 loading efficiency was 15 μmol/g (determined by standard UV/Vis DMT assay @504 nm). The resulting hexa-valent mannose CPG solid support was employed in automated oligonucleotide synthesis using standard procedures. Nucleotide cleavage and deprotection, with concurrent mannose acetate deprotection, afforded the 3′ conjugated hexa-valent mannose sense strand. After purification by dual HPLC, the corresponding antisense strand was annealed to form a siRNA duplex 32.

The intermediate Compound 30 was prepared from Compound 24 as described below.

a. Synthesis of Compound 25

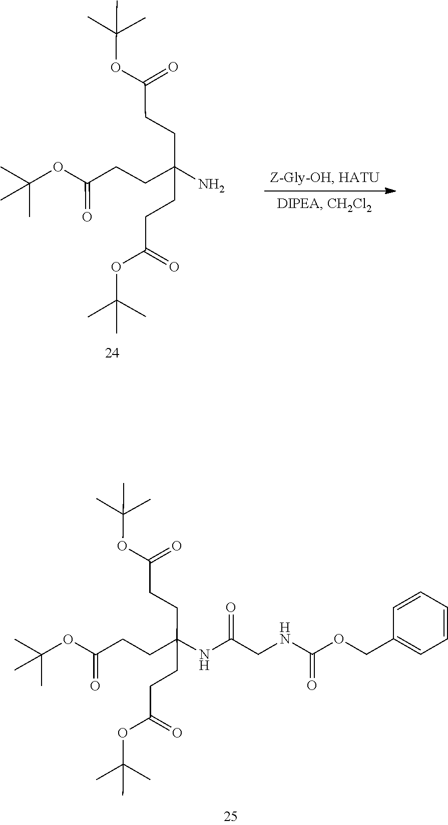 [SMILES: CC(C)(C)OC(=O)CCC(N)(CCC(=O)OC(C)(C)C)CCC(=O)OC(C)(C)C ; CC(C)(C)OC(=O)CCC(CCC(=O)OC(C)(C)C)(CCC(=O)OC(C)(C)C)NC(=O)CNC(=O)OCc1ccccc1]

To a solution of Z-Gly-OH (5.5 g, 26.5 mmol), DIPEA (9.3 mL, 53.0 mmol) and HATU (10.5 g, 27.7 mmol) in DCM (100 mL) was added 1,7-di-tert-butyl 4-amino-4-[3-(tert-butoxy)-3-oxopropyl]heptanedioate 24 (10.0 g, 24.1 mmol). The reaction was stirred at room temperature for 16 hours, washed (NaHCO₃ (100 mL)), dried (MgSO₄) filtered and concentrated in vacuo. Purification by automated column chromatography (0-50% EtOAc/hexane) gave 1,7-di-tert-butyl 4-(2-{[(benzyloxy)carbonyl]amino}acetamido)-4-[3-(tert-butoxy)-3-oxopropyl]heptanedioate 25 as a colorless solid (8.0 g, 55%).

b. Synthesis of Compound 26

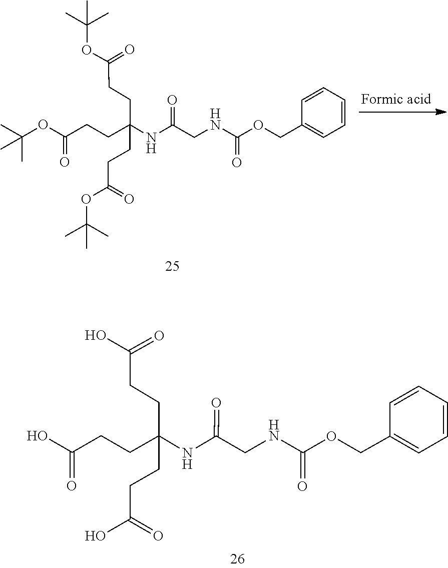 [SMILES: CC(C)(C)OC(=O)CCC(CCC(=O)OC(C)(C)C)(CCC(=O)OC(C)(C)C)NC(=O)CNC(=O)OCc1ccccc1 ; O=C(O)CCC(CCC(=O)O)(CCC(=O)O)NC(=O)CNC(=O)OCc1ccccc1]

A solution of compound 25 (8.0 g, 13.2 mmol) in formic acid (50 mL) was stirred at room temperature for 16 hours. Upon completion, the solution was concentrated in vacuo, precipitated from methyl tert-butyl ether (50 mL), filtered, and dried under vacuum to give compound 26 (5.4 g, 93%).

c. Synthesis of Compound 27

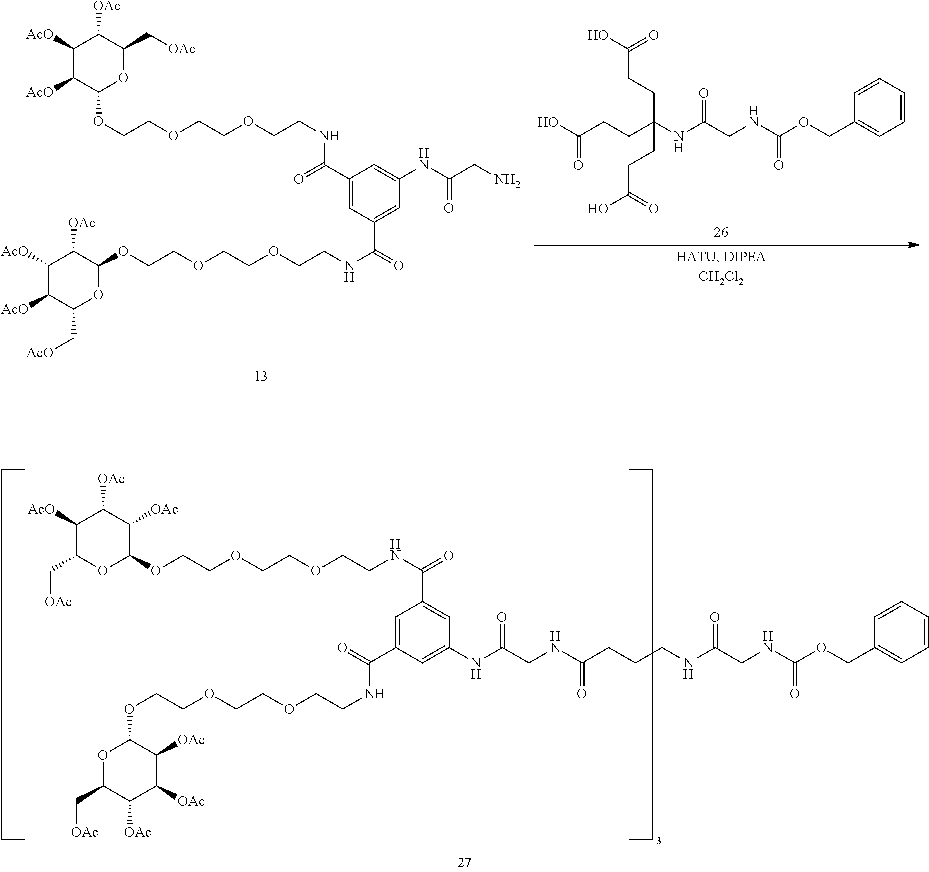

To a solution of compound 13 (15.0 g, 11.76 mmol, 3.3 equiv.), 4-(2-{[(benzyloxy)carbonyl]amino}acetamido)-4-(2-carboxyethyl)heptanedioic acid 26 (1.56 g, 3.6 mmol) and DIPEA (6.2 mL, 35.6 mmol) in DCM (100 mL) was added HATU (5.4 g, 14.3 mmol). The solution was stirred for at room temperature for 16 hours. Upon completion, the solution was diluted (DCM 100 mL), washed (saturated NaHCO₃ (2×100 mL)), dried (MgSO₄, filtered, and concentrated in-vacuo. Purification by automated column chromatography (0-10% MeOH/DCM) gave compound 27 (12.3 g, 89%).

d. Synthesis of Compound 28

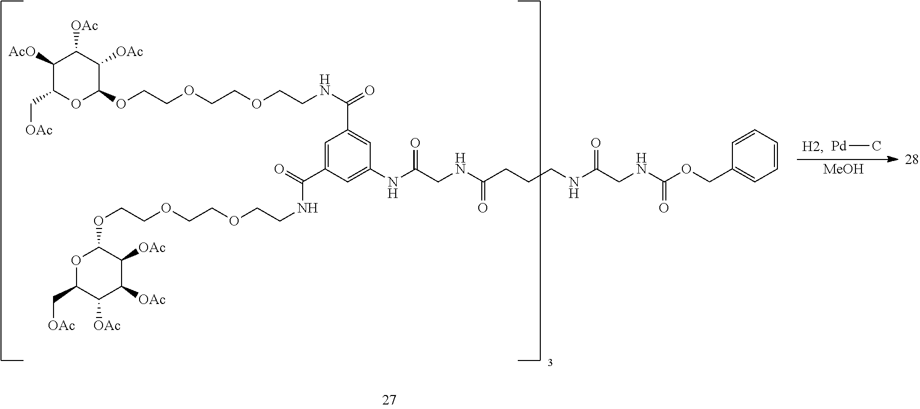

A solution of compound 27 (13.0 g, 3.4 mmol), 10% palladium on carbon (1 g) and TFA acid (257 μL, 3.4 mmol) in MeOH (150 mL) was vigorously stirred at room temperature under an atmosphere of hydrogen gas for 16 hours. The solution was filtered through celite and concentrated to give compound 28 (720 mg, 83%). The product was used without further purification.

e. Synthesis of Compound 29

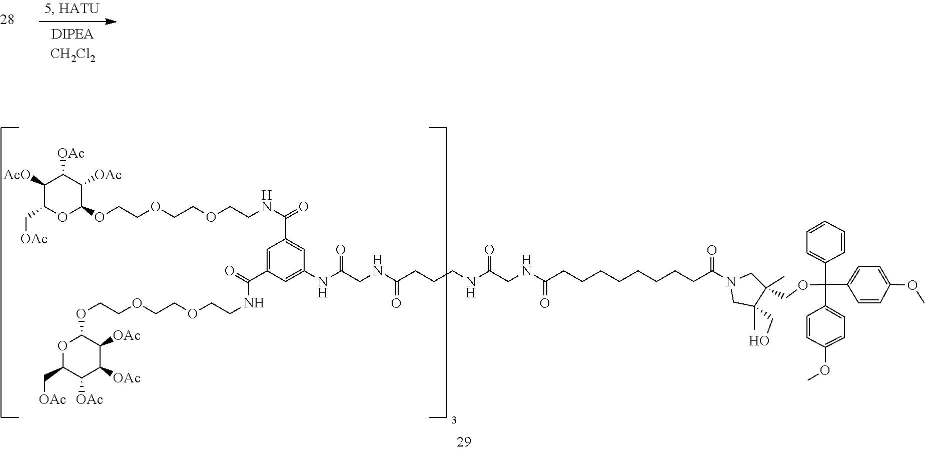

To a solution of compound 5 (2.1 g, 3.2 mmol), HATU (1.47 g, 3.86 mmol) and DIPEA (1.66 g, 12.9 mmol) in DCM (100 mL) was added compound 28 (12.0 g, 3.2 mmol). The reaction was stirred at room temperature for 16 hours, diluted with DCM (150 mL), washed (saturated NaHCO₃ (2×150 mL)), dried (MgSO₄), filtered, and concentrated in-vacuo. Purification by automated chromatography (0-20% MeOH/DCM) gave compound 29 (12.5 g, 92%). ¹H NMR (400 MHz, DMSO) δ 10.23 (s, 3H), 8.54 (t, J=5.7 Hz, 5H), 8.23-8.13 (m, 8H), 7.99-7.89 (m, 3H), 7.37-7.28 (m, 4H), 7.25-7.19 (m, 5H), 6.92-6.85 (m, 4H), 5.15-5.07 (m, 16H), 4.92 (s, 6H), 4.16 (dd, J=12.2, 5.1 Hz, 6H), 4.09-3.96 (m, 12H), 3.90 (d, J=5.8 Hz, 5H), 3.73 (d, J=8.2 Hz, 14H), 3.67-3.50 (m, 52H), 3.43 (q, J=5.8 Hz, 10H), 2.20-2.07 (m, 8H), 2.11 (s, 18H), 2.02 (d, J=1.3 Hz, 36H), 1.96-1.85 (m, 4H), 1.94 (s, 18H), 1.45 (s, 4H), 1.22 (t, J=13.2 Hz, 14H), 1.09 (d, J=14.3 Hz, 3H), 0.94 (s, 3H).

f. Synthesis of Compound 30

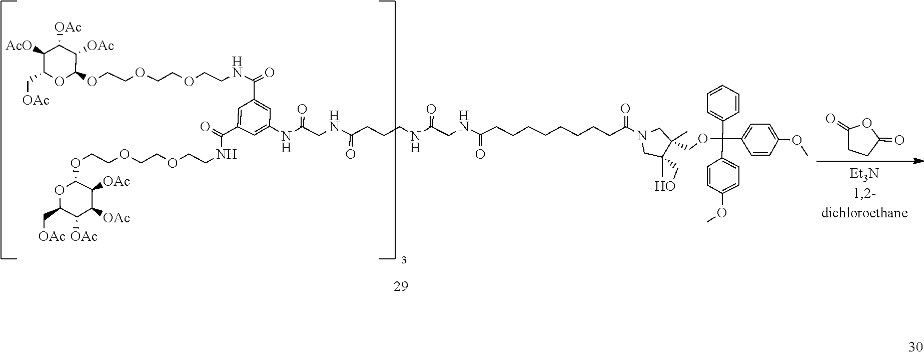

To a solution of compound 29 (12.5 g, 2.9 mmol) in DCE (100 mL) was added TEA (4.14 mL, 29.4 mmol) and succinic anhydride (1.47 g, 14.7 mmol). The solution was stirred at 75° C. for 3 hours, quenched with MeOH (10 mL) with stirring for 15 mins. The reaction was diluted with DCM (150 mL), washed with saturated NaHCO₃ (2×150 mL), dried (MgSO₄), filtered, and concentrated in-vacuo to give compound 30 (12.5 g, 97%). The product was used without purification. ¹H NMR (400 MHz, DMSO) δ 10.25 (q, J=7.7 Hz, 3H), 8.54 (q, J=5.4 Hz, 6H), 8.27-8.12 (m, 6H), 7.94 (s, 4H), 7.39-7.26 (m, 8H), 7.26-7.18 (m, 6H), 6.91-6.83 (m, 5H), 5.16-5.05 (m, 18H), 4.91 (s, 6H), 4.15 (dd, J=12.2, 5.1 Hz, 6H), 4.06 (d, 4H), 4.04-3.94 (m, 10H), 3.92-3.84 (m, 6H), 3.76-3.66 (m, 14H), 3.66-3.48 (m, 61H), 3.49-3.37 (m, 13H), 3.38-2.80 (m, 5H), 2.54 (q, 4H), 2.39-2.34 (m, 5H), 2.21-2.05 (m, 31H), 2.01 (d, J=1.3 Hz, 39H), 1.93 (s, 18H), 1.96-1.82 (m, 5H), 1.44 (s, 6H), 1.20 (t, J=9.8 Hz, 14H), 1.12-1.04 (m, 3H), 1.01-0.90 (m, 13H).

### Comparative Example 5. Preparation of Compound 33

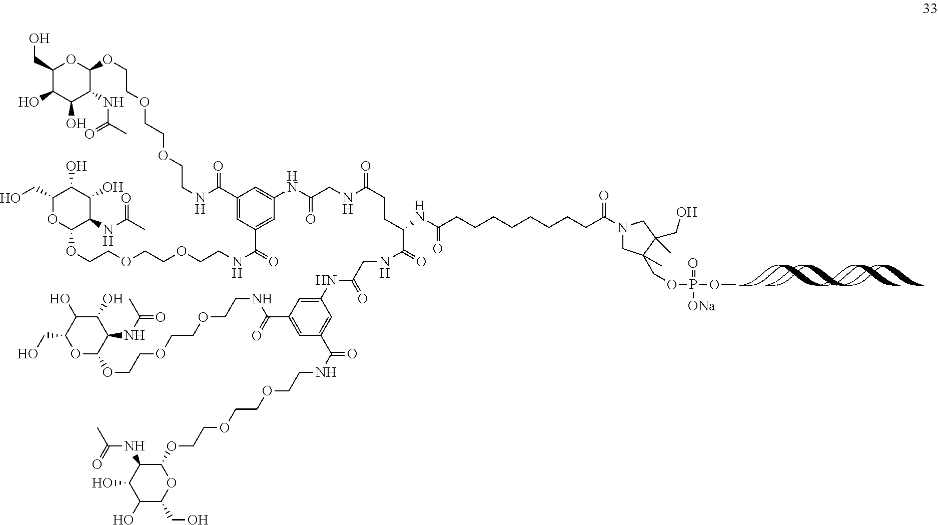

Compound 33 was prepared using a method analogous to the method used to prepare Compound 23, starting from tetra-valent GalNAc CPG (see WO 2017/117326 A1 for the preparation of GalNAc CPG)

### Example 6. Synthesis of Compound 35

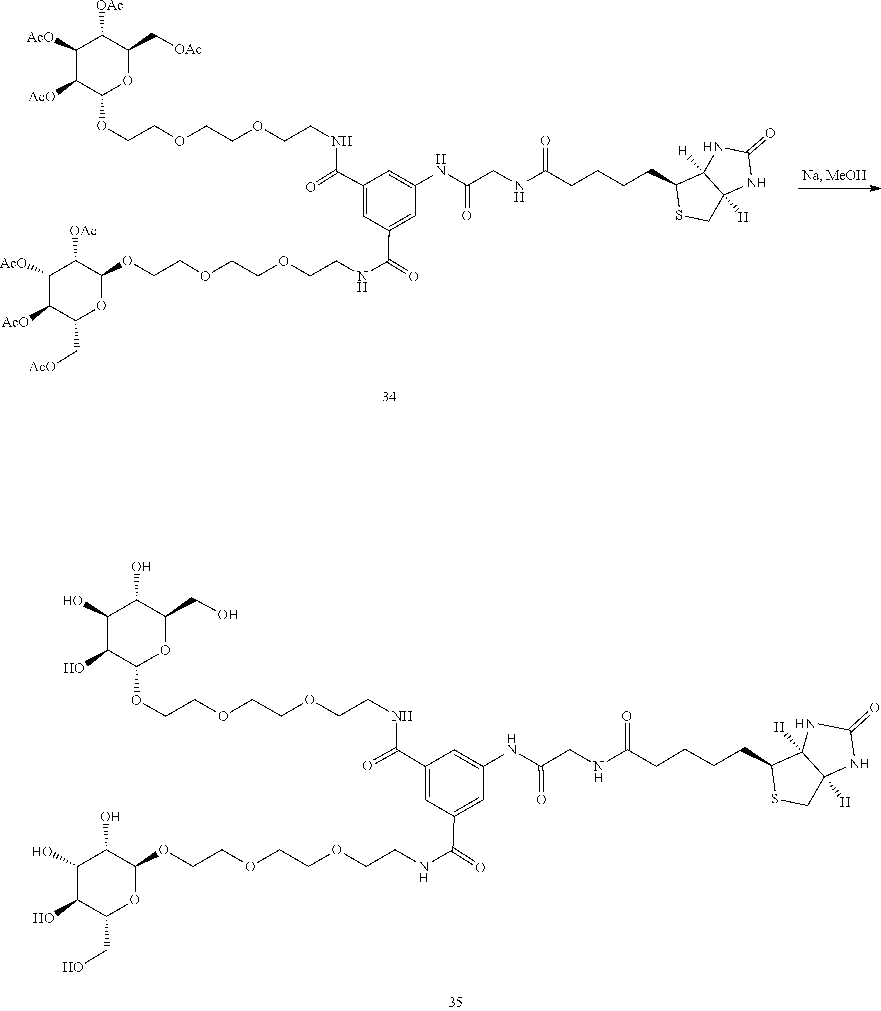 [SMILES: CC(=O)OC[C@H]1O[C@H](OCCOCCOCCNC(=O)c2cc(NC(=O)CNC(=O)CCCC[C@@H]3SC[C@@H]4NC(=O)N[C@@H]43)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O ; O=C(CCCC[C@@H]1SC[C@@H]2NC(=O)N[C@@H]21)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1]

Sodium (5 mg, 0.22 mmol) was added to a solution of compound 34 (400 mg, 0.29 mmol) in anhydrous MeOH (20 mL) and stirred at room temperature for 16 hours. Upon completion, the solution was concentrated in-vacuo and the residue purified by preparative HPLC (Agilent Zorbax SB-C18, PN 870150-902, 21.2 mm×150 mm, Sum; 0-30% acetonitrile/water; 20 mL/min). The product was lyophilized to give compound 35. MS (ESI) m/z: [M+Na]+ Calcd for C₄₄H₇₀N₆NaO₂₁S, 1073.42; Found 1073.42.

The intermediate Compound 34 was prepared from Compound 13 as described below.

a. Synthesis of Compound 34

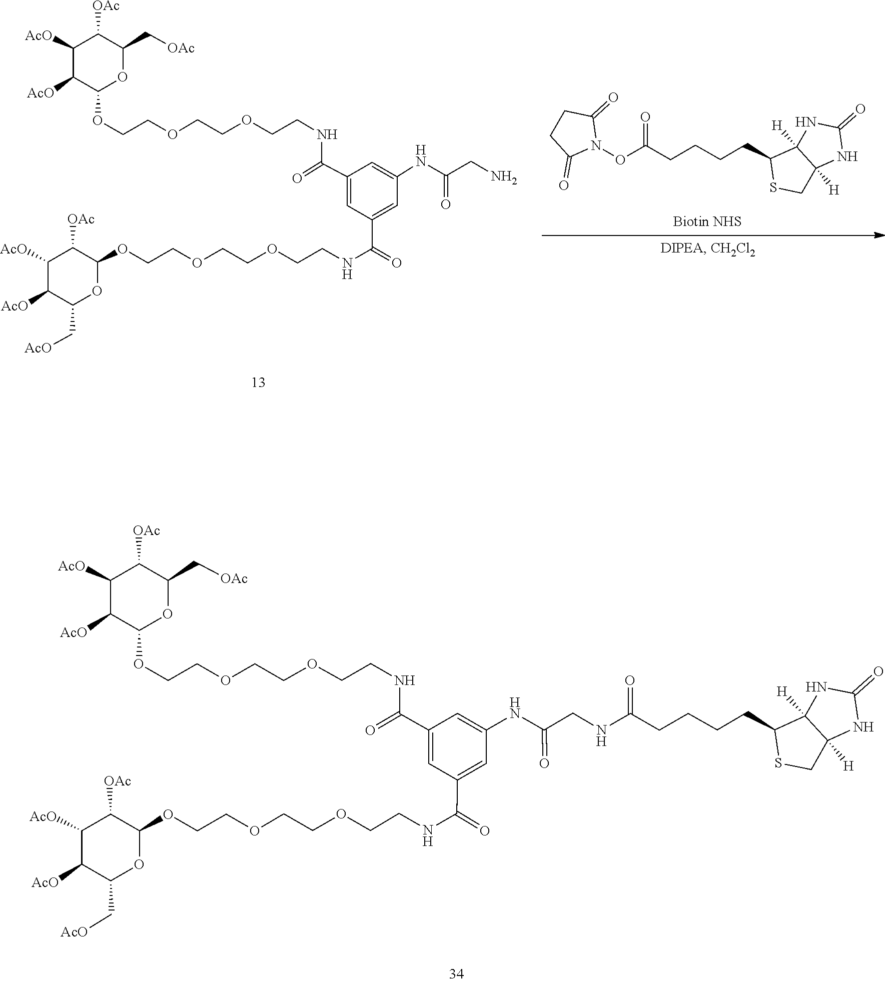 [SMILES: CC(=O)OC[C@H]1O[C@H](OCCOCCOCCNC(=O)c2cc(NC(=O)CN)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O ; O=C1N[C@H]2[C@H](CS[C@H]2CCCCC(=O)ON2C(=O)CCC2=O)N1 ; CC(=O)OC[C@H]1O[C@H](OCCOCCOCCNC(=O)c2cc(NC(=O)CNC(=O)CCCC[C@@H]3SC[C@@H]4NC(=O)N[C@@H]43)cc(C(=O)NCCOCCOCCO[C@H]3O[C@H](COC(C)=O)[C@@H](OC(C)=O)[C@H](OC(C)=O)[C@@H]3OC(C)=O)c2)[C@@H](OC(C)=O)[C@@H](OC(C)=O)[C@@H]1OC(C)=O]

A solution of compound 13 (500 mg, 0.43 mmol), Biotin NHS (176 mg, 0.52 mmol) and DIPEA (188 μL, 1.1 mmol) in DCM (10 mL) was stirred at room temperature for 16 hours. The reaction was diluted with DCM (25 mL) washed with saturated NaHCO₃ solution (25 mL), dried (MgSO₄), filtered, and concentrated in-vacuo. Purification by column chromatography (0-15% MeOH/DCM) gave compound 35 (405 mg, 67.8%).

### Example 7. Synthesis of Compound 36

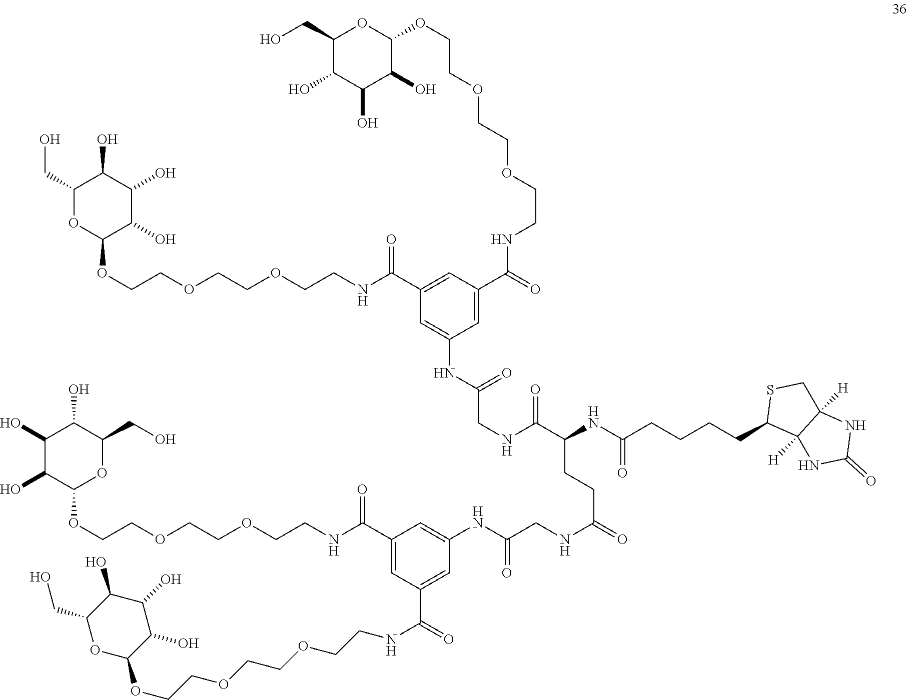 [SMILES: O=C(CC[C@H](NC(=O)CCCC[C@H]1SC[C@H]2NC(=O)N[C@H]21)C(=O)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1]

Compound 36 was synthesized from Compound 19 using a method analogous to the method used to prepare Compound 35.

### Example 8. Synthesis of Compound 37

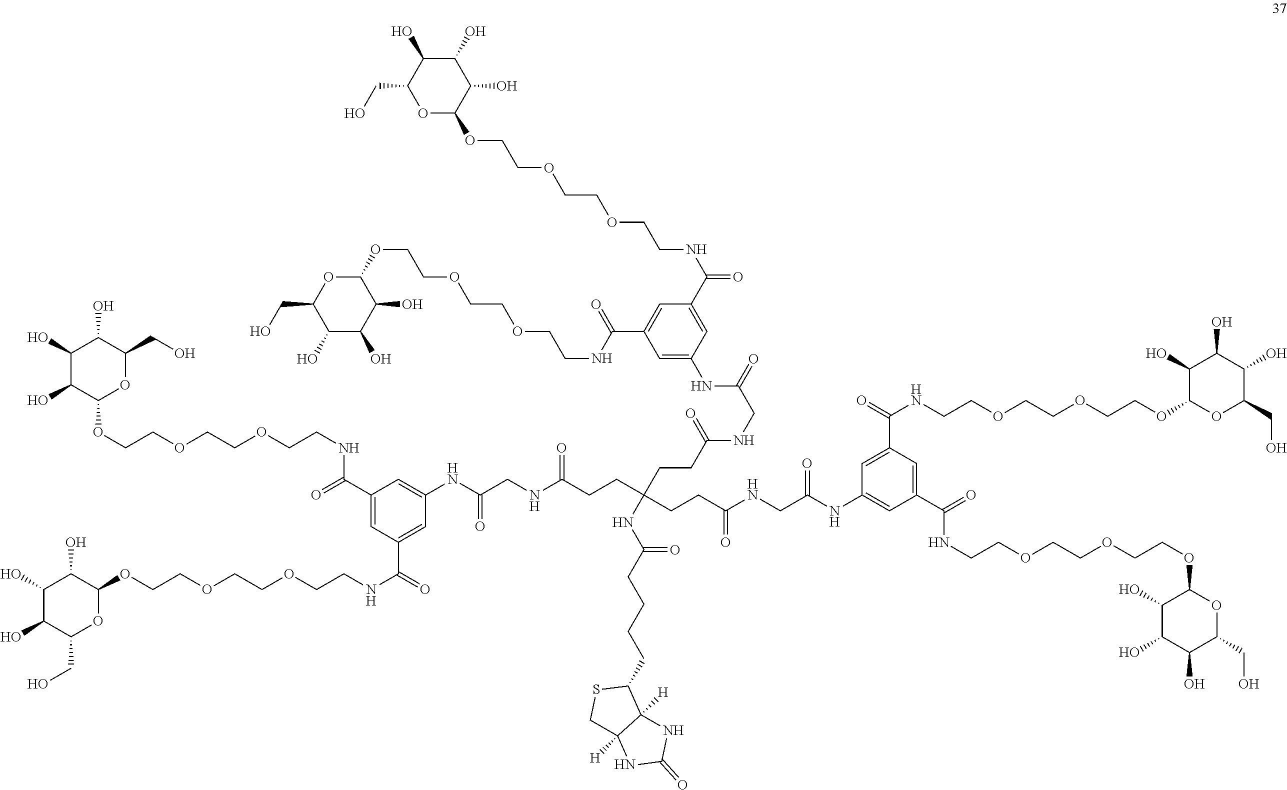 [SMILES: O=C(CCC(CCC(=O)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1)(CCC(=O)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1)NC(=O)CCCC[C@H]1SC[C@@H]2NC(=O)N[C@@H]21)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1]

Compound 37 was synthesized from Compound 28 using a method analogous to the method used to prepare Compound 35.

### Example 9. Synthesis of Compound 38

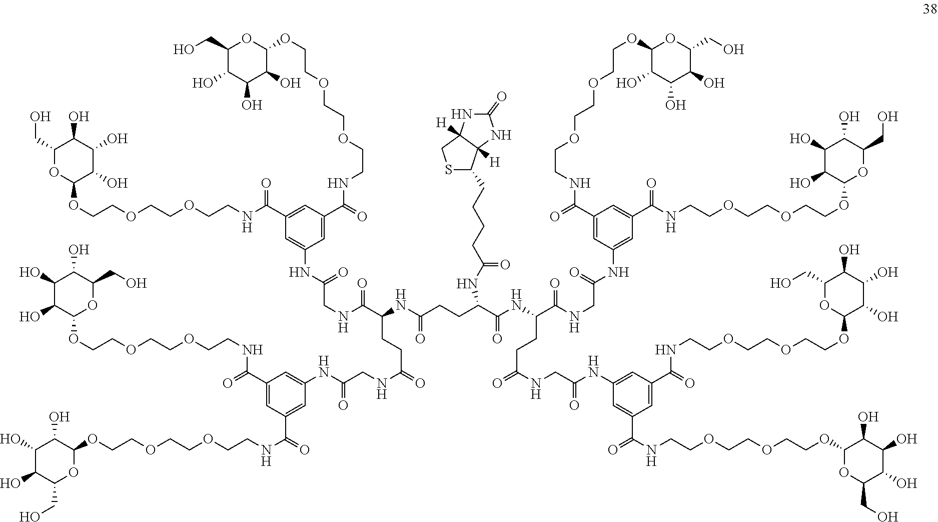

Compound 38 was synthesized using a method analogous to the method used to prepare Compound 37.

### Example 10. Synthesis of Compound 39

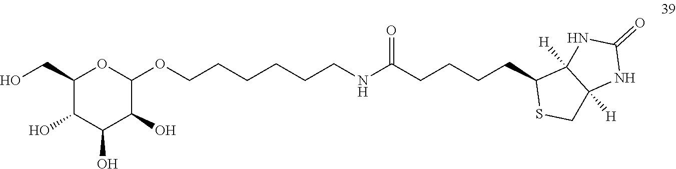 [SMILES: O=C(CCCC[C@@H]1SC[C@@H]2NC(=O)N[C@@H]21)NCCCCCCOC1O[C@H](CO)[C@@H](O)[C@H](O)[C@@H]1O]

Compound 39 was synthesized using a method analogous to the method used to prepare Compound 35.

The following siRNA materials were incorporated into Compounds 23, 32, and 33 as shown in Table 1.

TABLE 1
siRNA conjugates

| | | | Mass Analysis | |
|---|---|---|---|---|
| | | | (MALDI; Da.) | |
| siRNA | Sequence (5′-3′) | Seq. ID | Calc. | Found |
| 23a Cy3- | Sense strand: | SEQ ID | 9132.1 | 9130.5 |
| Tetra- | [Cy3]mCsmUmGmGfCmUfGfAfAmUmUmUmCmAmGmAmGmCsmA | NO: 1 | | |
| mannose | [23] | | | |
| siCD45 | Antisense strand: | SEQ ID | 6920.6 | 6920.2 |
| | mUsfGsmCmUmCfUmGfAfAmAmUmUmCfAmGfCmCmAmGsmUsmU | NO: 2 | | |
| 32a Hexa- | Sense strand: | SEQ ID | 9424.37 | 9425.93 |
| mannose- | mCsmUmGmGfCmUfGfAfAmUmUmUmCmAmGmAmGmCsmA[32] | NO: 3 | | |
| siCD45 | Antisense strand: | SEQ ID | 6920.6 | 6919.1 |
| | mUsfGsmCmUmCfUmGfAfAmAmUmUmCfAmGfCmCmAmGsmUsmU | NO: 4 | | |
| 32b Cy3- | Sense strand: | SEQ ID | 10055.2 | 10056.0 |
| hexa- | [Cy3]mCsmUmGmGfCmUfGfAfAmUmUmUmCmAmGmAmGmCsmA | NO: 5 | | |
| mannose- | [32] | | | |
| siCD45 | Antisense strand: | SEQ ID | 6920.7 | 6921.8 |
| | mUsfGsmCmUmCfUmGfAfAmAmUmUmCfAmGfCmCmAmGsmUsmU | NO: 6 | | |
| 32c | Sense strand: | SEQ ID | 10446.5 | 10448.0 |
| AF647- | [AF647)mCsmUmGmGfCmUfGfAfAmUmUmUmCmAmGmAmGmCsm | NO: 7 | | |
| hexa- | A[32] | | | |
| mannose- | Antisense strand: | SEQ ID | 6920.7 | 6919.1 |
| siCD45 | mUsfGsmCmUmCfUmGfAfAmAmUmUmCfAmGfCmCmAmGsmUsmU | NO: 8 | | |
| 32d Hexa- | Sense strand: | SEQ ID | 9986.7 | 9988.0 |
| mannose- | mGsmAsmUmUmCmUfCmAfGfGfAmCmUmUmCmUmUmAmUmUmA | NO: 9 | | |
| siMARV | [32] | | | |
| | Antisense strand: | SEQ ID | 7678.2 | 7679.2 |
| | mUsfAsmAmUmAfAmGfAfAmGmUmCmCfUmGfAmGmAmAmUmCs | NO: 10 | | |
| | mUsmA | | | |
| 33a Tetra- | Sense strand: | SEQ ID | 8624.5 | 8624.2 |
| GalNAc- | mCsmUmGmGfCmUfGfAfAmUmUmUmCmAmGmAmGmCsmA[33] | NO: 11 | | |
| siCD45 | Antisense strand: | SEQ ID | 6920.6 | 6919.1 |
| | mUsfGsmCmUmCfUmGfAfAmAmUmUmCfAmGfCmCmAmGsmUsmU | NO: 12 | | |
| 33b Cy3- | Sense strand: | SEQ ID | 8967.9 | 8966.3 |
| Tetra- | [Cy3]mCsmUmGmGfCmUfGfAfAmUmUmUmCmAmGmAmGmCsmA | NO: 13 | | |
| GalNAc- | [33] | | | |
| siCD45 | Antisense strand: | SEQ ID | 6920.6 | 6920.2 |
| | mUsfGsmCmUmCfUmGfAfAmAmUmUmCfAmGfCmCmAmGsmUsmU | NO: 14 | | |
| 33c Tetra- | Sense strand: | SEQ ID | 9186.9 | 9189.1 |
| GalNAc- | mGsmAsmUmUmCmUfCmAfGfGfAmCmUmUmCmUmUmAmUmUmA | NO: 15 | | |
| siMARV | [33] | | | |
| | Antisense strand: | SEQ ID | 7678.3 | 7678.3 |
| | mUsfAsmAmUmAfAmGfAfAmGmUmCmCfUmGfAmGmAmAmUmCs | NO: 16 | | |
| | mUsmA | | | |
| 32e Hexa- | Sense strand: | SEQ ID | 9408.4 | 9406.7 |
| mannose- | fUsmCsfCmUfAmUfGmAfCmUfGmUfAmGfAmUfUmUfUsmAsfU[32] | NO: 17 | | |
| siHprt1 | Antisense strand: | SEQ ID | 6946.6 | 6945.2 |
| | pmAsfUsmAfAmAfAmUfCmUfAmCfAmGfUmCfAmUfAmGfGmAsfAs | NO: 18 | | |
| | mU | | | |

2′-O-Methyl nucleotides are depicted as “m” + UPPER CASE; 2′-Fluoro nucleotides are depicted as “f” + UPPER CASE; Phosphorothioate linkers are depicted as “s”.

### Synthesis of Endosome Release Polymer

Endosome release polymers (Mannose-ERP 40 and GalNAc-ERP 41) were synthesized by Syngene International LTD using synthetic methodology analogous to that described in Prieve, M. G., Harvie, P., Monahan, S. D., Roy, D., Li, A. G., Blevins, T. L., Paschal, A. E., Waldheim, M., Bell, E. C., Galperin, A., Ella-Menye, J. R., et al. (2018). Targeted mRNA Therapy for Ornithine Transcarbamylase Deficiency. Mol Ther 26, 801-813. 10.1016/j.ymthe.2017.12.024. The structures of Mannose-ERP 40 and GalNAc-ERP 41 are presented below.

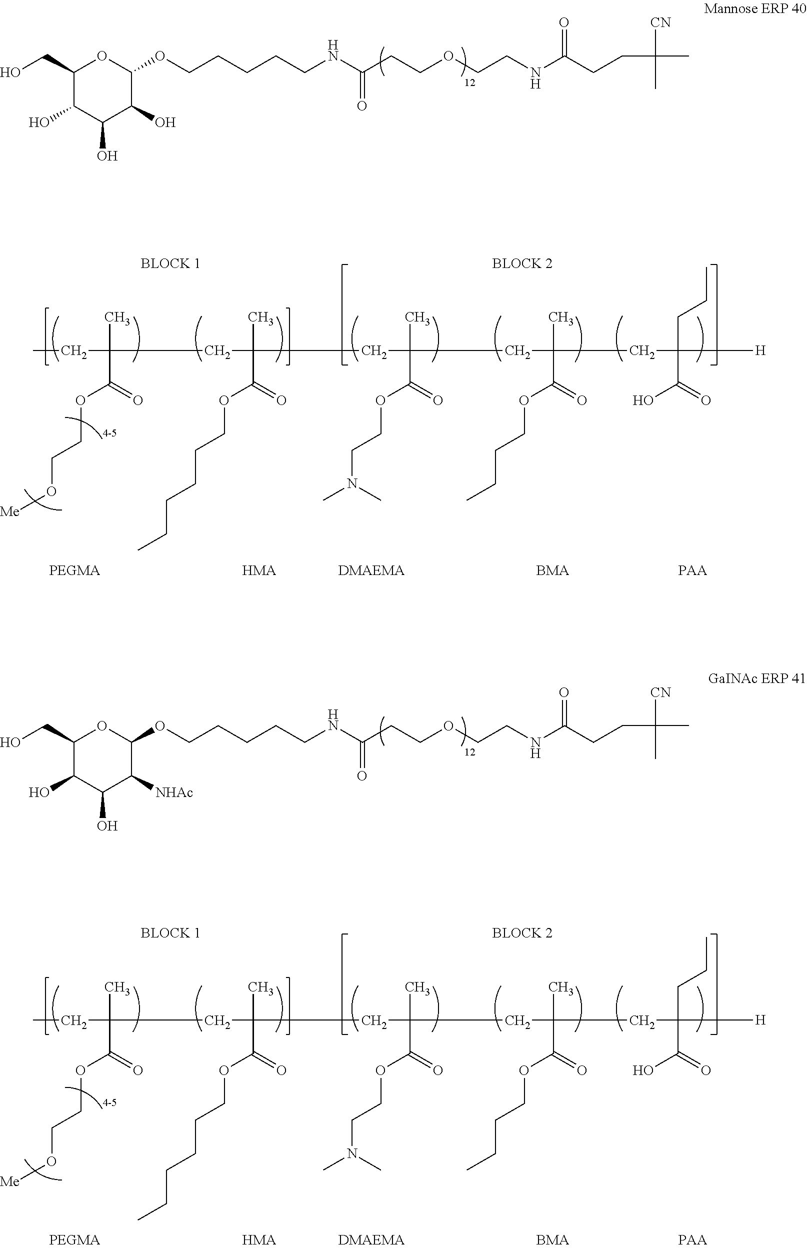

Block 1 MW 5.7 KDa 15% of Target value (4.85-6.56 KDa)
Block 2 MW 7.9 kDa 15% of target value (9.11-6.73 kDa)
Block 1 Monomer Incorporation
   PEGMA 75% (71-79%)
   HMA 25% (21-29%)
Block 2 Monomer Incorporation
   BMA 54% (47-57%)
   DMAEMA 35% (30-40%)
   PAA 12% (9-16%)

### Example 11. Human CD14+ Monocyte Derived M1 Macrophages Expresses Higher CD206 than M2

This example illustrates the expression level of CD206 in human CD14+ monocyte derived M1 and M2 macrophages. CD206 is the target receptor for mannose ligands. To identify an optimal in vitro model for testing mannose ligand binding, the expression level of CD206 in human CD14+ derived M1 and M2 macrophages, which are widely used in vitro macrophage cell models were compared.

### Materials and Methods

Differentiation of M1 and M2 Macrophages from Human CD14+ Monocytes

Human Buffy coat was purchased from BioIVT. CD14+ monocytes were isolated from buffy coat using the StraightFrom® Buffy Coat CD14 MicroBead Kit following the manufacturer's protocol. The isolated CD14 monocytes were differentiated to M1 and M2 macrophages following a previously published protocol (Macrophages-Springer New York_Humana Press (2018)). In brief, monocytes were cultured in RPMI medium containing 10 mM HEPES, 2 mM L-glutamine and 10% FBS. To trigger M1 macrophage differentiation, cells were stimulated with 50 ng/mL GM-CSF on Day 0 and 3 and further induced with 50 ng/mL IFN-γ on Day 6. For M2 macrophage differentiation, cells were treated with 50 ng/mL M-CSF on Day 0 and 3 and further induced with 20 ng/mL IL-4 in combination with 20 ng/mL IL-10 on Day 6. Differentiated M1 and M2 were tested for ligand binding on Day 7.

Characterization of CD206 Expression with Flow Cytometry

M1 and M2 macrophages derived from human CD14+ monocytes were resuspended in PBS containing 2 mM EDTA and seeded to sterile V bottom 96-well plates at 40000 cells/well. Cells were pelleted by centrifugation at 200 g for 5 minutes and treated with CD16/32 Fc receptor blocker at 5 μg/mL on ice for 5 min to reduce non-specific binding from antibodies. Mannose receptor CD206 was stained with BV510 conjugated mouse anti-human CD206 antibodies by incubation at 4° C. for 20 min. Cells were then washed with stain buffer twice and resuspended in stain buffer prior to FACS analysis.

### Flow Cytometry Analysis

Analyses were performed on a FACS-Canto II using the software FACS Diva. As a marker for viability, cells were stained with Live/Dead red. The forward scatter and side scatter gate were set to include all viable cells.

Approximately 10000-15000 cells were counted for each sample and the expression of CD206 was determined as increased intensity in BV510 detected with the blue lase channel. Cells stained with IgG isotype control was used for gating positive and negative cell populations. The % CD206+ in live cells and the CD206 BV510 MFI in live cells were calculated.

### Results

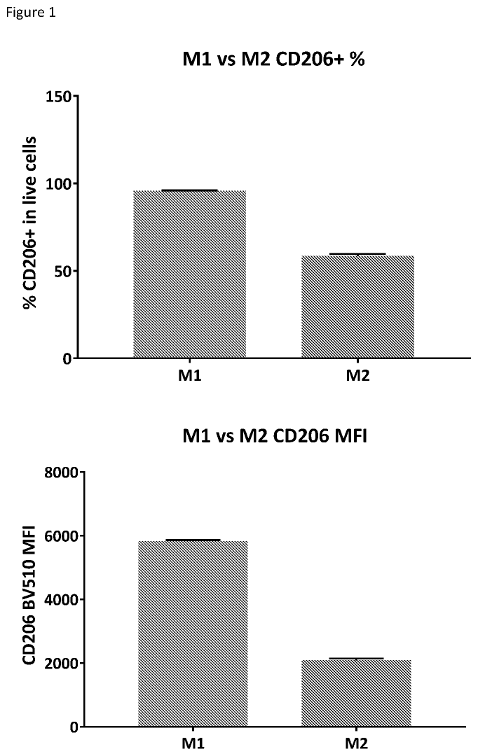

CD206 expression was detected in both M1 and M2 macrophages differentiated from human CD14+ monocytes (FIG. 1). M1 expressed higher level of CD206 than M2.

### Example 12. Binding of Monovalent and Multivalent Mannose Ligands by Human CD14+ Monocyte Derived M1 and M2 Macrophages In Vitro

This example illustrates that uptake of monovalent and multivalent mannose ligands in human CD14+ monocyte derived M1 and M2 macrophages in vitro. The confirmation of CD206 expression in M1 and M2 macrophage allowed the binding affinity of mannose ligands with different valences to be tested in these cell models.

### Materials and Methods

Differentiation of M1 and M2 Macrophages from Human CD14+ Monocytes

M1 and M2 macrophages were derived from human CD14+ monocytes as described in Example 11.

### Preparation of Biotin-Mannose/AF488-Streptavidin and Biotin-GalNAc/AF488-Streptavidin Complex

Biotinylated monovalent 39 or multivalent (di-35, tetra-36, hexa-37, Octa-38) mannose ligands were reconstituted in DMSO and functionalized by incubating with Alex Fluor 488-labelled streptavidin in Tyrode buffer (containing 10 mM HEPES, 5.6 mM glucose, 10 mM KCl, 35 mM NaCl, 0.4 mM MgCl, 1.0 mM CaCl2) and 0.1% BSA, pH 7.3) at 4° C. overnight at a molar ratio of 4.5:1 of biotinylated ligands to biotin binding sites.

### Binding of Biotin-Mannose/AF488-Streptavidin Complex

Human CD14+ monocytes derived M1 and M2 macrophages were washed with Dulbecco's phosphate buffered saline (DPBS) containing 2 mM EDTA and resuspended in ice-cold Tyrode buffer and seeded into sterile V-bottom 96-well plates (40000/well). The previously prepared biotin-mannose/AF488-streptavidin complex was diluted in Tyrode buffer and mixed with the seeded macrophages to reach final concentrations of 0.5, 0.1 and 0.02 μM (based on streptavidin molar concentration). Biotin-GalNAc/AF488-streptavidin complex was included as control treatment (GalNAc is a ligand for the ASGP receptor, which is not present on macrophages). The cells were incubated with the mannose ligand complexes at 4° C. for 1.5 hours. After incubation, the cells were washed three times with ice-cold DPBS to remove any unbound ligand complex. After centrifuging at 1200 RPM for 5 minutes, and the cell pellet was resuspended in stain buffer prior to FACS analysis.

### Flow Cytometry Analysis

Analyses were performed on a FACS-Canto II using the software FACS Diva. As a marker for viability, cells were stained with Live/Dead Red. The forward scatter and side scatter gate were set to include all viable cells.

Approximately 10000-15000 cells were counted for each sample and binding/uptake was determined as increased intensity in green fluorescence at 488 nm detected in the FL1 channel. The mean fluorescence intensity (MFI) of cells incubated with functionalized streptavidin minus the MFI of cells incubated without functionalized streptavidin (free fluorophore only) was used to determine binding/uptake.

### Results

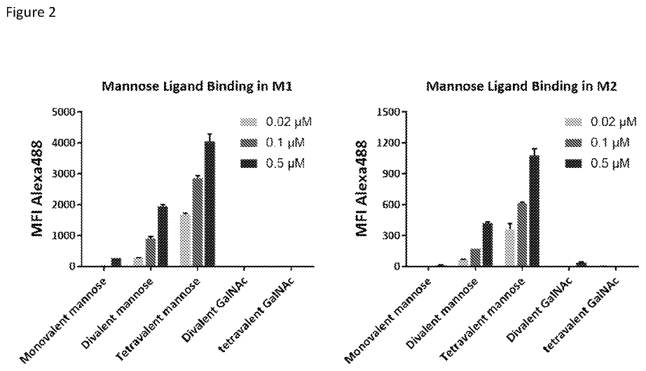

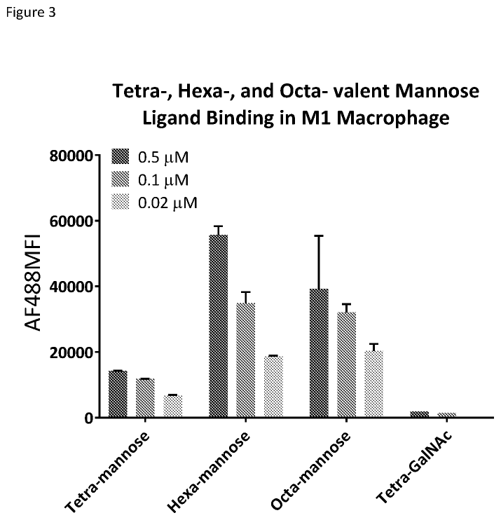

Mono-, di- and tetra-valent mannose ligands were tested in both M1 and M2 macrophages. Dose dependent increase of mannose ligand conjugated fluorescence was observed in both cell types, indicating successful ligand binding. No appreciable binding of divalent or tetravalent GalNAc ligands was observed in either M1 or M2 cells. Consistent with the results that M1 differentiated from human CD14+ monocytes with the current protocol expressed higher level of mannose receptor than M2, all three mannose ligands showed higher binding affinity to M1 than M2 (FIG. 2). In both M1 and M2 cells, the binding activity of mannose ligands correlated positively with valency, with tetravalent ligand showing the highest binding affinity, and monovalent ligand the least. To further optimize the ligand valency, hexa- and octa-valent mannose ligands were synthesized and tested. Hexavalent mannose ligand exhibited further increased binding, while increasing to octa-valency did not show additional benefit (FIG. 3). Therefore, hexavalent mannose conferred the highest binding affinity to M1 macrophages.

### Example 13. Uptake of Fluorescent Labelled Mannose-siRNA Conjugate by M1 Macrophage In Vitro

This example illustrates the uptake of Cy3 fluorescent labelled tetravalent mannose-siRNA conjugate by human CD14+ monocyte derived M1 macrophage in vitro, to demonstrate the receptor involved in uptake. To demonstrate selectivity, two cell types were employed: M1 macrophage, expressing the mannose receptor CD206, and HepG2 cells that express the asialoglycoprotein receptor (ASGPr). Cy3-labeled tetravalent mannose and Cy3-tetravalent GalNAc siRNA conjugates were employed to test the ligand-based siRNA conjugate delivery to these two different cell types.

### Materials and Methods

Differentiation of M1 Macrophages from Human CD14+ Monocytes M1 macrophages were derived from human CD14+ monocytes as described in Example 11.

### HepG2 Cell Culture

HepG2 cells were cultured in MEM medium containing 2 mM L-glutamine, 1 mM sodium pyruvate, 1× non-essential amino acids and 0.15% sodium bicarbonate.

Treatment of Cy3 Labelled Tetravalent Mannose or GalNAc siRNA Conjugates

Human CD14+ monocyte derived M1 macrophages or HepG2 cells were seeded into sterile 4-well chamber slides at 80000 cells/well and 40000 cells/well in OptiMIEM medium, respectively. Cells were cultured at 37° C. after seeding. siRNA conjugate treatment was conduct at 10 minutes post M1 macrophage seeding and 48 h post HepG2 cell seeding. HepG2 cells were washed with 1 mL OptiMIEM before treatment. Each was supplemented with Cy3-tetra-mannose-siCD45 (siRNA 23a) or Cy3-tetra-GalNAc-siCD45 (siRNA 33b) to reach the final concentration of 5 μg/mL in OptiMEM medium. The cells were incubated with the siRNA conjugate for 1, 2 or 4 h. The cells were then washed with PBS 3 times and fixed with 4% PFA for 10 minutes at room temperature. The chambers were removed from the slides and cells mounted with one drop of mounting media containing DAPI staining for the nuclei. The slides were analyzed under fluorescent microscope.

### Results

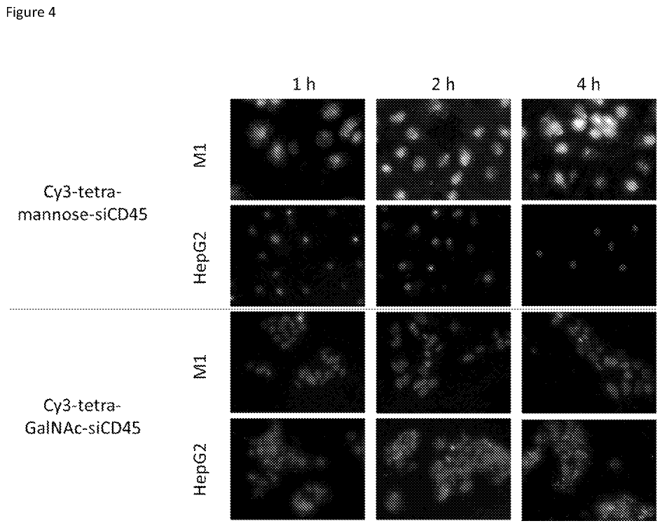

Cy3-tetra-mannose-siRNA treatment resulted in fluorescent detection in M1 macrophages but not in HepG2 cells, while the Cy3-tetra-GalNAc-siRNA showed selective delivery to HepG2 but not M1 macrophage (FIG. 4). The fluorescent signal was detectable at the earliest tested time point (1 hour) and increased at later timepoints (2 hours and 4 hours). These data demonstrate the delivery selectivity of mannose-siRNA conjugates for macrophages, which is expected as they express the appropriate receptor, CD206. Mannose conjugates do not bind to HepG2 cells. Conversely, the GalNAc ligand mediates binding to HepG2 cells (which express ASGP receptor), but not macrophages (which do not express ASGPr).

### Example 14. D-Mannose Competes with Mannose-siRNA Conjugate in M1 Macrophage Binding

This example illustrates competitive inhibition of mannose-siRNA conjugate uptake in M1 macrophages by D-mannose. D-mannose is a natural ligand for the mannose receptor (CD206). To demonstrate the involvement of mannose receptor in the mannose-siRNA conjugate delivery, it was shown that D-mannose competitively inhibits uptake.

### Materials and Methods

Differentiation of M1 Macrophages from Human CD14+ Monocytes

M1 macrophages were derived from human CD14+ monocytes as described in Example 11.

### Treatment of D-Mannose and Cy3-Tetra-Mannose-siCD45

Human M1 macrophages were seeded into sterile 4-well chamber slides at 75000 cells/well in OptiMEM medium. OptiMEM containing D-mannose or D-galactose was added onto testing cells to reach the final concentration of 139 mM and incubated at 37° C. for 5 minutes. Cells were then treated with Cy3-tetra-mannose-siCD45 (siRNA 23a) in OptiMEM with a final concentration of 5 μg/mL and incubated for 1 hour. The cells were then washed with PBS 3 times and fixed with 4% PFA for 10 minutes at room temperature. The chambers were removed from the slides and the cells were mounted with one drop of mounting media containing DAPI staining for the nuclei. The slides were covered, sealed and analyzed under fluorescent microscope.

### Results

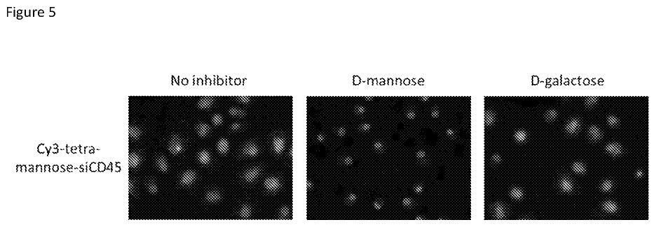

Consistent with Example-9, Cy3-tetra-mannose-siCD45 treatment demonstrated successful delivery to M1 macrophages when treated alone. Competitive binding of D-mannose substantially inhibited the uptake of the mannose-siRNA conjugate as illustrated by the reduced fluorescence (FIG. 5). D-Galactose, which is not a ligand for mannose receptor, showed no competitive binding effect. These results confirmed the involvement of mannose receptor in mannose-siRNA conjugate delivery to human CD14+ monocyte delivery M1 macrophages.

### Example 15. Uptake of Fluorescent Labelled Mannose-siRNA Conjugate by Macrophages and Dendritic Cells In Vitro

This example illustrates the uptake of Cy3 fluorescent labelled hexavalent mannose-siRNA conjugate by human CD14+ monocyte derived M1 and M2 macrophage, immature dendritic cells (iDC) and mature dendritic cells (mDC) in vitro. In addition to M1 and M2 macrophages, mannose receptor CD206 is reportedly expressed in DC, although expression is substantially reduced upon maturation of these cells. Uptake was tested in both iDC and mDC, again characterizing the effect of ligand valency.

### Materials and Methods

Differentiation of M1, M2 Macrophages, iDC and mDC from Human CD14+ Monocytes

M1 macrophages were derived from human CD14+ monocytes as described in Example 11. For iDC and mDC differentiation, isolated human CD14 monocytes cultured in RPMI medium containing 10 mM HEPES, 2 mM L-glutamine and 10% FBS. iDC differentiation was stimulated with treatment of 500 U/mL GM-CSF+1000 U/mL IFNα2b on Day 0 and Day 3. mDC differentiation was triggered with treatment of 500 U/mL GM-CSF+1000 U/mL IFNα2b on Day 0 and 500 U/mL GM-CSF+1000 U/mL IFNα2b+1 μg/mL LPS in combination with 1 μg/mL LPS on Day 3. iDCs and mDCs were collected on Day 5 for conjugate delivery test.

Treatment of Cy3 Labelled Tetravalent Mannose or GalNAc siRNA Conjugates

M1 macrophages, M2 macrophages, iDCs or mDC were seeded into sterile U-bottom 96-well plates at 22500 cells/well in OptiMIEM medium. Cell were treated with Cy3-hexa-mannose-siCD45 (siRNA 32b), Cy3-tetra-mannose-siCD45 (siRNA 23a) or Cy3-tetra-GalNAc-siCD45 (siRNA 33b) at final concentrations of 5, 1.25 or 0.312 μg/mL in OptiMEM medium. At 1 hour post treatment, cells were washed with PBS 3 times and resuspended in stain buffer for flow cytometry analysis.

### Flow Cytometry Analysis

Analyses were performed on a FACS-Canto II using the software FACS Diva. As a marker for viability, cells were stained with Live/Dead green. The forward scatter and side scatter gate were set to include all viable cells. Approximately 10000-15000 cells were counted for each sample and the expression of CD206 was determined as increased intensity in Cy3 detected with the red lase channel. The Cy3 MFI in live cells were calculated.

### Results

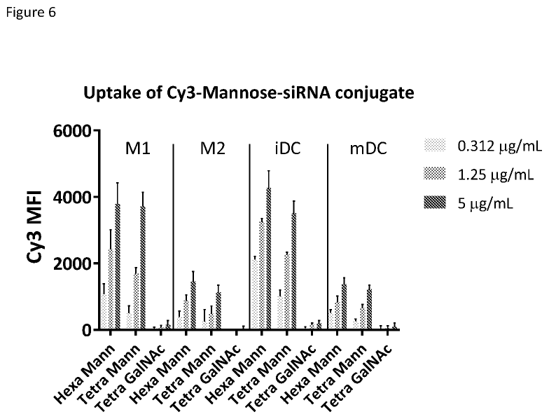

Consistent with Examples 11, 12 and 13, both hexavalent and tetravalent mannose-siRNA conjugates showed superior delivery to M1 than M2 (FIG. 6). Similar delivery selectivity was observed when comparing iDCs with mDCs. Of note, hexa-mannose-siCD45 was more efficient than tetra-mannose-siCD45 in delivering to M1, M2 and iDCs, particularly at the lower dose, which is consistent with the ligand binding data in Example 12. Tetra-GalNAc-siRNA conjugate showed minimal to no delivery to the tested cell types. These results further confirmed that multivalent mannose-conjugates mediate effective uptake to CD206-expressing cells.

### Example 16. Hexavalent Mannose Conjugated siRNA Enabled Gene Silencing in M1 Macrophages In Vitro when Coadministered with Mannose Functionalized Endosome Release Polymer

This example demonstrates in vitro gene silencing mediated by a hexavalent mannose-siRNA conjugate and mannose functionalized endosome release polymer (mannose-ERP) in human CD14+ monocyte derived M1 macrophages. Endosome release is a key bottle neck in siRNA conjugate delivery. To address this challenge, the siRNA treatment was supplemented with a mannose ligand conjugated pH responsive endosome release polymer (Mol Ther. 2021 Oct. 6; 29(10):2910-2919).

### Materials and Methods

Differentiation of M1 Macrophages from Human CD14+ Monocytes

M1 macrophages were derived from human CD14+ monocytes as described in Example 11.

Treatment of siRNA Conjugate and ERP in M1 Macrophages

M1 macrophages were seeded into sterile 96 wells plates (15000/well) and incubated with 20 μg/mL of hexa-mannose-siCD45 (siRNA 32a) or tetra-GalNAc-siCD45 (siRNA 33a) for 4 h at 37° C. Mannose-ERP 40 and GalNAc-ERP 41 were then added to the culture at the concentration of 100 μg/mL. At 24 hours post siRNA treatment, cells were lysed with QuantiGene Lysis Mixture for next step gene expression quantification.

### QuantiGene Branched DNA Assay

To evaluate gene silencing activity, cell lysates collected after the siRNA treatment were subject to QuantiGene branched DNA assay according to manufacturer's protocol. In brief, cell lysates were incubated with capture probes targeting CD45 (target gene) and GAPDH (endogenous control) at 55° C. for 18-20 hours. After washing, the plates were incubated with pre-amplification probes and amplification probes to amplify the signal. The excessive probes were then washed off and assay substrates were added to allow quantifying luminescence using a plate reader. The signal from CD45 was normalized to the signal from GAPDH.

### Results

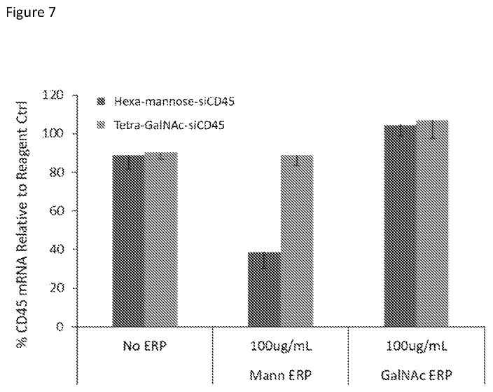

Treatment of hexa-mannose-siCD45 in combination with mannose-ERP but not GalNAc-ERP resulted in target gene inhibition in treated M1 macrophage (FIG. 7). siRNA conjugate treatment alone without ERP was insufficient to induce target gene silencing. RNAi activity was not appreciable in the Tetra-GalNAc-siCD45 treatment groups, regardless of the presence of ERP. These results suggest that mannose ligands support delivery of both siRNA conjugates and ERP to M1 macrophages. The mannose ligand must be present on both entities to ensure their uptake and concomitant gene silencing.

### Example 17. In Vivo Delivery and Activity of Mannose-siRNA Conjugate in Thioglycolate Mouse Model

This example illustrates the in vivo delivery of mannose-siRNA conjugate and gene silencing mediate by mannose-siRNA together with mannose-ERP in peritoneal macrophages stimulated by thioglycolate treatment. Peritoneal macrophages (periMac) elicited by intraperitoneal (IP) injection of thioglycolate in mice are a common source of macrophages for in vitro assays. This Example tests whether the fluorescent labelled mannose-siRNA conjugate could be delivered to thioglycolate induced periMac in vivo and the RNAi activity of mannose-siRNA in the presence of mannose-ERP in this animal model.

### Materials and Methods

### Animal Treatment

C57BL/6 female mice aged 6-8 weeks (n=4 per group) were injected intraperitoneally with 1 mL/animal of 4% thioglycolate in distilled water to elicit macrophages in the peritoneum. Three days post thioglycolate stimulation, animals were injected subcutaneously in the scapular region with a single dose of vehicle control (saline), AF647-hexa-mannose-siCD45 (siRNA 32c, 10 mg/kg), AF647-hexa-mannose-siCD45 (siRNA 32c, 10 mg/kg)+mannose-ERP (50 mg/kg) or mannose-ERP alone (50 mg/kg), using a volume of 5 mL/kg body weight. The peritoneal cells of treated animals were collected at 24 h post siRNA and ERP treatment and analyzed with flow cytometry.

### Flow Cytometry Analysis

Peritoneal cells from each animal were seeded to sterile 96-well V-bottom plates (5E05 cells/well) in triplicates and stained with LIVE/DEAD™ Fixable Violet. Cells were then washed with DPBS and further stained with a phycoerythrin (PE) fluorophore conjugated anti-mouse CD45 antibody and an Allophycocyanin (APC)-efluor780 fluorophore conjugated anti-mouse CD206 antibody. After washing with stain buffer for twice, cells were analyzed with a FACS-Canto II using the software FACS Diva. The expression of CD206, CD45 and delivery of siRNA was assessed with the quantification of fluorescence signals at PE, APC-cy7 and AF647, respectively. Silencing of the target gene (CD45) was determined by normalizing CD45 expression in the treated animals to the control animals.

### Results

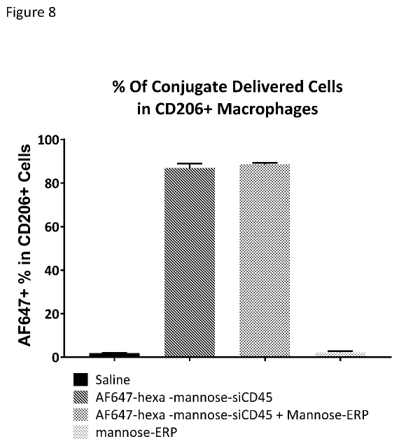

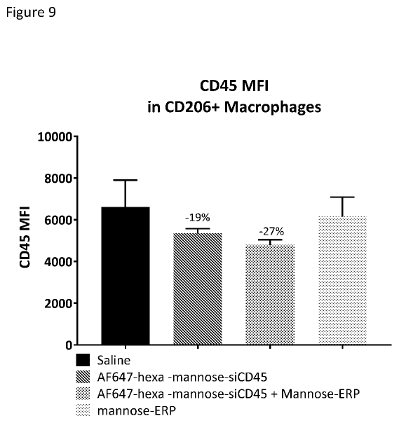

siRNA delivery was detected in >80% CD206+ peritoneal macrophages (periMacs) in AF647-hexa-mannose-siCD45 treated animals, regardless of the presence/absence of mannose-ERP (FIG. 8). The delivered siCD45 enabled a trend of target gene silencing in both groups, with mannose-ERP showing a slight benefit to RNAi activity (FIG. 9), but neither treatment achieved statistically significant gene knockdown (one-way ANOVA analysis). The expression of CD206 in these thioglycolate elicited periMAcs may not have been high enough to allow sufficient siRNA conjugate uptake. Nonetheless, these results demonstrate successful delivery of hexa-mannose conjugated siRNA to macrophages in the thioglycolate mouse model.

### Example 18. Combination Treatment of Hexa-Mannose-siRNA and GalNAc-siRNA Demonstrated Anti-Viral Potency in a Guinea Pig Viral Challenge Model

This example illustrates the in vivo antiviral efficacy of hexa-mannose-siRNA and GalNAc-siRNA combination treatment in a guinea pig viral challenge model. Marburg virus (MARV) is a hemorrhagic fever virus of the Filoviridae family of viruses. Challenging guinea pigs with a lethal dose of MARV results in ˜100% mortality rate if no anti-viral treatment is provided. MARV infection affects multiple liver cell types including hepatocytes, macrophages and dendritic cells. Hepatocytes express ASGPR, enabling uptake of GalNAc-siRNAs. Macrophages and dendritic cells express CD206, that enable uptake of mannose-siRNA conjugates as evidenced by previous examples in this patent. To create a therapeutic product for MARV that would successfully address the virus in all relevant cell types, siRNA targeting MARV transcripts were designed and conjugated to both GalNAc and mannose ligands. These conjugates were tested alone and in combination in the guinea pig MARV infection model.

### Materials and Methods

### Animal Treatment

Guinea pigs (n=5 per group) were injected intraperitoneally with 1000 pfu/animal of MARV Angola strain. Twenty-four-hour post viral exposure, animals were injected subcutaneously in the scapular region with tetra-GalNAc-siMARV (siRNA 33c) alone or in combination with hexa-mannose-siMARV (siRNA 32d) (1:1) (5 mg/kg total siRNA, daily SQ dosing×7 doses or weekly SQ dose×4 doses). One group with a single 10 mg/kg total siRNA dose of GalNAc and mannose conjugates combination was also tested. The antiviral efficacy was evaluated by bodyweights, survival rates and viremia.

### Results

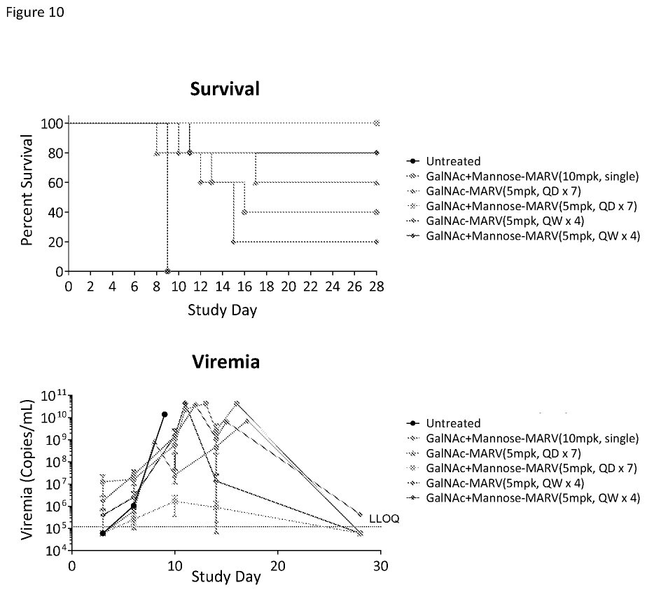

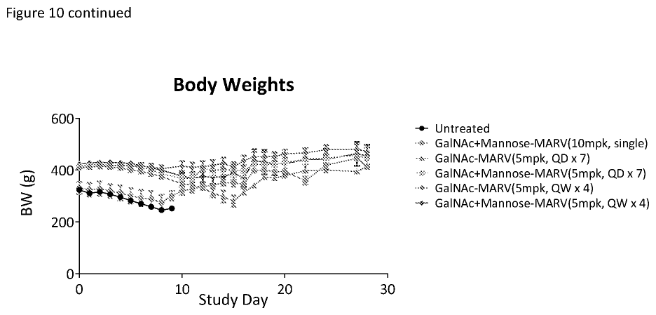

In both daily and weekly dosing groups, the GalNAc/mannose conjugates combination treatment again provided superior protection as compared to the GalNAc conjugate alone groups (FIG. 10). In particular, the daily dosing group with 5 mg/kg of total GalNAc/mannose conjugate achieved 100% survival. The viremia and body weight data agreed well with the animal survival results. Together, the mannose/GalNAc combination ligand platform appears to offer superior protection in the MARV guinea pig model.

### Example 19. Hexavalent Mannose Conjugated siRNA with or without Mannose Functionalized Endosome Release Polymer Enabled Gene Silencing in Liver and Spleen Macrophages In Vivo

This example demonstrates in vivo gene silencing mediated by a hexavalent mannose-siRNA conjugate and mannose functionalized endosome release polymer (mannose-ERP) in mouse liver and spleen macrophage. Data from Example 12 demonstrated that mannose functionalized endosome release polymer (mannose-ERP) could improve the RNAi activity of mannose-siRNA conjugate in human macrophages. This strategy is tested here in mice.

### Materials and Methods

### Animal Treatment

C57BL/6 female mice aged 6-8 weeks (n=4 per group) were injected subcutaneously with a single dose of 3 mg/kg hexa-mannose conjugated siRNA targeting mouse Hprt1 gene (hexa-mannose-siHprt1, siRNA 32e) or unconjugated siHprt1, either alone or together with mannose-ERP at 20 mg/kg. At Day 7 post treatment, the liver and spleen were collected from treated animals. The liver tissues were dissociated with mouse liver dissociation kit using a gentleMACS™ Octo Dissociator (Miltenyi Biotec) and filtered through a 0.2 μm cell strainer. The spleen tissues were minced by the flat end of the 5 mL syringe plunger and filtered through a 0.2 μm cell strainer. Macrophages were isolated from the prepared single liver/spleen cell suspension using Anti-mouse F4/80 MicroBeads (Miltenyi Biotec) and lysed with QuantiGene Assay lysis mixture.

### QuantiGene Branched DNA Assay

To evaluate gene silencing activity, cell lysates from the collected liver and spleen macrophages were subject to QuantiGene branched DNA assay according to manufacturer's protocol. In brief, cell lysates were incubated with capture probes targeting mHprt1 (target gene) and mGapdh (endogenous control) at 55° C. for 18-20 h. After washing, the plates were incubated with pre-amplification probes and amplification probes to amplify the signal. The excessive probes were then washed off and assay substrates were added to allow quantifying luminescence using a plate reader. The signal from mHprt1 was normalized to the signal from mGapdh.

### Results

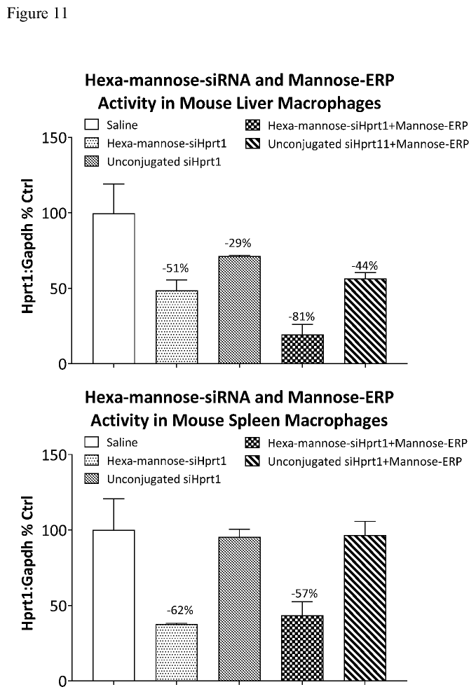

Treatment of hexa-mannose-siHprt1 alone at a single dose of 3 mg/kg resulted in 51% and 62% target gene silencing in liver and spleen macrophages, respectively (FIG. 11). The treatment of unconjugated siHprt1 showed minimal to none RNAi effect. Co-administrated of mannose-ERP substantially improved the activity of hexa-mannose-siHprt1 in liver macrophages but showed no appreciable effect in the spleen macrophages. The results demonstrated the in vivo activity of hexa-mannose conjugated siRNA and the benefit of mannose-ERP co-treatment in liver macrophages.

### Example 20. Dose Response of Mannose-ERP in Liver Macrophages In Vivo

This example demonstrates the dose response effect of mannose functionalized endosome release polymer (mannose-ERP) in mouse liver macrophages in vivo.

### Materials and Methods

### Animal Treatment

C57BL/6 female mice aged 6-8 weeks (n=4 per group) were injected subcutaneously with a single dose of 3 mg/kg hexa-mannose-siHprt1 (siRNA 32e), either alone or together with mannose-ERP at 5, 10 or 20 mg/kg. At Day 7 post treatment, the liver and spleen were collected from treated animals. The liver tissues were dissociated with mouse liver dissociation kit using a gentleMACS™ Octo Dissociator (Miltenyi Biotec) and filtered through a 0.2 μm cell strainer. Macrophages were isolated from the prepared single liver cell suspension using Anti-mouse F4/80 MicroBeads (Miltenyi Biotec) and lysed with QuantiGene Assay lysis mixture.

### QuantiGene Branched DNA Assay

To evaluate gene silencing activity, cell lysates from the collected liver macrophages were subject to QuantiGene branched DNA assay according to manufacturer's protocol. In brief, cell lysates were incubated with capture probes targeting mHprt1 (target gene) and mGapdh (endogenous control) at 55° C. for 18-20 h. After washing, the plates were incubated with pre-amplification probes and amplification probes to amplify the signal. The excessive probes were then washed off and assay substrates were added to allow quantifying luminescence using a plate reader. The signal from mHprt1 was normalized to the signal from mGapdh.

### Results

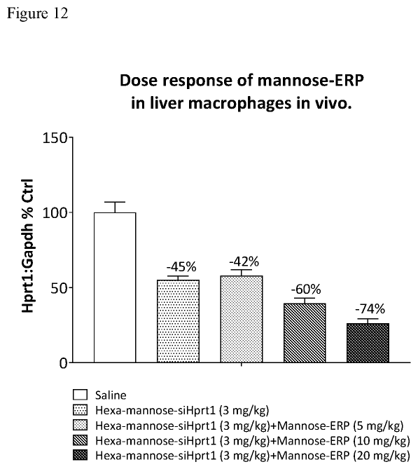

Treatment of hexa-mannose-siHprt1 alone at a single dose of 3 mg/kg resulted in 45% target gene silencing in liver macrophages (FIG. 12). Dose response effect observed with the co-treated mannose-ERP. 5 mg/kg mannose-ERP showed no appreciable effect on RNAi activity. 10 and 20 mg/kg of mannose-ERP improved the activity of hexa-mannose-siRNA in a dose responsible manner.

### Example 21. Dose Response of Hexa-Mannose-siRNA in Liver and Spleen Macrophages In Vivo

This example demonstrates the dose response effect of hexa-mannose-siRNA in mouse liver and spleen macrophages in vivo.

### Materials and Methods

### Animal Treatment

C57BL/6 female mice aged 6-8 weeks (n=4 per group) were injected subcutaneously with a single dose of 1, 3 or 9 mg/kg hexa-mannose-siHprt1 (siRNA 32e) together with mannose-ERP at 20 mg/kg. At Day 7 post treatment, the liver and spleen were collected from treated animals. The liver tissues were dissociated with mouse liver dissociation kit using a gentleMACS™ Octo Dissociator (Miltenyi Biotec) and filtered through a 0.2 μm cell strainer. The spleen tissues were minced by the flat end of the 5 mL syringe plunger and filtered through a 0.2 μm cell strainer. Macrophages were isolated from the prepared single liver/spleen cell suspension using Anti-mouse F4/80 MicroBeads (Miltenyi Biotec) and lysed with QuantiGene Assay lysis mixture.

### QuantiGene Branched DNA Assay

To evaluate gene silencing activity, cell lysates from the collected liver and spleen macrophages were subject to QuantiGene branched DNA assay according to manufacturer's protocol. In brief, cell lysates were incubated with capture probes targeting mHprt1 (target gene) and mGapdh (endogenous control) at 55° C. for 18-20 h. After washing, the plates were incubated with pre-amplification probes and amplification probes to amplify the signal. The excessive probes were then washed off and assay substrates were added to allow quantifying luminescence using a plate reader. The signal from mHprt1 was normalized to the signal from mGapdh.

### Results

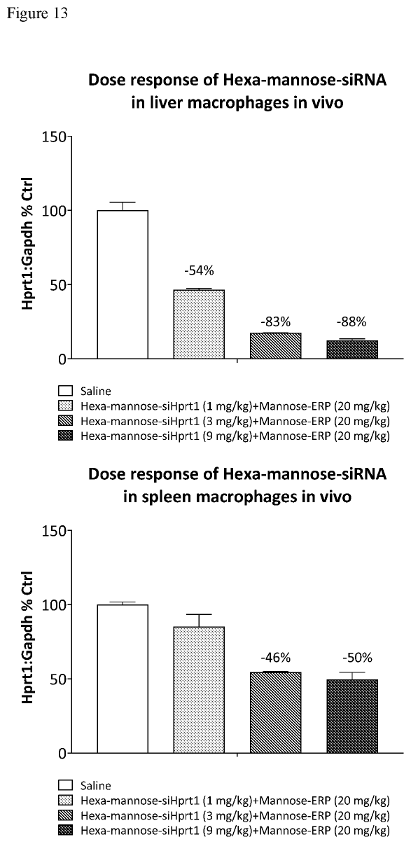

Dose responsive RNAi activity of hexa-mannose-siRNA was observed in both liver and spleen macrophages (FIG. 13). Single dose of 1 mg/kg hexa-mannose-siHprt1 with 20 mg/kg of mannose-ERP resulted in 54% reduction of target gene in liver macrophages while showed no appreciable knockdown effect in spleen macrophages. Increasing the dose of the conjugate to 3 and 9 mg/kg substantially enhanced the gene silencing effect in both liver and spleen macrophages.

### Example 22. Duration of Effect of Hexa-Mannose-siRNA with Mannose-ERP in Liver and Spleen Macrophages In Vivo

This example demonstrates the duration of effect of hexa-mannose-siRNA with mannose-ERP in mouse liver and spleen macrophages in vivo.

### Materials and Methods

### Animal Treatment

C57BL/6 female mice aged 6-8 weeks (n=4 per group) were injected subcutaneously with a single dose of 3 mg/kg hexa-mannose-siHprt1 (siRNA 32e) together with mannose-ERP at 20 mg/kg. At Day 2, 7, 14, 21, 28 and 35 post treatment, the liver and spleen were collected from treated animals. The liver tissues were dissociated with mouse liver dissociation kit using a gentleMACS™ Octo Dissociator (Miltenyi Biotec) and filtered through a 0.2 μm cell strainer. The spleen tissues were minced by the flat end of the 5 mL syringe plunger and filtered through a 0.2 μm cell strainer. Macrophages were isolated from the prepared single liver/spleen cell suspension using Anti-mouse F4/80 MicroBeads (Miltenyi Biotec) and lysed with QuantiGene Assay lysis mixture.

### QuantiGene Branched DNA Assay

To evaluate gene silencing activity, cell lysates from the collected liver and spleen macrophages were subject to QuantiGene branched DNA assay according to manufacturer's protocol. In brief, cell lysates were incubated with capture probes targeting mHprt1 (target gene) and mGapdh (endogenous control) at 55° C. for 18-20 h. After washing, the plates were incubated with pre-amplification probes and amplification probes to amplify the signal. The excessive probes were then washed off and assay substrates were added to allow quantifying luminescence using a plate reader. The signal from mHprt1 was normalized to the signal from mGapdh.

### Results

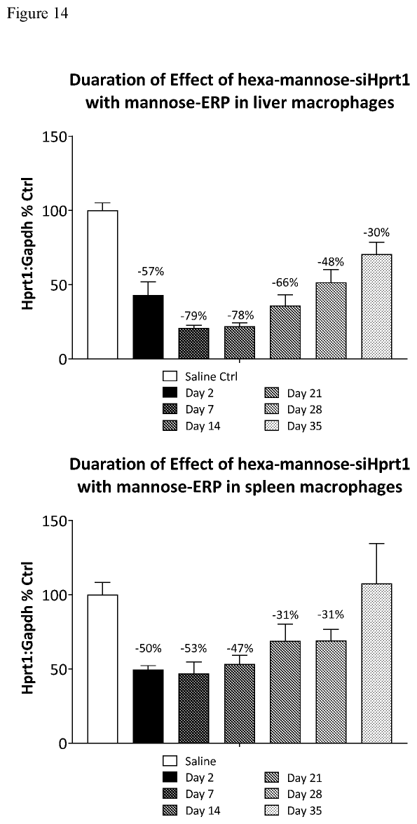

At Day 2 post treatment, single SQ dose of 3 mg/kg hexa-mannose-mHPRT1 with 20 mg/kg mannose-ERP resulted in 57% and 50% target silencing in liver and spleen macrophages, respectively (FIG. 14). In liver macrophages, more marked reduction (˜80%) of target gene was observed at Day 7 and 14 with gradual rebound of the expression from Day 21 post treatment. In spleen macrophage, the ˜50% gene silencing maintain for 2 weeks and start to rebound from Day 21. The RNAi activity diminished at around Day 35 post treatment. These results suggest that the gene silencing effect of the hexa-mannose-siRNA with mannose-ERP is durable for around 5 weeks.

### Example 23. Multi-Dosing of Mannose Conjugate with ERP Improves Target Silencing In Vivo

This example demonstrates the duration of effect of hexa-mannose-siRNA with mannose-ERP in mouse liver and spleen macrophages in vivo.

### Materials and Methods

### Animal Treatment

C57BL/6 female mice aged 6-8 weeks (n=4 per group) were injected subcutaneously with 3 mg/kg hexa-mannose-siHprt1 (siRNA 32e) together with mannose-ERP at 20 mg/kg at different dosing regimens (single dose, weekly dose×5 or bi-weekly dose×3). At Day 7 post the final dose of treatment, the liver and spleen were collected from treated animals. The liver tissues were dissociated with mouse liver dissociation kit using a gentleMACS™ Octo Dissociator (Miltenyi Biotec) and filtered through a 0.2 μm cell strainer. The spleen tissues were minced by the flat end of the 5 mL syringe plunger and filtered through a 0.2 μm cell strainer. Macrophages were isolated from the prepared single liver/spleen cell suspension using Anti-mouse F4/80 MicroBeads (Miltenyi Biotec) and lysed with QuantiGene Assay lysis mixture.

### QuantiGene Branched DNA Assay

To evaluate gene silencing activity, cell lysates from the collected liver and spleen macrophages were subject to QuantiGene branched DNA assay according to manufacturer's protocol. In brief, cell lysates were incubated with capture probes targeting mHprt1 (target gene) and mGapdh (endogenous control) at 55° C. for 18-20 h. After washing, the plates were incubated with pre-amplification probes and amplification probes to amplify the signal. The excessive probes were then washed off and assay substrates were added to allow quantifying luminescence using a plate reader. The signal from mHprt1 was normalized to the signal from mGapdh.

### Results

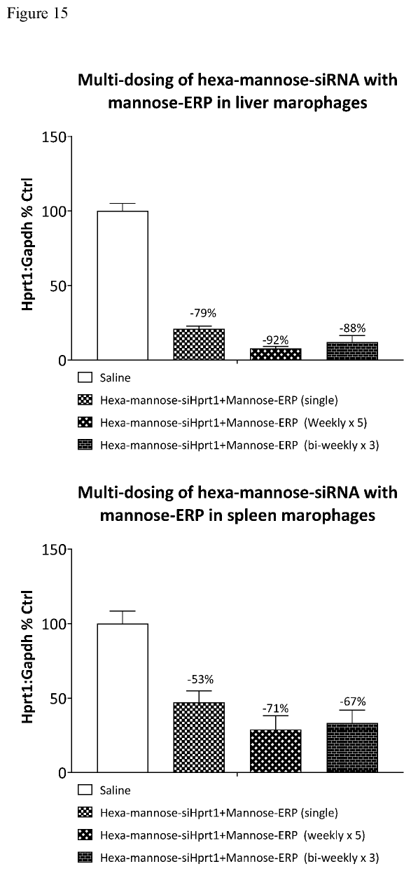

At Day 7 post treatment, single SQ dose of 3 mg/kg hexa-mannose-mHPRT1 with 20 mg/kg mannose-ERP resulted in 79% and 53% target silencing in liver and spleen macrophages, respectively (FIG. 15). Multi-dosing of mannose-siRNA conjugate and mannose-ERP resulted in substantial improvement in target gene silencing. Weekly dosing of 3 mg/kg hexa-mannose-mHPRT1 with 20 mg/kg mannose-ERP for 5 weeks achieved 92% and 71% target gene knockdown in liver and spleen macrophages, respectively. Biweekly dosing for 3 weekly enabled slightly lower silencing effect.

What is claimed is:

1. A compound of formula I 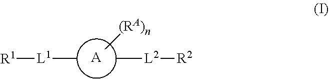
wherein:
R¹ a is targeting ligand that comprises one or more groups of formula: 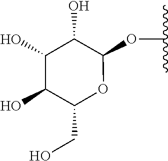 [SMILES: *O[C@H]1O[C@H](CO)[C@@H](O)[C@H](O)[C@@H]1O]
L¹ is absent or a linking group;
L² is absent or a linking group;
R² is an oligonucleotide or a group of formula: 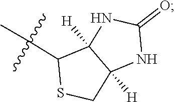 [SMILES: *C1SC[C@@H]2NC(=O)N[C@H]12]
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydroxy, CN, F, Cl, Br, I, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy; and
n is 0, 1, 2, 3, 4, 5, 6, 7, 8, 9, or 10;
or a salt thereof.

2. The compound or salt of claim 1, wherein R¹ is selected from the group consisting of: 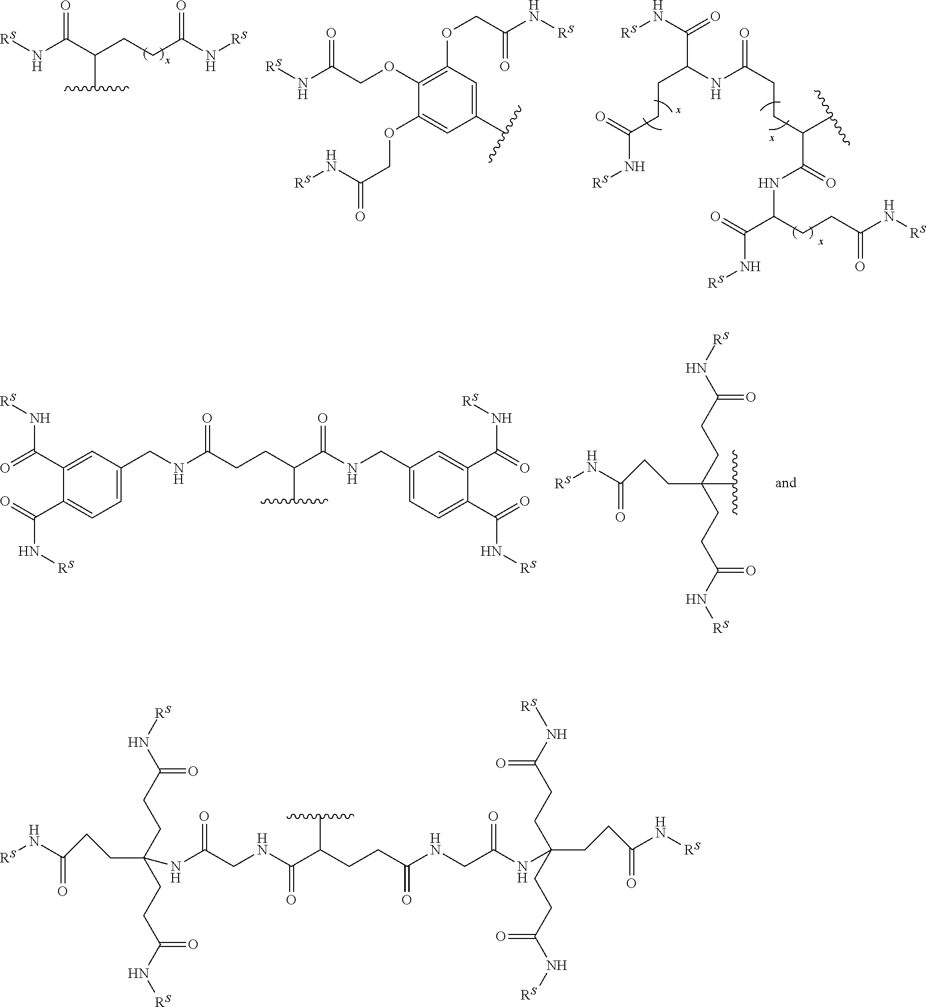
wherein
R^{S} is 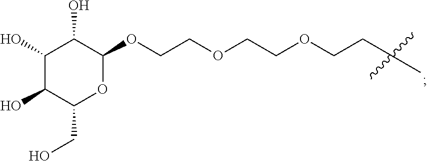 [SMILES: *CCOCCOCCO[C@H]1O[C@H](CO)[C@@H](O)[C@H](O)[C@@H]1O]
n is 2, 3, or 4; and
x is 1 or 2.

3. The compound or salt of claim 1, wherein R¹ is: 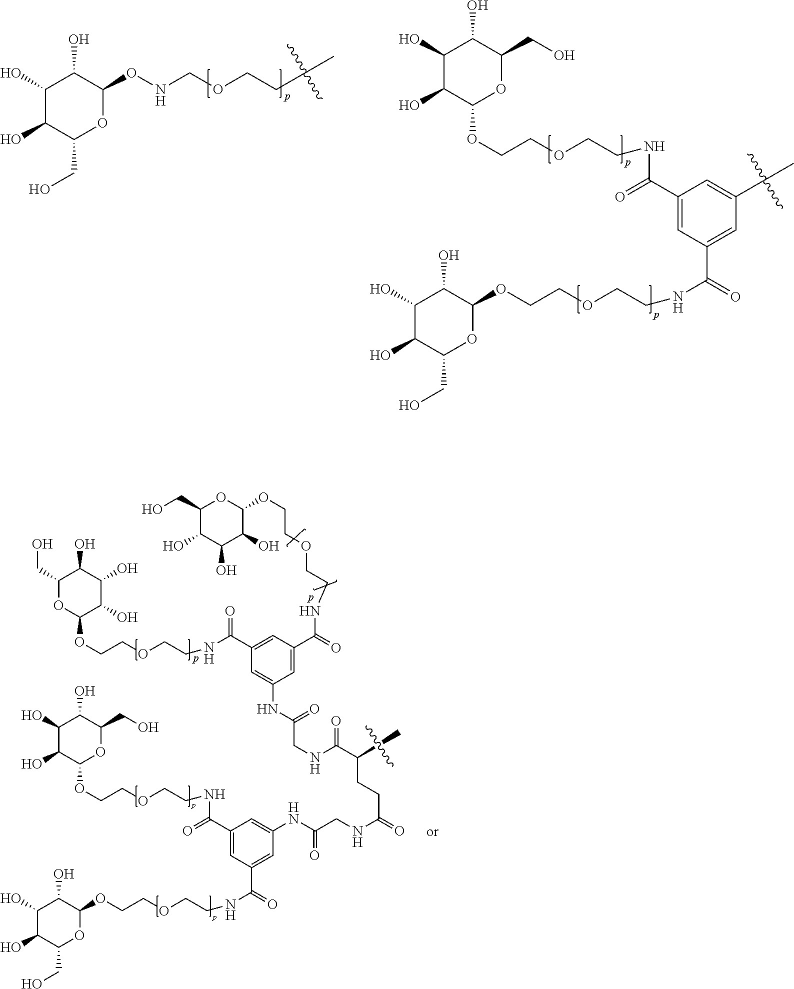 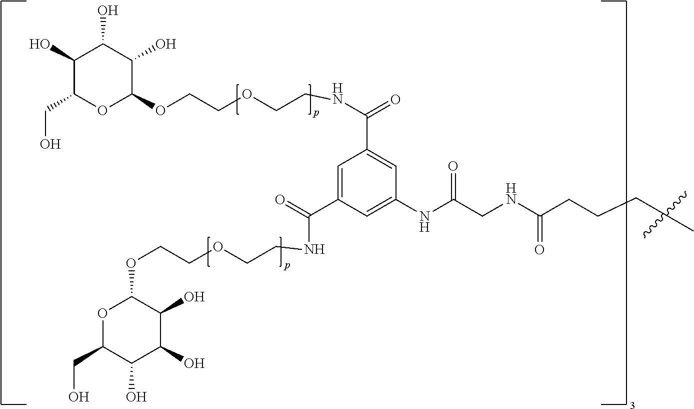
or a salt thereof wherein each p is independently selected from the group consisting of 0, 1, 2, 3, 4, or 5.

4. The compound or salt of claim 1, wherein R¹ is: 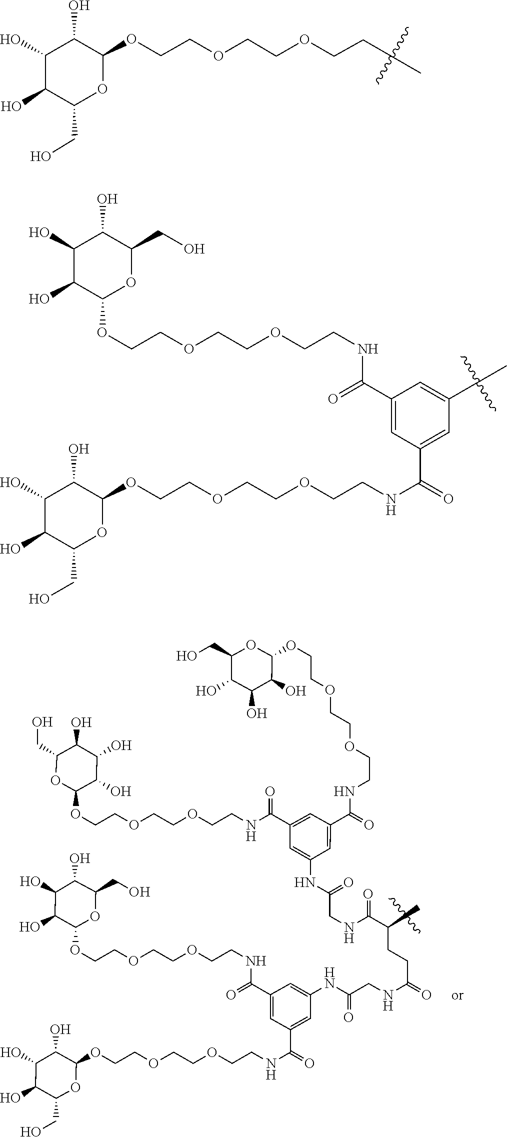 [SMILES: *CCOCCOCCO[C@H]1O[C@H](CO)[C@@H](O)[C@H](O)[C@@H]1O ; *c1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1 ; C[C@@H](CCC(=O)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1)C(=O)NCC(=O)Nc1cc(C(=O)NCCOCCOCCO[C@H]2O[C@H](CO)[C@@H](O)[C@H](O)[C@@H]2O)cc(C(=O)NCCOCCOCCO[C@H]2O[C@H](CO)[C@@H](O)[C@H](O)[C@@H]2O)c1] 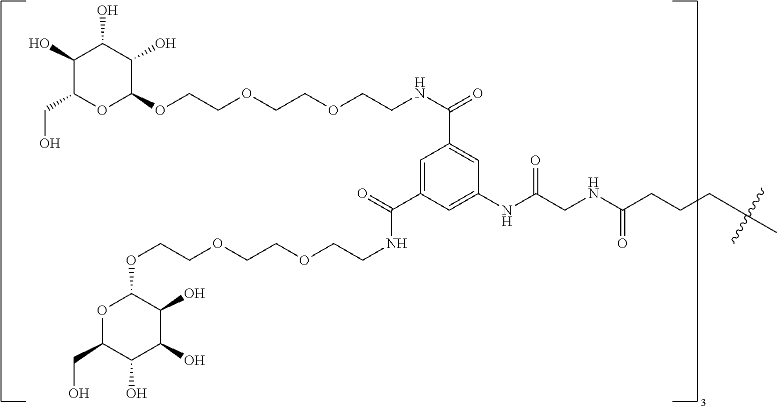
or a salt thereof.

5. The compound or salt of any one of claims 1-4, wherein Li is a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

6. The compound or salt of any one of claims 1-4, wherein L¹ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

7. The compound or salt of any one of claims 1-4, wherein L¹ is a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

8. The compound or salt of any one of claims 1-4, wherein L is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

9. The compound or salt of any one of claims 1-4, wherein L¹ is selected from the group consisting of:

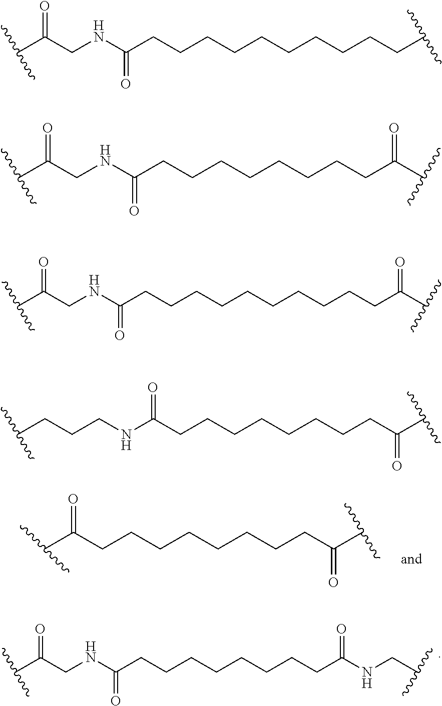 [SMILES: *CCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O ; *CNC(=O)CCCCCCCCC(=O)NCC(*)=O]

10. The compound or salt of any one of claims 1-4, wherein L¹ is selected from the group consisting of:

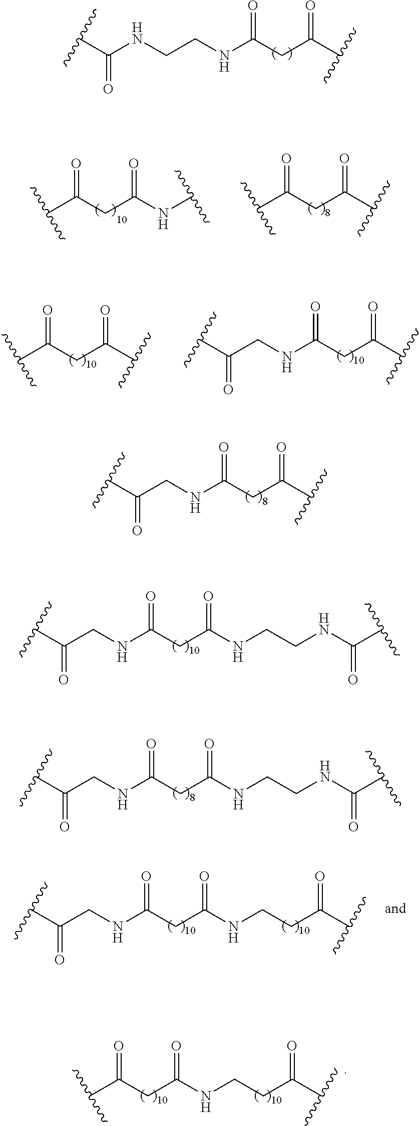

11. The compound or salt of any one of claims 1-4, wherein L¹ is selected from the group consisting of:

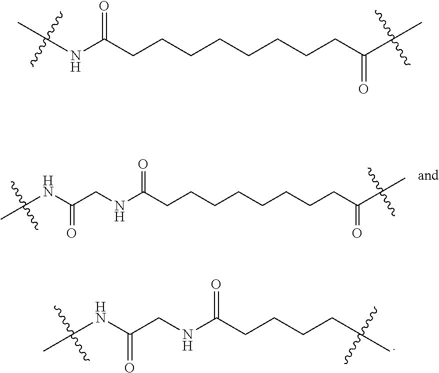 [SMILES: *NC(=O)CCCCCCCCC(*)=O ; *NC(=O)CNC(=O)CCCCCCCCC(*)=O ; *CCCCC(=O)NCC(=O)N*]

12. The compound or salt of any one of claims 1-11, wherein L² is connected to R² through —O—.

13. The compound or salt of any one of claims 1-11, wherein L² is —CH₂—, —CH₂CH₂—, or —CH(OH)CH₂—.

14. The compound or salt any one of claims 1-11, wherein L² is C₁₋₄ alkylene- that is optionally substituted with hydroxy.

15. The compound or salt of any one of claims 1-14, wherein A is absent, phenyl, pyrrolidinyl, or cyclopentyl.

16. The compound or salt of any one of claims 1-14 that is a compound formula (Ic): 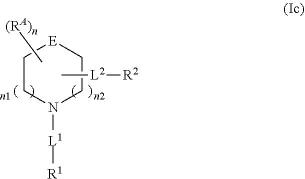
or a salt thereof, wherein:
E is —O— or —CH₂—;
n is selected from the group consisting of 0, 1, 2, 3, and 4; and
n1 and n2 are each independently selected from the group consisting of 0, 1, 2, and 3;
or a salt thereof.

17. The compound or salt of any one of claims 1-14 that is a compound formula (Ig): 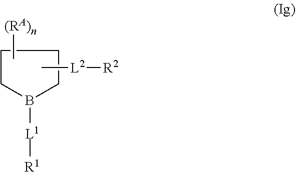
or a salt thereof, wherein:
B is —N— or —CH—;
L² is C₁₋₄ alkylene- that is optionally substituted with hydroxyl or halo; and
n is 0, 1, 2, 3, 4, 5, 6, or 7;
or a salt thereof.

18. The compound or salt of any one of claims 1-17, wherein each R^{A} is independently hydroxy or C₁₋₈ alkyl that is optionally substituted with hydroxyl.

19. The compound or salt of any one of claims 1-17, wherein each R^{A} is independently selected from the group consisting of hydroxy, methyl and —CH₂OH.

20. The compound or salt of any one of claims 1-11 that is selected from the group consisting of: 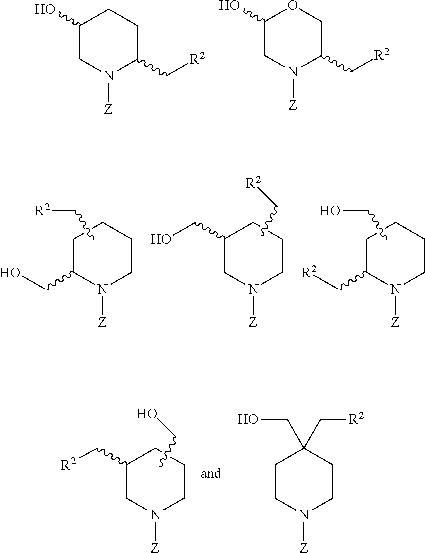
wherein: Z is -L¹-R¹; and salts thereof.

21. The compound or salt of any one of claims 1-11, wherein the -A-L²-R² moiety is: 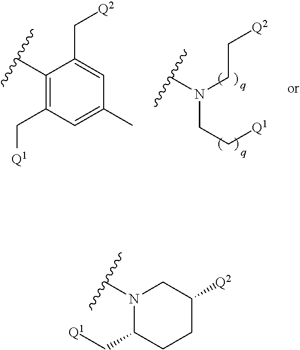
wherein:
Q¹ is hydrogen and Q² is R²; or Q¹ is R² and Q² is hydrogen; and
each q is independently 0, 1, 2, 3, 4 or 5.

22. The compound or salt of any one of claims 1-11 that is selected from the group consisting of: 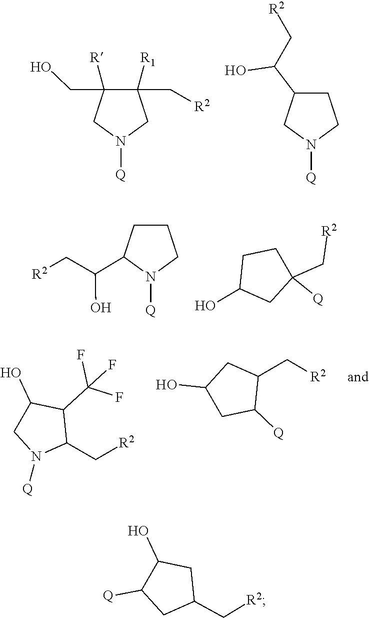
wherein Q is -L¹-R¹; and
R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
and salts thereof.

23. The compound or salt of claim 1 that is selected from the group consisting of:

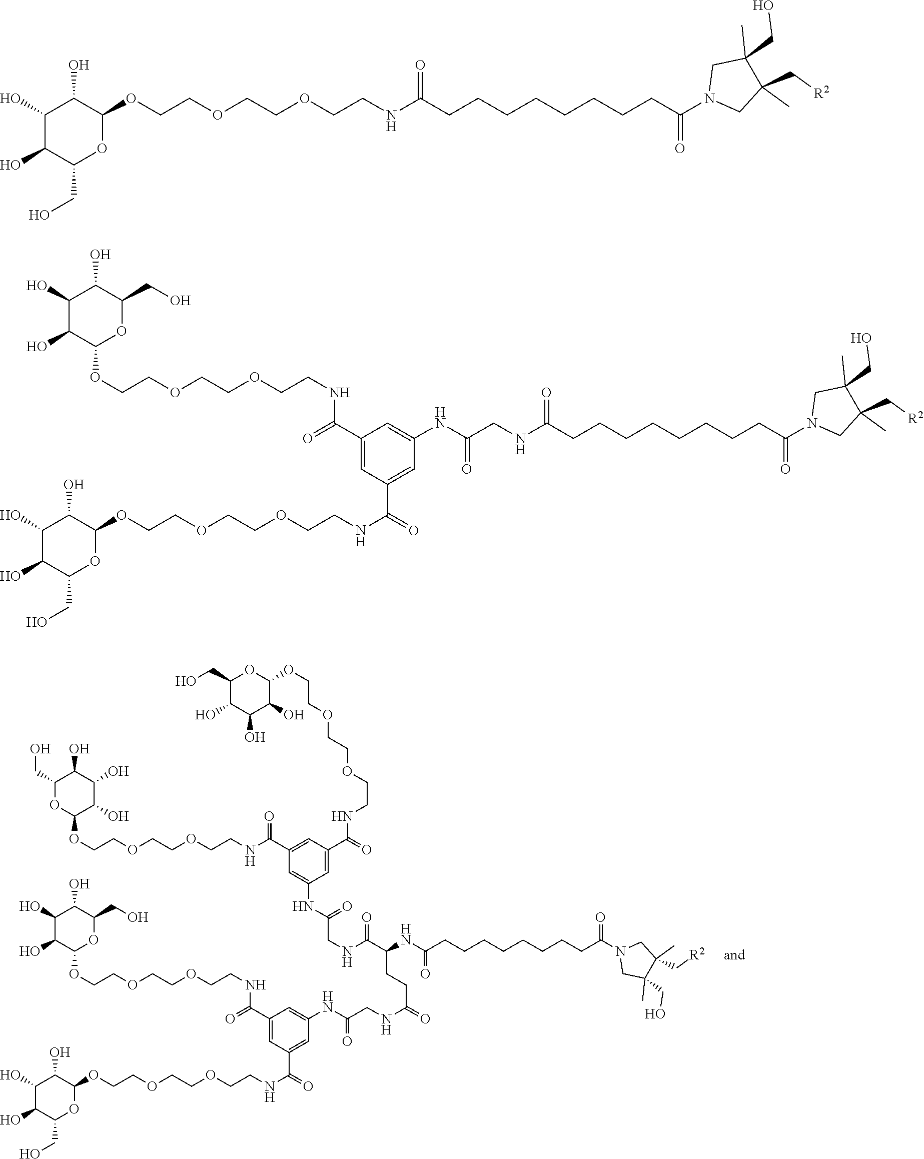

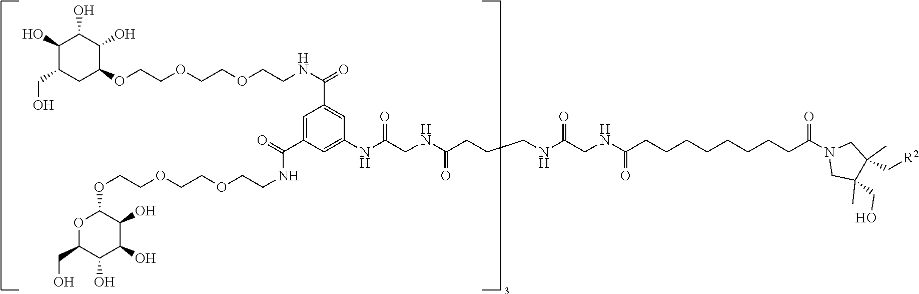

24. The compound or salt of claim 1 that is selected from the group consisting of: 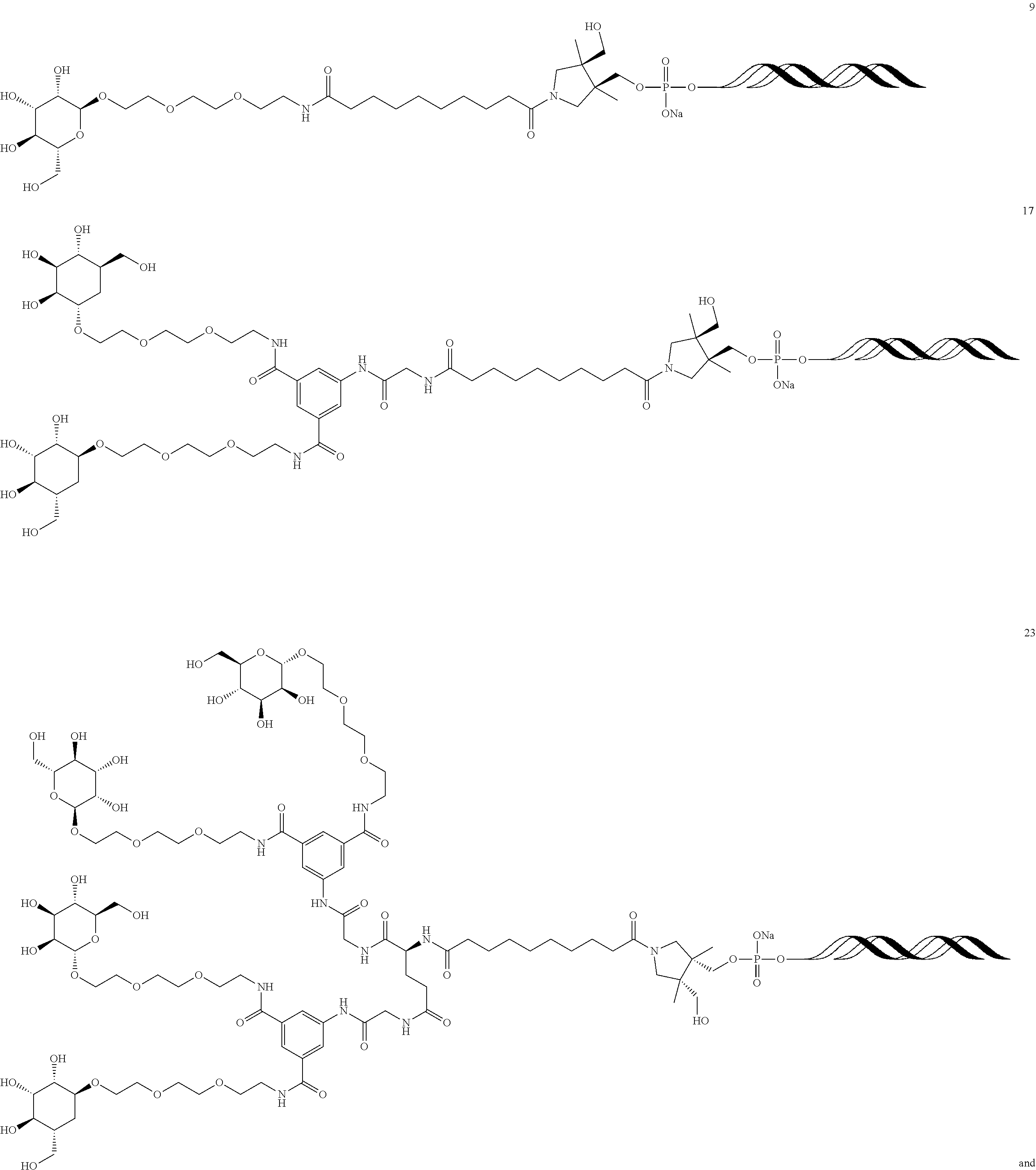 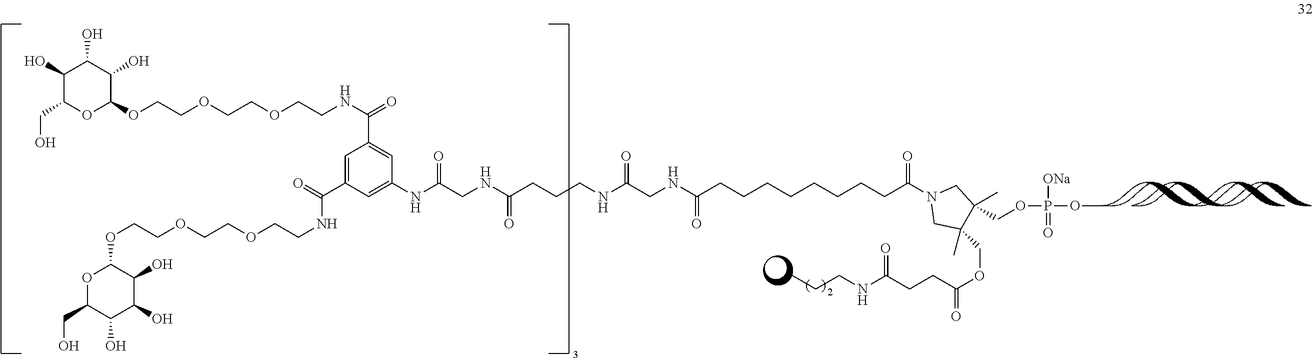
and salts thereof, wherein the symbol: [symbol]
represents an oligonucleotide.

25. The compound or salt of claim 24, wherein the oligonucleotide is an siRNA.

26. The compound or salt of claim 25, wherein the siRNA is attached to the reminder of the compound through an oxygen of a phosphate at the 3′-end of the sense strand of the siRNA.

27. A pharmaceutical composition comprising a compound as described in any one of claims 1-26 or a pharmaceutically acceptable salt thereof, and a pharmaceutically acceptable carrier.

28. A method to deliver an oligonucleotide to an animal comprising administering a compound of formula I, as described in any one of claims 1-26 or a pharmaceutically acceptable salt thereof, to the animal.

29. A method to deliver an oligonucleotide to a cell, comprising contacting the cell with a compound of formula I, as described in any one of claims 1-26 or a pharmaceutically acceptable salt thereof, wherein the cell is in vitro or in vivo.

30. A method to deliver an oligonucleotide to cells that express mannose receptor CD206 in an animal comprising administering a compound of formula I, as described in any one of claims 1-26 or a pharmaceutically acceptable salt thereof, to the animal.

31. The method of claim 30, wherein the cells that express mannose receptor CD206 in the animal are macrophages or dendritic cells.

32. A method to treat a disease selected from the group consisting of acute liver disease, liver inflammation, rheumatoid arthritis, and a flavoviral infection in an animal comprising administering a compound of formula I, as described in any one of claims 1-26 or a pharmaceutically acceptable salt thereof, to the animal.

33. A compound of formula I as described in any one of claims 1-26 or a pharmaceutically acceptable salt thereof for delivering an oligonucleotide to an animal.

34. A compound of formula I as described in any one of claims 1-26 or a pharmaceutically acceptable salt thereof for delivering an oligonucleotide to cells that express mannose receptors CD206.

35. The compound or pharmaceutically acceptable salt of claim 34, wherein the cells that express mannose receptors CD206 in the animal are macrophages or dendritic cells.

36. A compound of formula I as described in any one of claims 1-26 or a pharmaceutically acceptable salt thereof for the prophylactic or therapeutic treatment of a disease selected from the group consisting of acute liver disease, liver inflammation, rheumatoid arthritis, and a flavoviral infection.

37. The use of a compound of formula I as described in any one of claims 1-26 or a pharmaceutically acceptable salt thereof to prepare a medicament for delivering an oligonucleotide to an animal.

38. The use of a compound of formula I as described in any one of claims 1-26 or a pharmaceutically acceptable salt thereof to prepare a medicament for delivering an oligonucleotide to cells that express mannose receptors CD206.

39. The use of claim 38, wherein the cells that express mannose receptors CD206 in the animal are macrophages or dendritic cells.

40. The use of a compound of formula I as described in any one of claims 1-26 or a pharmaceutically acceptable salt thereof to prepare a medicament for treating a disease selected from the group consisting of acute liver disease, liver inflammation, rheumatoid arthritis, and a flavoviral infection.

41. The method or use of any one of claims 28-40, wherein the administration or use of the compound of formula I is in combination with a compound of the following formula XX:
T⁵-L-[PEGMAₘ-M²ₙ]ᵥ-[DMAEMA_{q}-PAAᵣ-BMAₛ]_{w} (XX)
wherein:
PEGMA is polyethyleneglycol methacrylate residue with 2-20 ethylene glycol units;
M² is a methacrylate residue selected from the group consisting of
a (C₄-C₁₈)alkyl-methacrylate residue;
a (C₄-C₁₈)branched alkyl-methacrylate residue;
a cholesteryl methacrylate residue;
a (C₄-C₁₈)alkyl-methacrylate residue substituted with one or more fluorine atoms; and
a (C₄-C₁₈)branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa;
w is 1 to 25 kDa;
T⁵ is a targeting moiety; and
L is absent or is a linking moiety.

42. The method or use of claim 41, wherein the method or use comprises delivery to macrophages in the liver.

43. The method of use of claim 41, wherein the compound of formula I and compound of formula XX are administered separately.

44. The method of use of claim 41, wherein the compound of formula XX is administered after administration of the compound of formula I.

45. The method of use of claim 41, wherein the compound of formula I and compound of formula XX are administered together within a single composition.

46. The method or use of any one of claims 41-45, wherein the compound of formula XX is Mannose ERP 40

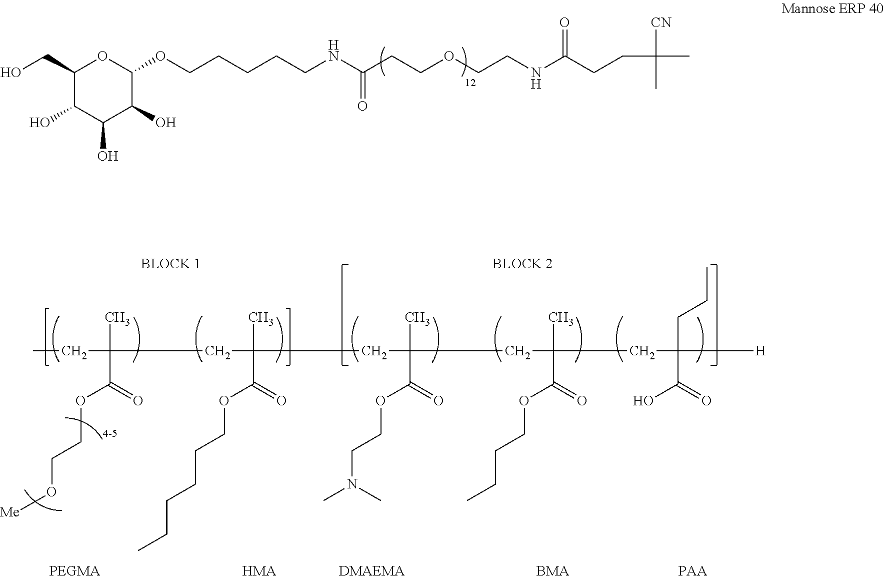

47. A compostion comprising a compound of formula I as described in any one of claims 1-26 or a pharmaceutically acceptable salt thereof and a compound of formula XX as described in claim 41 or claim 46.

48. A combination that comprises:
a) a compound of formula I as described in any one of claims 1-26 or a pharmaceutically acceptable salt thereof; and
b) a compound of formula XX as described in claim 41 or claim 46 or a pharmaceutically acceptable salt thereof.
